SEQUENCE LISTING (413 sequences)

SEQ ID NO: 1 - DNA, 28690 bp, Homo sapiens
tgtgagctactgtgtctggcctgaataataaaatttaaaacaatttttcaaaaattcaccatgaggtctcactatattccctaggctggtctcaaacccctggactccaagtgatccaccccaccttcccgagtagctgggactagagatgcacaccattgcacccaatagagcaatacgtttctgttctttgtaaattacctgctctaaggtatttttgttatagcagcctatatggactaagctgacttgtaacgttacttgagactttaaagtgttccggtcactgttggagggctctgtctgtgttagctcatttaatccccacaacacctcaatcagatggggctattcttagtcccactttatagataaggaaactgaggcatggaagcacagcttgctcaaggttcacatctagtcagtgacagagcaggtatttaaacctcaggaaataatcagagaaacatgtgtagagggttgtccaaggaaggccacatccagaagcatctcccaggacagttgttgtgtagctcaccctctggactttgtgggtctgggtgttgtttcatgattatagagagagctctgtgaacgtggaggacctgttgtcggcagagacacaaatggccagggcatggctgggcagccgcagtggctcaggcctgtaatcccagcacttcgagaagaccagaggggcagatcatgaggtcagaagttcaagaccagcctggccaacatggtgaaaccccgtctctactaaaaatacaaaaattagccaggtgtggtggtgggcacctgtaatcccagctactcgggaggctgaggcagaagaatcgcttgaacccgggaggtggaggttgcagtgagctgagattgcaccactgcactccagccttggagacagagcgagactctgtctcggaaaaacaaacaaacaagcaaacaaacaaacaaataaatggccagggcaggggagggttgcatattgaataagatgagctctgctggaagcacaggtcagcactaacctgcttcctctctctctgcaggtgccttggcatctcccaatggggtggctttgctctgggctcctgttccctgtgagctgcctggtcctgctgcaggtggcaagctctggtaagtcaccacttctcaatcattcatttgttggctattaatggcgtgccagggtcctgcagtatgtcacctggccttatggagattacactgcagtgggaggggacagccaatgacaagtggccctgattatcagtaaattctaaagattgttagaaagtgatgggagccgggtgcagtggctcacacctgtaatcccagcacttcaggaggccgaggcaggaggatcgcttgagcccaggagttcgaggtcagcttgggcaacatagggagaccttgtctctacaaataataaaatattagccaggtgtggcagtgcacgcctgtagccccagctactcaggaggccgaggtgggaggatcccttgaactcaggaggtcaaggctgcagtgaactgtgatcgcgccactccactccagcctgcgtgagaaagtgagaccctgtcaaaaaaaaagagaaggtgatggggaaagaacacagaacagcataagagggggttggggaagctgggtggagtgggggggattgcagttgaaagtagggaagtcagggaaggcctcattgagctgacttggaggaagcgggaaccgtgcagatgtctggggaaggctcattcttggcagagaggccctgcactgagcctggcgggagggttgagcacaggagggaatgtggtggaggagagtgagcagcaggagggagcagtgaaggtcagcaaggtgacagagtggctgaatcaaaaaagaccttgcagtgtttgagcagaggatccatatcatccattatgttccaaaggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaaggtactttggggaggggcgggaatcaggagttagttttggacatgagataagcttggaatatttatttgctatctaagacagctccttaacatggtaagcccttatgcaagttgttgtcagctgagatgggcgtggcactgagcatgggagcatggaggcgcctgagtggtctcatgctcaggtggtttagcaaactcagtgtacatcctgccaattccagtcctgccatggccactgacaagctaggagggcgctgaaaggagaaggaccccgatgtctcctccagcccatccatctcctctctcccattggccaaacccaaccggaaactaaaggccaagggtacccggtgatgaagactgtggtatcagcctcctgagcacagagagggcagaaaggggtggagacaaagaggggcgcagatagtgggcaaatggggaagtggcacttcccctagctcgagggcagaggcttggtgtgatggaatggcactccttaaactgctacatattttccctttaatttggccaagaacaagttgtcaagtttgtgtgagataaaggtgcacttggttcgttcttgtctaatggcccccgcacccatgggtatttcttcagcttccacagtcatcccgacactagctgggaagctccagcagccctggtcctggccccagctctgtgggcgctggccctcaactttgcctgcactgtgcttttgtgctattccccttggtcctgtttgggtgcaagtccccctcacgcattgagttcctgggccgctcaggctgctcctgtgtctccccagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgagtaagcctgcgctggagctggaggtttggggaggttgtgcccaaagggtttgccccaagagtgagctgggtccaggtggtgcgctggagtgcaggatgctgagtatggtttgctgctgtttatatggtgttagaggggaggtcccatctccagggacatgttatgtaagatacagtggagcgcatggtgggagtgttggtccacgtggcacatggatacggctggaatactggactagaccagcagttctcacactttttggtctcaggaccctttttcacacttaaaaatgagtgaggacccaaagggctttggtgtaggtaacacatcattctatgtttacctaattagaacttgcaatgaagaaatggtgtaatttttaaaaaattaaaacaattaaaaattttttttcttactgaaatggaggtctcactgtgttgcccaggctgctctcaaactcctgggctccagtgatcctcctgcctccgcctcccaaagtgctgggattacaagcgtgagccgctgtatccggcccaaaatggagaaattttaagtcccaacaacatgcaagcccgcattcaacaaatcttcagatcaattacatgatcacaggtcatgtagcctctagaaaattccactgtacgccagtgagagagagtgaaaaggcaaataacgtccctgtattatgatgaaaagagttttacctggtgggcccagaccacactttgagaaccactggactagacccttgattgaggagtacggtgttgagagtggagtcctctgtgatggtggatggaccaggacacatggcataggagtcaggtggttccctgggctactccatggtgcacaggatgcttcgttacactggtgcccaggacataatcacgtacacaagacacacagttacggggcagactggggatatacggcacaccagcatgcagcgttcaccagtaaaggtggtattccatgattattctaaggtagatgggctgtgctttgtttccattggcttagtccagggattggcaaactatggcccgtgagccaaatccggcccactgcttgtttttgtaaataaagttttattggaacacactggctgctgtagttgtaacagaaactgcatggccctcctttatgttttttgtttgtttgtttgtttgtttgttttctttgagacagagtttcgctcttgttgcccaggctggagtgcagtggcacaatctcggctcactgcaacctctgcctcccgggttcaagcgattctcctgtctcagcctcccgagtagttgggattaatggtgcctgccaccacacccggctaatttttcgtatttttagtagagaccggttttcatcatgttggccaagctggtctcgaactcctgaactcaggtgatccacccgcctcagcgtcccaaagtgctgggattacaggcatgagccactgagcccggcctcctcctttatcttaattgaaataattcagaaatggaaagtcaaatactgcatgttctcacttataagtaagagttaaataatgtgtacacatgggcattattccatgtaccatggaataacagacattgaagacttgggagggtgggagaggggtgaaggaagagaagttacttaatgggcatagtgtacaccatttgggtgacggacccaccagaaccccagacttcaccactaggcagcatatccagtgagaacagatctgaggcttgccatcaaaattgcacttgtaaggccgggcactgtggtggctcgcggctgtaatcccagccctttgggaggccgaggtgggcagatcacttgaggtcaggagttcgagaccggcctggccaacatggtgaagctccatctctactaaaaatacaacaattaactgggtgtagtggcgcacacctgtaatcccagctactagggaggctgaggcgggagaattgcttgagcccaggaggtggaggttgcagtgagccgagatcacatcactgtactctagcctgggtgacagtgagactttgtctcaggaaaaaaaaacaaaaacaaaaaacaaaaaactcgtaccccctaaatttatacaaataaccaaaaaaaaaaaaaaaaaaggaaattgtgtggcctttgaagtccaaaatattaactatctggcctgttacagaaaaagtttgcagacccctggcctagcccgtgagatgtgggttggctgttaaggtggaacattggaattatcttacgatggccaaactgtgcgatgcagagcttatgttgttctaaattaattagtgccaccggttcttccctttcatgggctttcaggaacaagctaagtcccaggaccagggccggcagctaggcaggtgtgaggagcatccttggtgcatgtggtaagaggctgtggccagcaagagaggcaaccctagtcggctgccccagcacaccctggccgctcccaagcccccagatctgtcctcacatccgtgatcgggaagctggaagagtctgatgcggttcctggaggcatgtcccggacacagctgtggggcccagccagcctacaggtgaccagcctaacccagcccctgtgtctgcagagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatggtgagcagggcggagtgcggcaggggtggctgggtgtgttcccacagctgcctgggctgagggtggggtgggcaggggaggaggtggggtcatagcaacagcaggaggaagccgcctgtattttcccaaatctgatgggattcctgcccctgcctgggcctcagtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacattccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaacatggtaaaaccccgtctctactaaaaatacaaaaaattagtctggcatggtggcaggcgcctgtaatcccagctacttgggaggctgaggtaggagaatggcttgaacccaggagaaggaggttgcagtgagcttagatcatgctactgccctccagcctgggcgacagagtgagattacgtctcaaaaaaataaaaataaataaaaataaaaagattttttaaaaggctgaagtttgggttactttggctcatacactttgccttcactgtagaaaggtggttagtaaagaccaggcgcggtggctcatgcctggaatcccagcactttgggagcccagcgcaggcagatcacttgagccctgggctattgaggctgcagtgagctgggattgtgccactgcactccagcctgggcaacagagtgggaccctgtctcaaaaaagaagaaaaaaagggtaattaataaacactaaagttctatgtagaattttagcaacattattgttattataatcttctttgctatggctctgaatctgtgtggtgctccagaagtatgctatggaggttttgtcgaccaaaaatctgggtggtggctgtggtttgtaggccggggctgggctgggtgatgggggagtcactgcatagatcctcacatagaggccgcttctcccgcagtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatgtgagtgggcatgctttgacgtttttctgtgacctctggggaacagggtgggtgaccagcagaggcccagtccctggagccaggagcctgggaggcaagccctggggctggatagcaaatcccaggagctagagacctggcttctcacctggctctgccctaggcaagtccctttgcttcctggccccccacccctcacatcagagaaggggagttatctctgcatgccgctcctcctctgtaaaggtagggctgtgggccacatctgtgtttcccagtttgggggacacaagtgatcgtaggtggcacattgacagctcacttgaataaccctattattgaagagaataatactgactcaagagacagtgacccgtgtcagttcccttttgaggccaacgggttaaggaggaagtccccatacagctgactcgtttactaattcctcttaatgaagagagcagaggccacaccccaggcttagactttcccaagaaaacaagatcagtttgttggttgttccccatggaagctggtcctgacattcccttcacagtagtgttggtggagtttttgttgttgtttgttttgagacagagtctcactctgtcacccagggtggaacacagtggcgtgatcttggctcactgcaacctccgcctcctgggttctagcgattctcctgcctcagcctcctgagcagccgggactacaggcacctgccaccgtgcccagctaatttttgtatatttagtagagatggggtttcactgcgttggccaggctggtctcaaactcctgacctcagatgatccactcgccttggcctcccaaagtgctgggattacaggtgtgagccaccgcacctggccagtggagttccttcttaagtacatgtattgacatctttaaaaagggcgagaggatttacaggaaactatcaggtcagtaatggcaggggccgtccacagtgggtggctgagtccccctatttttctgctggtgtgcagggaggtcatttcctgccacccatgtttccccaccctgaatccaccttcctcacattcccattggagggacaatctctggacatatgggacctggggtcccacagggctgcaatccaatgcctgctgtgccactcgccagctgtgtgatgttgggcatatcccataacctctttgtgcctcagtttcctcatctgtaacacaggagtgacaagagcacccgcccacagggctatgacagtacaaggtgtgtgatacagatgagctcccctgtttggcccacatgtgtcctaaaagccatgtgccctttctcttgagtgccccaggccacagagatccccatctgcccgctgtcccacacactggtctgtcatttgttccttgaggtttgtgagggccggctctgtgcatcccaggggcccaggctgggcctggttggctctcagggagcaggcacccgccaccttaagctcccatgctggtgtctgtcactgcttcctctcaatctggccaagccaggggtgtcgatttatatctctcaggtctggtttcccctttggcactgggccaggtatggggaaagagcaggaatggggcagttggctcacacagcagaggctcagaaagcggggggcatggggggaaggagtgcacagatgctagagagtggggcaagttttgtttggtcaataaatctccttctcatgccccaggcctgtgcaagacctacagagagtcccaaggatgggctggggggaagagaaaggtaccaccttcagagtccaaagatatgttatttaatattttcatatttctagatctgccttcaggcatggctggatccagcttctaggaacctgtccagctctgcgccctgctttattctgtattggcttcgtttttaggcaggctcttccctcatgtagtggcagatatgcctactagttgctccaggcctacatcccaaagccacagtgggaaaagggttttttttcttgacggttctaataagagtcctaaggctgctgctcagtggcctggcttcgatgctgtgccagcctctgaaccaatcactggctgtgggtggagagagggtgctggtggagggccctgcttgtccagggaggagtcacatacctgcctctagggctgcaggtgggctcagctccatccaaaccagatgaactgaaaataaggcaggagtggcttccccaggggaaactggggaagaggaagcaggactgtgctggctaaaatgccagccaggtttaagacgtggcaccagatgccagtcatgggattggattggtcagcatgcctgggctatggcttaggggtatgttggtgctcagggatgccacaggcctccagataccaggtctgaggcagaagaatgaagtccagcttctcttgtgggtggaacagtggcaactgagataccccatctctcccttcccaagaacagagctgaacataaagaatttagtgattggccagagcttggccacatgctcccctctgatgaatgataggccaggtgatgggattggcacaattggcttagactaatgagggttggccctggagttgcaggcagtggagttctgtcctaagcagtgggcacctaaacccgatggcataaaagctgggcgggtgtccacctgcatctgccacagcactataggcaccaactgtggctcatactgagtgggataaattccagaaagaaacattaggaacttactatagaattttggggctagagctactcattcattcccctagataatttctaggcaaggttccatagtggagggggagttttggcttgggcattgaaggatgcataggagttttctagatggggaaagaagggaacggtagaccaggcagagggaactgcatgataaaaggtttatgggtgtgaaaattcatggaatgtttgaggattatggggttgggggatgtgggaatatgtgtagcgataaagcaccaaacaaagccaaaagtttagttagagccctgaatgcctgcctcataatggtttccatattttatatgcctactatgtgccaggcacattgctcagggtcacacagctggaaatggcagggctgagtttttgttgttgttgttgttgttgagacagagtctcactctatcacccaggctggaatgcaggggcgtgatcatggctcactgcatccttgacttcctgggatcaggtgattctcccacctctgcctcccaggtagctgggactacaggcacaggccaccacgccaggctaattttttgtatttttagtagcgacagggtctcgccatgttgtccgggctggtctggatctcctggcttcaagtgatccccctggctcagcctcccaaggtgctgggattacaggcttgagccaccgcatccagcccagatctgagatttgcacccagtatttgaactcccaagcctgtgctctttttcctcccatggacatttctctcagagatggtctcccaaacacctgtccttcttgttaaaaaacagacaaaccgcaagtagttctttggaagctcagatttctcttttgtttcttagtaaaacatttcccagttcccagctcccttccagggtgtaagatttcttcggtaacttacatctagctgttgcttcttgtttgctcatgtttagaaagaaagacaaaagagagtgagaattttctctcccttccccagtctccccacaactcacaccccaccctcagctccctctgtaataggaaaatctctgaactctctgtagttgctccagcaatcttttggaactttgcttctttcttgtgaaaaaacctccccttggctcactttgcaccaggtttccccaaatgtgcttccaaccacaagcagaaatggagctgccagtaaccaggaagaaactgccgggggctgaggaagaggagagggaggtgcatagccctggatctcgcagggagaggggtgacaggatgagaactcaggttgctcacttgccatcagggtcagtcatgaatatagcgttcatgtatcactttttaaagcttttttggagggtaaaagtaatagttacacaaaataaaaatacaaatggtacaaaaggacttagaatggaaacatgtttctctcccgactccagcctcctgtttttcttcccagagactgaccactgctgtctgtctcttgccagaagggaaagggaggcaaggttagggcaggcagagggcatgtgcatcctttagagagagcttatgtctatacaagcaaatgtgtgtgttcagtcatcgctgtcttagttttctattgctgcataataatggtactaccagcttcacagctttaaacaacacccatttattatctcatagtttctgtggttgggagtctggacatagcttagccaggttctctgctttagagtctcgtgaggctataatcaaggtgtgggatggggctgcagtttcatctgaggctcaattggggaagggtcacttctaagctcatacaatattggtgacattcagtccctggcaggctgttgaactgagagcctcagtttcgtgctggctgttggttgtagttaaccctgaattccttcccatgtgccctttgcaaagccatcaaggcagagagacttgcctagcaagtaggatattacagtcttctgtaatataatcacatccatgaaatcctctatatatcccatcacctttaccatattctgtgggttagaaacaagtagcaggtcctgcccacactcgagaagaccagatgacacaaagatgtgattcaaagtggggatcatcggggccatcttaggtttgtctgcagtgatcactgtgccatctctctctctctcttttttttttttttttttccgagacgaagtcgtcactctgtcacccaggctggagtgcagtggcatgatctcagcttaccacaatctctgcctcccaggttcaaatgattcttctgcctcagcctcctgagtagctgggattacaggtgcccgccaccacacccagctaatttttgtatttttagtagagacagagtttcaccatgttggccaggctggtcttgaactcctcacctcaagtgatccacccacttcggcctcccaaagtgctgggattacaggcatgagccaccatgcccagccccatctctctttaaaaaacaaacaaacaaacaaaaaacataaaaagaagcagagaacacatacacatctgcatcttcccttgtttacttaacaatagatcttggaagtcacttctcagtagaggctaggttgggcagagcattggattctaggccagtgagtttggacttgaccatggagacactaggaagcccatgaaggacagagagagatgcctcgaccctgccagtcctttagaaagatcacccagtgctttttgtataccaaaccctatttgaaatacttacgtatattaacccatttccttatcaccacaaccctgcgggaagggagataggcacttttattatcttcattttgcagatgaggacattgaggtccagagaggttatgtcacttacttaaggtcacacagccaggaagtggtagtagggactcttacccttgttttacagatgagattgaattatctcacgaaaactcagaaaggttaaacaacttgcctaagtaacatacagctaattagtcgaggagcctgacgcatgttgctctagcctggtcacagttacagaggtggcaagcaatggcctgaacaggacgaacaaccaaatacccaggctggtggctcttaaacatggtggggtcagctaacgacagcaaccagggtgggcactggtgcccctcgcccccggctggtgccctaacatctcccttttctctaccagttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcccctgtggccagacacttcccctggctgagtctctgggcttttatatcataggatgcctctaatggcaatcctgccattagatacacctgctgtggtgtatctgccaggtaggcaggctaggctgcagtaacacacaagcccacaatttccatggcttaacactataggaatatatttcttgctcatgtaacaagctaacgtgaatgttgctggtttgtaggtggtttccctccctgtagaaatctggggagtgaggttctttccatcttgtggtgccatcattctccaggacaaagattcttacctacttttgtgtcctggtttcctttggcagcctggtgaagcctatggacctcatttcagaatatttttaaatacataaaatcccagcctgggcaatatagtgaaacccccatctgtacaaaaattagccaggcatggtggcatgcacctgtagtcccaggtactgggaaggctgaggtgggaggatcacttgagcccaggagtttgaggctgcagtgagccgtgatcgtaccactttactcccacctgggtgacagagcaagagcccatctctaaaaataaataaatacaatgaaataaaataaaataaatagaactacagaggaaactaattgtattgaaatgcagttataaaacatttaaacacatttttaatctagagatatatgtgcttctttattaagatctataaataataagttctaggggtagctcgcataaatactgtaatttcaaagtagataagcataaataatactttatgatactgaaattgtgatgtgatatgagaatagctgtgagttttgttttgctggggacaggatcactgatgctgtcattactggggtctcttccctccattctttttttaaaattgtattttattttatttttaaaattttaaaataaatagagacagggtatcactatgttgcccaggctgcttttgacctcctgggctccagtgatcttcccatcttggcttcccaaagtgctgggattacaagtgggagccagtgttcctggccccttcctccattcttaatggaaggagatgctaggtgtgagaggttagggaaagtaaagatgtaatttctttcccatccaagttctcagacccctgaattctacctgcagccatgttggtccatcaaccccaagtgaagaatccctgctctagggccccaccattgtctgtatccagccagcagaagaggcgtgattatggagatcacatctgcttcttgaaagcagacagcccggaagtgggccgcatcacttcctctcaaattctattggtgaaaatggtcacatgactacacatagccacaaaggaggctgggaactttctcacttggaacctacatcccagaaacaactcttttcagtgaggtatcccacaggtctttcgcagtagaaatattgattatctcacataaaatgaagtcttacaaatggacctactgggttttgtacagcagccaagtgatatctcttcccttctgctgtcttcccttctgccgtccttcacatggtggcattgtatccttagacttgccacccatgccctcaggttggccgttgcacactgtcttacataaagcaggaaggaaaggaaaggctgctacgagagagtgtaccttgtgcatctcttttttaatcaggaagcaaacatctttctagaagcttccctagcaaaattccccttacatctcattggccaagactgttacatgttacatggttactgttattacttgctcattgcaaggaagactgggaactcaaatgcctggaaaaaggaacaggataatcgtgattggctcaagccttagggtgggcatggctccctgacaagggagagaggaaaaagctgttgagtgaagaagactgcttcagtttccccatctgtataatgggaggagtaagggctgtcgtgaaaactcaatgaaagaagattcttcaacgtggtaggtgcagtggcagctggcagtaccctgaccctgccaccgcacagccctctcagcattgctcatcctgcactgtggatatcagttgagccacgtgtctcctgccctgggctgtgagctccataggcagggtctccatggctgtatctccagaacccagcacagaaccaggtgcttgggaaagttttgaattgattctcatctgccattggcatggggaagggaactagcttgtatgaaacagataacaatgtatgggaccctcattcattatttcagcaaatatttgctgagttcctcctacatggctagccctgtgctagacactggggaatcggcgatgaacaaagcagatagaaatccccactcttgtggagctgacattctggagggagagacaaaaagcaaacatataaagaaagaaagaaatcacatggatctggatgacagtgagtgctgggaagaaaataaaagcagaggaaggggatggagcgatgggcagggggcaacggtagggagggtgtcggggaaaactttttggagaatgtgacgatgaaagtgaacaaggagaagtcaaccgtgttgagatgatggcagctaatgatgtggacaggccactctgttctgagtgcattatctattgattcatcatgtcatcctcgcaacagccctgcacgatcaattctgtcattaaccccatagtacagatgaggatgcggaggcacagagaagataagggacttgtcctgtgtcacacagcaaggagccatccggctcctaagttggtgcatttgacttctgtgcttccggaaagaaagagcagcaagtttaagatctggaggtggcactgagctttggaggagcagggggcaatgaggtggccggtgtgacgaggactcaatgtgcaagagggagagtggtggggagatgaggtggaggggtggtcggcggtcagatcgtggagggtctcggacgagggtcctgaccctgggtctccagtcctgggaagtggagcccaggctgtaccatggctgacctcagctcatggcttcccctcccacttccagcctacagggagcccttcgagcagcacctcctgctgggcgtcagcgtttcctgcattgtcatcctggccgtctgcctgttgtgctatgtcagcatcaccaagtgagtcctgggcccagtgctgccgagcagtccctctggagtgcagggtggcagggacttgcccctctagtctgcccctttgcagtcctctcagtcaataatacgtatttactgagcagctactacacaccttgagagtagagctgagaacatatcgacaaggaccccacttttttctttttttcttttttttttttttttgagacggagtctcactctgtcacccaggctggagtatagtggcacaatcttgcctaacagtaacctccgcctcccgggttcaagcaattcttctgcctcagcctccagagtagctgggattacaggcgcatgccactatgcccggctaattttttgtatttttggtagagatggggtttcaccatgttggtcaggctggtctcgaactcctgacctcatgatctgcctgcctcagcctcccaaagtgctgggattacaggtgtgagccactgcacccaaccaggactccacatttctaaaaccggcatcctactggggagactgaaaatacatatcaatcacaaacaggtggttttccatagtgacccactctctgaatgcactagaccagggtggaggccagagatcttctggggtgctttttgcaagggggaccaggataaggctctccaaggagggaaaatttgaggggggccctgactggggagaatgagctggccagggataagcaagatggagtcatcccacatccccttacaacactgggtgcctgggcaactgggggcatttgggggcatgtggtaggagccagaggaatttgcgacgattgccctgatggagtcaggagacctgggtttgaatcctggccttggagcttggtagctggcggccgacaagttgctgaaacccctgagcctggggttcctgctttgcagagtgacagtgatggtgagaacatatttcatcagccagaagaggccaaatcacagtaaaggctgagggaggagatgagtggcgagtggctgggaggtggtggaaggagcctcgtttccagagagctcttgccagcccttggaatcatggtgtctcagagcctcagtcctcccatctctgaaatgggactagcaagctcaacctcactaagtcaggattagaggtggctaaggattattaacatgattgatgaaagtgcccactcttggcccagcacacactaggtaggcagggaatgcaaattcccctccatatcttgtcactgatgcctccgagcaaccttggactgatcgccttgctctgagcctcagtttccccatcacctgtacctcttcccactccccatcactatatcccagcatgccagcctctttgctgttctttgtctttggtttcttgttttgttctgttttttagacagggtctcactctgttagccaggctgaagtgcagtggcgcggttacggctcactgcagcctccaattcctgggctaaagagatcctcccatttcaacttccagagcagctgggacaacaggcgcttgccaccacacctggctaattttcttattttaatttaattttattttattttttgggacagagtggagtctcaaaaaccaagctagagtgcagtggtgcgatctcgactcactgcaatctctgcctcccgggttcaagcgattctcctgccttagcctcccgactagctgggattacaggcgtgtgccacgacacccagctaatttttgtatttttagtagagatggggtttcaccatgttggccaggatggtcttgaactcctgacctcaagtgatccacccacctcgttctcccaaggtgctgggtacaggcatgagccactgtgcctggccaattttcttacattttgtagagactggctgtcacttatgtagcccaggctgatcttgaacttctacccctttatctttattcatggcacttattaccatgaatgaatgacctcatataagcatttctttcgtttttttttttttttctttgagatggagtctcatgttgtcccccaggctggagtgcagtggcgcgatctcagctcactgcaacctccgccttccgggttcaagcgattctcctgcctcagcctcctgagtagctgggattgcaggcgcctgccaccatgcctggctaagttttgcatttttagtagagacggtgtttcaccatattggccaggctggtctcgaacttctgacctcaggtgatacacctgccttggcctcccaaagtgctgggattacaggcgtgagccgccatgcctggcctcatataagcatttctgtctccatttatcatccatctttccctcttgaaggtcagtttcaccaaggcaggcatctttgtctcgttcactgttgtagcctcagggccaggcacagtgagtcaaacatagaaggtgctcaataaatatgtgtttatttattgaaaccatgggcagaggctaattcagaagcggtctgaggaccttacctcccagtgatgatgcaccatggccccaggcaggccaggaagagagaagggttgtgtttctccgtaggtcccccagcttcccaggccatcccaggccattccctggtcatttgccctcagctgctctgaaaaagggattgttgaggggaacctagaatcctctctctgcagtttgagtctttcctaatcccctggggtctcattcccactgaggacataggtggcctcctcaggaactctgtgctgggtaacagaatgcgggagtgtgaacctggctctgccacctaccagctgtcactccacctccttgggcctcactctcctcatctgtagaatagggttagcaatagaatccatgtcaccaggttagaatgatgagtcagtggtttgacctccagaaactaatcagcctgatctctgatgccaaataagtattggtgataacgaccacttttatgggaggagcgttcacctgtcaataattcagagatcaacaccttttccttttgtttttcaggattaagaaagaatggtgggatcagattcccaacccagcccgcagccgcctcgtggctataataatccaggatgctcaggtaggagtaggcgtggatgaggacatgtgggactgtgtacatgaagaagtgtggttcagaacacctgggctgttaaggaccttcactggcttctggaatggcaaatagacagtcaggagggttgcaggggagacagaggcagaagccgaatgaggtcattagcagaccagaggctttcccgcccttccccttggcaatcccagcctggggtgggcttctctggggttggtttcctgtttttttccctccccttgggagaatgacccttgggtcatcatcactgtgtcattccctggggaggtgccagtaccagggctagaggccagaaggagtggaggaaggagagggtgacaggctttctgtgtcttcttcttaagcataggaaactgcccccgaagcactagcaaatcccttccgggttctcattggcctgaaatgtatcccacccctaagccaggggtggagtcagcttccccaaggcgatggtcctgtgggtgagtgggtggggtttgcctgagcaagatgagagttctctaggtaggagaaagggggattataggtcctgtctagaagagaaggtctgagggtccttgcttttccagggactctggaatctagtgttggctttgaatcctgactctgccactcactggcagtgtggacttgagcaagttgcttaattctctgagcctcagtttcctcttgtgggttataacagtgtttacctggtaggacagatattggaatttattgagacaatacatataaagtgcatattccagcctcttgcaaataccaagtgccatttatgtatcagttagtgtttgctgtgtaacaaatgaccccgaaatgtagagggttacaacaactttatttagcttatgcttctgcaggctggcatttggggctgggctcagcagtgagggtggcgggggaggctgggctgggctgggctgggcagatctgaattgagctgacccgtccccgtagcctccctccgtgtctgacagttggctttttttttttttttctttttctgagacggagttttgctcttattgcccaggagtgcaatggcatgatcttggctcactgcaacctctgcctcctgggttcaagcaattttcttgcctcagcctcccaagtagctgggattacaggcatgtgccaccacgccaggctaattttgtatttttaatagagatggggtttcttcatgttggtcaggctggtctggaactcctaatatcagatgatccacccacctcagcctcccaaagtgctgggattacaggcgtgagccactgcacccagcctagttggctgacttttacctgggacagtgcaggtgcctgagccatgtgcctctcactctccagcaggccggcccaggcttgtttacagagtggctcagttttcaagggtgggaagtcccaaggcttcttgaggcctaggcgcagcactggcatgatatcacttccatcacattctatgggcccaagcaagtcccagggccagtgtagattcaagggatgggaggagattcagagcactcctctgtggccacttttgccatcgaccacagtccctgtaaatattaggacaatgtaattaattcccaggaatctgaggctcagaaagcgtaagtgacctgttggacttctgatctgtgtgatgtcgaggcttgtaccccttcctgagcattgccgtactccaggccgggctgcaaggccactctgctctttcattggctgtctctgtattttaggggtcacagtgggagaagcggtcccgaggccaggaaccagccaagtgcccgtatgtatctgaacttaggtcacagcctgcatgcattgggaaggtgatagaattggagaggcaagcccctagctccatgtctgccttctcttccctgcattcggtaattgccctgtgacattagccttcaagggacggcaggaggaggggtgttctggaaacgtggactgctggccaagccccctgagtttcactggtgtgtcaggtacatggtgataccccttgggagtgctgttatagttaacaaccagagcagccgtgcctgttgttaaaatcttgacctaattgtatacttgtcggcaaatagccactatcctgaacactcccctccttttttttaatatacaggatctcactctgtggcccaggctggtgtgcagtggtgcgatcatagctcactgcaccttcaaactcctgagctcaagtgatcctcccatcttagcctcccgagtagctgatactacagatgtgcattaccacgcctggctattttaaaaggtttttgcctgtaattccagctactcaggaggctgaggcatgagaatcacttgaacccgggaggcagaggttgcagtgagcgcagattgtgccactgcactccagcctgggcgacagagtgagactcttgtctcaaaaaaaataataccaaaaaaagtttttgtaaagacaagctctcgctgtgttgccccgccactgtggcctccttagcttcttccctggggcctgctggacctttccatactccagaaactaaagggggtccaggaccctgcttcaaccctaggatcccgcatcttttttttttttttttttttttggacgcagggtcttgctgtgtccctcaggctggagtgcagtgattcactgcagcctcaaactcgtgggctcaagtgattctctagcctcagccttctaagtagctgggactacagtcatacaccaacatgcccagctaattttccttttttttaattcttgtagagatgtttgagacggcttgggctctgttgcccaggctgttctcaaactcctgagctcaagcgatcctccctcctcagcctcctaaagtgctgggattacaggcgtgagccaccgcacccggcttccatatcctttctaattggtcatggcttgggataatggtgttgcttttaattatcatcatccataaagactttttcttactcaacagatctgagcttgtatttggtgcccaggacatgtgctgggttcccgaaatcccaaagacacagaccctaccctcagggatttctcattctagcaacatagactgatcaattactgattataacgttagaaggcatgtctgaagtagacagccatcaggacatggtgatttcaggctgggctttgaagaatgaataggagtttttcaagtgtcgaaactgaaccctgaccaacctttgcttttgcagacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacatggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctgtgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttattttttatgtattaaccaaacatacctccagacactgctgtgagtgctgtgtctctgttaactcctggaattcacccatccagaggaaccaggatgcaagaggttaagaaacttgccgtctgggtttgggttccccatacaaggattcaaatagttgatttaggaagtaatcccgggaaaccctgctaaggtagtggggaactgaggcagggaaggacacaaaccaagaaagtgttacctgaaaggggtccagatgcagaccccaaaagagggttcttgaatctcatgcaagaaagaattcagagcgagtccatagagtcagtgaaagcaagttaatgaggaaagtaaaggaataaaagaatggctactccgtagacagagcagccctgagggttgctggctgcctatttttatggttattgattaattatattccaaacaaggggtggattattatgcctcccttttagaccatatagggtaacttcctgatgttgccatggcatttgtaaactgtcatggcgctgttgggagtgtagcagtgaggacaaccagaggtcactcttgttgccatcttggttttggtgggttagagccatcttctttactgcaacctgttttatcagcaaggtctttatgacttgtatcggtgacgacctcctgtctcattctatgactaagaatgccctaacctcccaggaatgcagcccagtaagtctcagcctcattttacccagcccctcttcaaagctccagtttaaataaacctctgacaaaagggtgagttattcaacagattaccagcatgagtaactgatgcttacctgccggggatctctggaagaccatgcatggcacatgcccagttatgcctgcaaaggagagggagctggggtatttgtccaccagctcccatctgtcattggctgagagctgcttccaggagcattaattctccagcacttccagctactccaggaaaaaaaaaattcttcaactgagagttggaggtgttgagagactctggcacaccaagaagacaggaacaggacaccaacagtggctgatgatacactgccaaggtcacacagctagttagcaacagatctatagtggaatccagacagtgtctccatcacccaggctctctgtagtgatctgcgcttcacatccgaggcaggcagagggatggtgtgggccttagatgggaaggctgggaacctgaagctcctatgtctgtatcacttttgcttctctgagtagctgccctgatttcacacttgaggggcttggccattttagattccttcctgctctaggagcctacatactacactggaaatgatggggagctctctacctcacatgcagcctgatgtttgttagaaacacctccttgcgccaggcatgatggctcatggctgtaatcccagcaatttgggaggctgaggcgggtgtatcacttgaggtgaggagttcaagaccagcctggccaatatggtgaaaccctatctctaccaaaaaataaaaaattagccgggtgtggtggtgggtgcctgtaatctcagctacttgggaggctgagttggtagaattgcttcaacctgggacgcggaggttgcagtgagctgagattgtgccattgcactccagcttggatgacagagtgagaccctgtctcaggaaaaaaaaaaaaaaacaaaaaaaaccttgttctaagccaaaatcaatccctttagctgcccaaatcacacagtttacagatggagaaacagttttagagaggaaaagggacttgcccaaagtcacccagagaatggcagagcctgaactagccttctggacttcttgcctccaaaagctctttataataaaatataattttaaataaaaatagttatctgtttagggccaagcaatatgctaagtgccgtccagccactgtgtcatttacgtctccaaacagctctagttgggaggctcaatgattatcccaattttacagataaggaaacaggtccagagaggttgaggattagcctagaaccacacagctaggaaatcctggagccaggatttgaacccgggtctgacctaagagctcccagccgccgtgatatatcagcttatgtcatcctgacacctacgcagatgtcggctcgaatccactttgcctgagcattgtctcagagaaatctaatttaaaaattaggcagcaaatagaaaatatatttgactgctagagatgcaatgggactgggagcccaacaaaggatcttaggcaaaagaaatccaagttgttggcctcagcaactattactgaactggctgggctttgggaagctacagagggatgagaagacctggtggatcaggtgggcccaactcaggctggcccccaccctgcaggaagtaggaaaagtccagggtcataggcccagtgagatgccggctgcgggagtttcagcctccggggctggaccagagggcaggaggggacgcccctgggtagcagcgccagagtgggctgagtggcctgggcccctgcgggggagctttcagagatgttgatttgggggtactccctcagccctgcctttacacagaatttgtgggggatgaggggagggggaaaggggggaggaaggcagtgagtgcatctgaattttttttttttttttacaaaaagtggcttattgcatttttctgattactctatcagcacgtgcagaccttttcctattcagagaaagcctgaagatataaagaggaaagtgaagaaaaaccaccggaaatcccatccccgccccagcatctggcactgtgtgggcgatcacgaaatgagcgcttgtttttgaaggcgtagtatctccgtgaacatccggttgaacaacctttctgactttatttttcccacgaaagttattaattaaaaaacaaaaagcaaaacaccgaaaaaacaaaaaacccagcaagtgtttgagctcccaccacgagggaggcctgacgtcactggatcctcccggcagccgatgaggctgcatgggact

SEQ ID NO: 2 - DNA, 2476 bp, Homo sapiens
atggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctctt

SEQ ID NO: 3 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 4 - DNA, 15 bp, Homo sapiens
ttgcaccactgcact

SEQ ID NO: 5 - DNA, 15 bp, Homo sapiens
ttgcaccgctgcact

SEQ ID NO: 6 - DNA, 15 bp, Homo sapiens
ttttgtgctattccc

SEQ ID NO: 7 - DNA, 15 bp, Homo sapiens
ttttgtgttattccc

SEQ ID NO: 8 - DNA, 15 bp, Homo sapiens
ctgggccgctcaggc

SEQ ID NO: 9 - DNA, 15 bp, Homo sapiens
ctgggcctctcaggc

SEQ ID NO: 10 - DNA, 15 bp, Homo sapiens
taagcctgcgctgga

SEQ ID NO: 11 - DNA, 15 bp, Homo sapiens
taagcctccgctgga

SEQ ID NO: 12 - DNA, 15 bp, Homo sapiens
agaacaacggaggcg

SEQ ID NO: 13 - DNA, 15 bp, Homo sapiens
agaacaatggaggcg

SEQ ID NO: 14 - DNA, 15 bp, Homo sapiens
cggaggcgcggggtg

SEQ ID NO: 15 - DNA, 15 bp, Homo sapiens
cggaggcacggggtg

SEQ ID NO: 16 - DNA, 15 bp, Homo sapiens
gtgcggataactata

SEQ ID NO: 17 - DNA, 15 bp, Homo sapiens
gtgcggacaactata

SEQ ID NO: 18 - DNA, 15 bp, Homo sapiens
cggagtgcggcaggg

SEQ ID NO: 19 - DNA, 15 bp, Homo sapiens
cggagtgtggcaggg

SEQ ID NO: 20 - DNA, 15 bp, Homo sapiens
gcctgggctgagggt

SEQ ID NO: 21 - DNA, 15 bp, Homo sapiens
gcctgggttgagggt

SEQ ID NO: 22 - DNA, 15 bp, Homo sapiens
tggggtgggcagggg

SEQ ID NO: 23 - DNA, 15 bp, Homo sapiens
tggggtgagcagggg

SEQ ID NO: 24 - DNA, 15 bp, Homo sapiens
ttctcccgcagtgaa

SEQ ID NO: 25 - DNA, 15 bp, Homo sapiens
ttctcccacagtgaa

SEQ ID NO: 26 - DNA, 15 bp, Homo sapiens
gtgaaaacgacccgg

SEQ ID NO: 27 - DNA, 15 bp, Homo sapiens
gtgaaaatgacccgg

SEQ ID NO: 28 - DNA, 15 bp, Homo sapiens
ggcaagccctggggc

SEQ ID NO: 29 - DNA, 15 bp, Homo sapiens
ggcaagctctggggc

SEQ ID NO: 30 - DNA, 15 bp, Homo sapiens
gccctggggctggat

SEQ ID NO: 31 - DNA, 15 bp, Homo sapiens
gccctggagctggat

SEQ ID NO: 32 - DNA, 15 bp, Homo sapiens
atagcaaatcccagg

SEQ ID NO: 33 - DNA, 15 bp, Homo sapiens
atagcaattcccagg

SEQ ID NO: 34 - DNA, 15 bp, Homo sapiens
gctctgccctaggca

SEQ ID NO: 35 - DNA, 15 bp, Homo sapiens
gctctgcactaggca

SEQ ID NO: 36 - DNA, 15 bp, Homo sapiens
cccccacccctcaca

SEQ ID NO: 37 - DNA, 15 bp, Homo sapiens
cccccactcctcaca

SEQ ID NO: 38 - DNA, 15 bp, Homo sapiens
tccctccgcatcgca

SEQ ID NO: 39 - DNA, 15 bp, Homo sapiens
tccctccacatcgca

SEQ ID NO: 40 - DNA, 15 bp, Homo sapiens
cacctgctgtggtgt

SEQ ID NO: 41 - DNA, 15 bp, Homo sapiens
cacctgccgtggtgt

SEQ ID NO: 42 - DNA, 15 bp, Homo sapiens
atgtctgaagtagac

SEQ ID NO: 43 - DNA, 15 bp, Homo sapiens
atgtctgcagtagac

SEQ ID NO: 44 - DNA, 15 bp, Homo sapiens
tgaccaacctttgct

SEQ ID NO: 45 - DNA, 15 bp, Homo sapiens
tgaccaacttttgct

SEQ ID NO: 46 - DNA, 15 bp, Homo sapiens
cctgttttctggagc

SEQ ID NO: 47 - DNA, 15 bp, Homo sapiens
cctgtttcctggagc

SEQ ID NO: 48 - DNA, 15 bp, Homo sapiens
tggacctgctcggag

SEQ ID NO: 49 - DNA, 15 bp, Homo sapiens
tggaccttctcggag

SEQ ID NO: 50 - DNA, 15 bp, Homo sapiens
agtcatgccttcttc

SEQ ID NO: 51 - DNA, 15 bp, Homo sapiens
agtcatgtcttcttc

SEQ ID NO: 52 - DNA, 15 bp, Homo sapiens
gccttcttccacctt

SEQ ID NO: 53 - DNA, 15 bp, Homo sapiens
gccttctcccacctt

SEQ ID NO: 54 - DNA, 15 bp, Homo sapiens
cagcccccgtctcgg

SEQ ID NO: 55 - DNA, 15 bp, Homo sapiens
cagcccctgtctcgg

SEQ ID NO: 56 - DNA, 15 bp, Homo sapiens
ggagtttgtacatgc

SEQ ID NO: 57 - DNA, 15 bp, Homo sapiens
ggagtttatacatgc

SEQ ID NO: 58 - DNA, 15 bp, Homo sapiens
cagctccccagagca

SEQ ID NO: 59 - DNA, 15 bp, Homo sapiens
cagctcctcagagca

SEQ ID NO: 60 - DNA, 15 bp, Homo sapiens
agacaggtcctcgcc

SEQ ID NO: 61 - DNA, 15 bp, Homo sapiens
agacagggcctcgcc

SEQ ID NO: 62 - DNA, 15 bp, Homo sapiens
ctgcccctggcaatg

SEQ ID NO: 63 - DNA, 15 bp, Homo sapiens
ctgcccccggcaatg

SEQ ID NO: 64 - DNA, 15 bp, Homo sapiens
aggtgcatgtcctct

SEQ ID NO: 65 - DNA, 15 bp, Homo sapiens
aggtgcacgtcctct

SEQ ID NO: 66 - DNA, 15 bp, Homo sapiens
gtgcatgtcctcttg

SEQ ID NO: 67 - DNA, 15 bp, Homo sapiens
gtgcatgccctcttg

SEQ ID NO: 68 - DNA, 15 bp, Homo sapiens
ggcttatccatgcct

SEQ ID NO: 69 - DNA, 15 bp, Homo sapiens
ggcttattcatgcct

SEQ ID NO: 70 - DNA, 15 bp, Homo sapiens
agccaggctggcaga

SEQ ID NO: 71 - DNA, 15 bp, Homo sapiens
agccagggtggcaga

SEQ ID NO: 72 - DNA, 15 bp, Homo sapiens
ggcccacatggaggc

SEQ ID NO: 73 - DNA, 15 bp, Homo sapiens
ggcccacgtggaggc

SEQ ID NO: 74 - DNA, 15 bp, Homo sapiens
taacacagccatcaa

SEQ ID NO: 75 - DNA, 15 bp, Homo sapiens
taacacaaccatcaa

SEQ ID NO: 76 - DNA, 15 bp, Homo sapiens
taatgctcgtctgtg

SEQ ID NO: 77 - DNA, 15 bp, Homo sapiens
taatgcttgtctgtg

SEQ ID NO: 78 - DNA, 15 bp, Homo sapiens
acttgccgtctgggt

SEQ ID NO: 79 - DNA, 15 bp, Homo sapiens
acttgccatctgggt

SEQ ID NO: 80 - DNA, 15 bp, Homo sapiens
ctgagattgcaccac

SEQ ID NO: 81 - DNA, 15 bp, Homo sapiens
ggctggagtgcagtg

SEQ ID NO: 82 - DNA, 15 bp, Homo sapiens
ctgagattgcaccgc

SEQ ID NO: 83 - DNA, 15 bp, Homo sapiens
ggctggagtgcagcg

SEQ ID NO: 84 - DNA, 15 bp, Homo sapiens
ctgtgcttttgtgct

SEQ ID NO: 85 - DNA, 15 bp, Homo sapiens
accaaggggaatagc

SEQ ID NO: 86 - DNA, 15 bp, Homo sapiens
ctgtgcttttgtgtt

SEQ ID NO: 87 - DNA, 15 bp, Homo sapiens
accaaggggaataac

SEQ ID NO: 88 - DNA, 15 bp, Homo sapiens
gagttcctgggccgc

SEQ ID NO: 89 - DNA, 15 bp, Homo sapiens
ggagcagcctgagcg

SEQ ID NO: 90 - DNA, 15 bp, Homo sapiens
gagttcctgggcctc

SEQ ID NO: 91 - DNA, 15 bp, Homo sapiens
ggagcagcctgagag

SEQ ID NO: 92 - DNA, 15 bp, Homo sapiens
tccgagtaagcctgc

SEQ ID NO: 93 - DNA, 15 bp, Homo sapiens
tccagctccagcgca

SEQ ID NO: 94 - DNA, 15 bp, Homo sapiens
tccgagtaagcctcc

SEQ ID NO: 95 - DNA, 15 bp, Homo sapiens
tccagctccagcgga

SEQ ID NO: 96 - DNA, 15 bp, Homo sapiens
tccctgagaacaacg

SEQ ID NO: 97 - DNA, 15 bp, Homo sapiens
accccgcgcctccgt

SEQ ID NO: 98 - DNA, 15 bp, Homo sapiens
tccctgagaacaatg

SEQ ID NO: 99 - DNA, 15 bp, Homo sapiens
accccgcgcctccat

SEQ ID NO: 100 - DNA, 15 bp, Homo sapiens
gaacaacggaggcgc

SEQ ID NO: 101 - DNA, 15 bp, Homo sapiens
cacacgcaccccgcg

SEQ ID NO: 102 - DNA, 15 bp, Homo sapiens
gaacaacggaggcac

SEQ ID NO: 103 - DNA, 15 bp, Homo sapiens
cacacgcaccccgtg

SEQ ID NO: 104 - DNA, 15 bp, Homo sapiens
tggtcagtgcggata

SEQ ID NO: 105 - DNA, 15 bp, Homo sapiens
ccagtgtatagttat

SEQ ID NO: 106 - DNA, 15 bp, Homo sapiens
tggtcagtgcggaca

SEQ ID NO: 107 - DNA, 15 bp, Homo sapiens
ccagtgtatagttgt

SEQ ID NO: 108 - DNA, 15 bp, Homo sapiens
gcagggcggagtgcg

SEQ ID NO: 109 - DNA, 15 bp, Homo sapiens
agccacccctgccgc

SEQ ID NO: 110 - DNA, 15 bp, Homo sapiens
gcagggcggagtgtg

SEQ ID NO: 111 - DNA, 15 bp, Homo sapiens
agccacccctgccac

SEQ ID NO: 112 - DNA, 15 bp, Homo sapiens
acagctgcctgggct

SEQ ID NO: 113 - DNA, 15 bp, Homo sapiens
caccccaccctcagc

SEQ ID NO: 114 - DNA, 15 bp, Homo sapiens
acagctgcctgggtt

SEQ ID NO: 115 - DNA, 15 bp, Homo sapiens
caccccaccctcaac

SEQ ID NO: 116 - DNA, 15 bp, Homo sapiens
tgagggtggggtggg

SEQ ID NO: 117 - DNA, 15 bp, Homo sapiens
cctcctcccctgccc

SEQ ID NO: 118 - DNA, 15 bp, Homo sapiens
tgagggtggggtgag

SEQ ID NO: 119 - DNA, 15 bp, Homo sapiens
cctcctcccctgctc

SEQ ID NO: 120 - DNA, 15 bp, Homo sapiens
ggccgcttctcccgc

SEQ ID NO: 121 - DNA, 15 bp, Homo sapiens
ctgggtttcactgcg

SEQ ID NO: 122 - DNA, 15 bp, Homo sapiens
ggccgcttctcccac

SEQ ID NO: 123 - DNA, 15 bp, Homo sapiens
ctgggtttcactgtg

SEQ ID NO: 124 - DNA, 15 bp, Homo sapiens
tttggagtgaaaacg

SEQ ID NO: 125 - DNA, 15 bp, Homo sapiens
catctgccgggtcgt

SEQ ID NO: 126 - DNA, 15 bp, Homo sapiens
tttggagtgaaaatg

SEQ ID NO: 127 - DNA, 15 bp, Homo sapiens
catctgccgggtcat

SEQ ID NO: 128 - DNA, 15 bp, Homo sapiens
ctgggaggcaagccc

SEQ ID NO: 129 - DNA, 15 bp, Homo sapiens
tatccagccccaggg

SEQ ID NO: 130 - DNA, 15 bp, Homo sapiens
ctgggaggcaagctc

SEQ ID NO: 131 - DNA, 15 bp, Homo sapiens
tatccagccccagag

SEQ ID NO: 132 - DNA, 15 bp, Homo sapiens
aggcaagccctgggg

SEQ ID NO: 133 - DNA, 15 bp, Homo sapiens
tttgctatccagccc

SEQ ID NO: 134 - DNA, 15 bp, Homo sapiens
aggcaagccctggag

SEQ ID NO: 135 - DNA, 15 bp, Homo sapiens
tttgctatccagctc

SEQ ID NO: 136 - DNA, 15 bp, Homo sapiens
ggctggatagcaaat

SEQ ID NO: 137 - DNA, 15 bp, Homo sapiens
ctagctcctgggatt

SEQ ID NO: 138 - DNA, 15 bp, Homo sapiens
ggctggatagcaatt

SEQ ID NO: 139 - DNA, 15 bp, Homo sapiens
ctagctcctgggaat

SEQ ID NO: 140 - DNA, 15 bp, Homo sapiens
cacctggctctgccc

SEQ ID NO: 141 - DNA, 15 bp, Homo sapiens
gggacttgcctaggg

SEQ ID NO: 142 - DNA, 15 bp, Homo sapiens
cacctggctctgcac

SEQ ID NO: 143 - DNA, 15 bp, Homo sapiens
gggacttgcctagtg

SEQ ID NO: 144 - DNA, 15 bp, Homo sapiens
cctggccccccaccc

SEQ ID NO: 145 - DNA, 15 bp, Homo sapiens
ctctgatgtgagggg

SEQ ID NO: 146 - DNA, 15 bp, Homo sapiens
cctggccccccactc

SEQ ID NO: 147 - DNA, 15 bp, Homo sapiens
ctctgatgtgaggag

SEQ ID NO: 148 - DNA, 15 bp, Homo sapiens
gaaccctccctccgc

SEQ ID NO: 149 - DNA, 15 bp, Homo sapiens
gctggctgcgatgcg

SEQ ID NO: 150 - DNA, 15 bp, Homo sapiens
gaaccctccctccac

SEQ ID NO: 151 - DNA, 15 bp, Homo sapiens
gctggctgcgatgtg

SEQ ID NO: 152 - DNA, 15 bp, Homo sapiens
tagatacacctgctg

SEQ ID NO: 153 - DNA, 15 bp, Homo sapiens
gcagatacaccacag

SEQ ID NO: 154 - DNA, 15 bp, Homo sapiens
tagatacacctgccg

SEQ ID NO: 155 - DNA, 15 bp, Homo sapiens
gcagatacaccacgg

SEQ ID NO: 156 - DNA, 15 bp, Homo sapiens
gaaggcatgtctgaa

SEQ ID NO: 157 - DNA, 15 bp, Homo sapiens
atggctgtctacttc

SEQ ID NO: 158 - DNA, 15 bp, Homo sapiens
gaaggcatgtctgca

SEQ ID NO: 159 - DNA, 15 bp, Homo sapiens
atggctgtctactgc

SEQ ID NO: 160 - DNA, 15 bp, Homo sapiens
gaaccctgaccaacc

SEQ ID NO: 161 - DNA, 15 bp, Homo sapiens
tgcaaaagcaaagga

SEQ ID NO: 162 - DNA, 15 bp, Homo sapiens
gaaccctgaccaatc

SEQ ID NO: 163 - DNA, 15 bp, Homo sapiens
tgcaaaagcaaagaa

SEQ ID NO: 164 - DNA, 15 bp, Homo sapiens
tcttgccctgttttc

SEQ ID NO: 165 - DNA, 15 bp, Homo sapiens
tgttgtgctccagaa

SEQ ID NO: 166 - DNA, 15 bp, Homo sapiens
tcttgccctgtttcc

SEQ ID NO: 167 - DNA, 15 bp, Homo sapiens
tgttgtgctccagga

SEQ ID NO: 168 - DNA, 15 bp, Homo sapiens
tgttcctggacctgc

SEQ ID NO: 169 - DNA, 15 bp, Homo sapiens
tctcctctccgagca

SEQ ID NO: 170 - DNA, 15 bp, Homo sapiens
tgttcctggaccttc

SEQ ID NO: 171 - DNA, 15 bp, Homo sapiens
tctcctctccgagaa

SEQ ID NO: 172 - DNA, 15 bp, Homo sapiens
tgggggagtcatgcc

SEQ ID NO: 173 - DNA, 15 bp, Homo sapiens
aaggtggaagaaggc

SEQ ID NO: 174 - DNA, 15 bp, Homo sapiens
tgggggagtcatgtc

SEQ ID NO: 175 - DNA, 15 bp, Homo sapiens
aaggtggaagaagac

SEQ ID NO: 176 - DNA, 15 bp, Homo sapiens
agtcatgccttcttc

SEQ ID NO: 177 - DNA, 15 bp, Homo sapiens
ttcccgaaggtggaa

SEQ ID NO: 178 - DNA, 15 bp, Homo sapiens
agtcatgccttctcc

SEQ ID NO: 179 - DNA, 15 bp, Homo sapiens
ttcccgaaggtggga

SEQ ID NO: 180 - DNA, 15 bp, Homo sapiens
cagctgcagcccccg

SEQ ID NO: 181 - DNA, 15 bp, Homo sapiens
tgggggccgagacgg

SEQ ID NO: 182 - DNA, 15 bp, Homo sapiens
cagctgcagcccctg

SEQ ID NO: 183 - DNA, 15 bp, Homo sapiens
tgggggccgagacag

SEQ ID NO: 184 - DNA, 15 bp, Homo sapiens
ctatcaggagtttgt

SEQ ID NO: 185 - DNA, 15 bp, Homo sapiens
tccaccgcatgtaca

SEQ ID NO: 186 - DNA, 15 bp, Homo sapiens
ctatcaggagtttat

SEQ ID NO: 187 - DNA, 15 bp, Homo sapiens
tccaccgcatgtata

SEQ ID NO: 188 - DNA, 15 bp, Homo sapiens
cccaagcagctcccc

SEQ ID NO: 189 - DNA, 15 bp, Homo sapiens
cccaggtgctctggg

SEQ ID NO: 190 - DNA, 15 bp, Homo sapiens
cccaagcagctcctc

SEQ ID NO: 191 - DNA, 15 bp, Homo sapiens
cccaggtgctctgag

SEQ ID NO: 192 - DNA, 15 bp, Homo sapiens
ctgtggagacaggtc

SEQ ID NO: 193 - DNA, 15 bp, Homo sapiens
gtagggggcgaggac

SEQ ID NO: 194 - DNA, 15 bp, Homo sapiens
ctgtggagacagggc

SEQ ID NO: 195 - DNA, 15 bp, Homo sapiens
gtagggggcgaggcc

SEQ ID NO: 196 - DNA, 15 bp, Homo sapiens
tccatcctgcccctg

SEQ ID NO: 197 - DNA, 15 bp, Homo sapiens
tctgagcattgccag

SEQ ID NO: 198 - DNA, 15 bp, Homo sapiens
tccatcctgcccccg

SEQ ID NO: 199 - DNA, 15 bp, Homo sapiens
tctgagcattgccgg

SEQ ID NO: 200 - DNA, 15 bp, Homo sapiens
tctcttaggtgcatg

SEQ ID NO: 201 - DNA, 15 bp, Homo sapiens
gcaacaagaggacat

SEQ ID NO: 202 - DNA, 15 bp, Homo sapiens
tctcttaggtgcacg

SEQ ID NO: 203 - DNA, 15 bp, Homo sapiens
gcaacaagaggacgt

SEQ ID NO: 204 - DNA, 15 bp, Homo sapiens
tcttaggtgcatgtc

SEQ ID NO: 205 - DNA, 15 bp, Homo sapiens
cagcaacaagaggac

SEQ ID NO: 206 - DNA, 15 bp, Homo sapiens
tcttaggtgcatgcc

SEQ ID NO: 207 - DNA, 15 bp, Homo sapiens
cagcaacaagagggc

SEQ ID NO: 208 - DNA, 15 bp, Homo sapiens
gactagggcttatcc

SEQ ID NO: 209 - DNA, 15 bp, Homo sapiens
tttcccaggcatgga

SEQ ID NO: 210 - DNA, 15 bp, Homo sapiens
gactagggcttattc

SEQ ID NO: 211 - DNA, 15 bp, Homo sapiens
tttcccaggcatgaa

SEQ ID NO: 212 - DNA, 15 bp, Homo sapiens
gaaggcagccaggct

SEQ ID NO: 213 - DNA, 15 bp, Homo sapiens
tggaaatctgccagc

SEQ ID NO: 214 - DNA, 15 bp, Homo sapiens
gaaggcagccagggt

SEQ ID NO: 215 - DNA, 15 bp, Homo sapiens
tggaaatctgccacc

SEQ ID NO: 216 - DNA, 15 bp, Homo sapiens
gatcatggcccacat

SEQ ID NO: 217 - DNA, 15 bp, Homo sapiens
aggtgggcctccatg

SEQ ID NO: 218 - DNA, 15 bp, Homo sapiens
gatcatggcccacgt

SEQ ID NO: 219 - DNA, 15 bp, Homo sapiens
aggtgggcctccacg

SEQ ID NO: 220 - DNA, 15 bp, Homo sapiens
agaaactaacacagc

SEQ ID NO: 221 - DNA, 15 bp, Homo sapiens
attcccttgatggct

SEQ ID NO: 222 - DNA, 15 bp, Homo sapiens
agaaactaacacaac

SEQ ID NO: 223 - DNA, 15 bp, Homo sapiens
attcccttgatggtt

SEQ ID NO: 224 - DNA, 15 bp, Homo sapiens
gttgagtaatgctcg

SEQ ID NO: 225 - DNA, 15 bp, Homo sapiens
aaaacacacagacga

SEQ ID NO: 226 - DNA, 15 bp, Homo sapiens
gttgagtaatgcttg

SEQ ID NO: 227 - DNA, 15 bp, Homo sapiens
aaaacacacagacaa

SEQ ID NO: 228 - DNA, 15 bp, Homo sapiens
taagaaacttgccgt

SEQ ID NO: 229 - DNA, 15 bp, Homo sapiens
acccaaacccagacg

SEQ ID NO: 230 - DNA, 15 bp, Homo sapiens
taagaaacttgccat

SEQ ID NO: 231 - DNA, 15 bp, Homo sapiens
acccaaacccagatg

SEQ ID NO: 232 - DNA, 10 bp, Homo sapiens
agattgcacc

SEQ ID NO: 233 - DNA, 10 bp, Homo sapiens
tggagtgcag

SEQ ID NO: 234 - DNA, 10 bp, Homo sapiens
tgcttttgtg

SEQ ID NO: 235 - DNA, 10 bp, Homo sapiens
aaggggaata

SEQ ID NO: 236 - DNA, 10 bp, Homo sapiens
ttcctgggcc

SEQ ID NO: 237 - DNA, 10 bp, Homo sapiens
gcagcctgag

SEQ ID NO: 238 - DNA, 10 bp, Homo sapiens
gagtaagcct

SEQ ID NO: 239 - DNA, 10 bp, Homo sapiens
agctccagcg

SEQ ID NO: 240 - DNA, 10 bp, Homo sapiens
ctgagaacaa

SEQ ID NO: 241 - DNA, 10 bp, Homo sapiens
ccgcgcctcc

SEQ ID NO: 242 - DNA, 10 bp, Homo sapiens
caacggaggc

SEQ ID NO: 243 - DNA, 10 bp, Homo sapiens
acgcaccccg

SEQ ID NO: 244 - DNA, 10 bp, Homo sapiens
tcagtgcgga

SEQ ID NO: 245 - DNA, 10 bp, Homo sapiens
gtgtatagtt

SEQ ID NO: 246 - DNA, 10 bp, Homo sapiens
gggcggagtg

SEQ ID NO: 247 - DNA, 10 bp, Homo sapiens
cacccctgcc

SEQ ID NO: 248 - DNA, 10 bp, Homo sapiens
gctgcctggg

SEQ ID NO: 249 - DNA, 10 bp, Homo sapiens
cccaccctca

SEQ ID NO: 250 - DNA, 10 bp, Homo sapiens
gggtggggtg

SEQ ID NO: 251 - DNA, 10 bp, Homo sapiens
cctcccctgc

SEQ ID NO: 252 - DNA, 10 bp, Homo sapiens
cgcttctccc

SEQ ID NO: 253 - DNA, 10 bp, Homo sapiens
ggtttcactg

SEQ ID NO: 254 - DNA, 10 bp, Homo sapiens
ggagtgaaaa

SEQ ID NO: 255 - DNA, 10 bp, Homo sapiens
ctgccgggtc

SEQ ID NO: 256 - DNA, 10 bp, Homo sapiens
ggaggcaagc

SEQ ID NO: 257 - DNA, 10 bp, Homo sapiens
ccagccccag

SEQ ID NO: 258 - DNA, 10 bp, Homo sapiens
caagccctgg

SEQ ID NO: 259 - DNA, 10 bp, Homo sapiens
gctatccagc

SEQ ID NO: 260 - DNA, 10 bp, Homo sapiens
tggatagcaa

SEQ ID NO: 261 - DNA, 10 bp, Homo sapiens
gctcctggga

SEQ ID NO: 262 - DNA, 10 bp, Homo sapiens
ctggctctgc

SEQ ID NO: 263 - DNA, 10 bp, Homo sapiens
acttgcctag

SEQ ID NO: 264 - DNA, 10 bp, Homo sapiens
ggccccccac

SEQ ID NO: 265 - DNA, 10 bp, Homo sapiens
tgatgtgagg

SEQ ID NO: 266 - DNA, 10 bp, Homo sapiens
ccctccctcc

SEQ ID NO: 267 - DNA, 10 bp, Homo sapiens
ggctgcgatg

SEQ ID NO: 268 - DNA, 10 bp, Homo sapiens
atacacctgc

SEQ ID NO: 269 - DNA, 10 bp, Homo sapiens
gatacaccac

SEQ ID NO: 270 - DNA, 10 bp, Homo sapiens
ggcatgtctg

SEQ ID NO: 271 - DNA, 10 bp, Homo sapiens
gctgtctact

SEQ ID NO: 272 - DNA, 10 bp, Homo sapiens
ccctgaccaa

SEQ ID NO: 273 - DNA, 10 bp, Homo sapiens
aaaagcaaag

SEQ ID NO: 274 - DNA, 10 bp, Homo sapiens
tgccctgttt

SEQ ID NO: 275 - DNA, 10 bp, Homo sapiens
tgtgctccag

SEQ ID NO: 276 - DNA, 10 bp, Homo sapiens
tcctggacct

SEQ ID NO: 277 - DNA, 10 bp, Homo sapiens
cctctccgag

SEQ ID NO: 278 - DNA, 10 bp, Homo sapiens
gggagtcatg

SEQ ID NO: 279 - DNA, 10 bp, Homo sapiens
gtggaagaag

SEQ ID NO: 280 - DNA, 10 bp, Homo sapiens
catgccttct

SEQ ID NO: 281 - DNA, 10 bp, Homo sapiens
ccgaaggtgg

SEQ ID NO: 282 - DNA, 10 bp, Homo sapiens
ctgcagcccc

SEQ ID NO: 283 - DNA, 10 bp, Homo sapiens
gggccgagac

SEQ ID NO: 284 - DNA, 10 bp, Homo sapiens
tcaggagttt

SEQ ID NO: 285 - DNA, 10 bp, Homo sapiens
accgcatgta

SEQ ID NO: 286 - DNA, 10 bp, Homo sapiens
aagcagctcc

SEQ ID NO: 287 - DNA, 10 bp, Homo sapiens
aggtgctctg

SEQ ID NO: 288 - DNA, 10 bp, Homo sapiens
tggagacagg

SEQ ID NO: 289 - DNA, 10 bp, Homo sapiens
gggggcgagg

SEQ ID NO: 290 - DNA, 10 bp, Homo sapiens
atcctgcccc

SEQ ID NO: 291 - DNA, 10 bp, Homo sapiens
gagcattgcc

SEQ ID NO: 292 - DNA, 10 bp, Homo sapiens
cttaggtgca

SEQ ID NO: 293 - DNA, 10 bp, Homo sapiens
acaagaggac

SEQ ID NO: 294 - DNA, 10 bp, Homo sapiens
taggtgcatg

SEQ ID NO: 295 - DNA, 10 bp, Homo sapiens
caacaagagg

SEQ ID NO: 296 - DNA, 10 bp, Homo sapiens
tagggcttat

SEQ ID NO: 297 - DNA, 10 bp, Homo sapiens
cccaggcatg

SEQ ID NO: 298 - DNA, 10 bp, Homo sapiens
ggcagccagg

SEQ ID NO: 299 - DNA, 10 bp, Homo sapiens
aaatctgcca

SEQ ID NO: 300 - DNA, 10 bp, Homo sapiens
catggcccac

SEQ ID NO: 301 - DNA, 10 bp, Homo sapiens
tgggcctcca

SEQ ID NO: 302 - DNA, 10 bp, Homo sapiens
aactaacaca

SEQ ID NO: 303 - DNA, 10 bp, Homo sapiens
cccttgatgg

SEQ ID NO: 304 - DNA, 10 bp, Homo sapiens
gagtaatgct

SEQ ID NO: 305 - DNA, 10 bp, Homo sapiens
acacacagac

SEQ ID NO: 306 - DNA, 10 bp, Homo sapiens
gaaacttgcc

SEQ ID NO: 307 - DNA, 10 bp, Homo sapiens
caaacccaga

SEQ ID NO: 308 - DNA, 22 bp, Homo sapiens
ccacagtcatcccgacactagc

SEQ ID NO: 309 - DNA, 22 bp, Homo sapiens
tattccagccgtatccatgtgc

SEQ ID NO: 310 - DNA, 22 bp, Homo sapiens
ccttggtgcatgtggtaagagg

SEQ ID NO: 311 - DNA, 23 bp, Homo sapiens
tttcaaaggtgggaggactgagg

SEQ ID NO: 312 - DNA, 20 bp, Homo sapiens
gcagtgagctgggattgtgc

SEQ ID NO: 313 - DNA, 23 bp, Homo sapiens
aactccccttctctgatgtgagg

SEQ ID NO: 314 - DNA, 23 bp, Homo sapiens
tcacagttacagaggtggcaagc

SEQ ID NO: 315 - DNA, 22 bp, Homo sapiens
ctgcctacctggcagatacacc

SEQ ID NO: 316 - DNA, 22 bp, Homo sapiens
agctgtcactccacctccttgg

SEQ ID NO: 317 - DNA, 24 bp, Homo sapiens
aaagcctctggtctgctaatgacc

SEQ ID NO: 318 - DNA, 22 bp, Homo sapiens
gggaggagattcagagcactcc

SEQ ID NO: 319 - DNA, 22 bp, Homo sapiens
cagtccacgtttccagaacacc

SEQ ID NO: 320 - DNA, 21 bp, Homo sapiens
ggcttgggataatggtgttgc

SEQ ID NO: 321 - DNA, 23 bp, Homo sapiens
tacttcccgaaggtggaagaagg

SEQ ID NO: 322 - DNA, 24 bp, Homo sapiens
cagtggagatcagcaagacagtcc

SEQ ID NO: 323 - DNA, 22 bp, Homo sapiens
gggcatctcgggttctacttcc

SEQ ID NO: 324 - DNA, 23 bp, Homo sapiens
gggaagtacgagtgctcacatgc

SEQ ID NO: 325 - DNA, 23 bp, Homo sapiens
cttatacccctcttccccactgc

SEQ ID NO: 326 - DNA, 21 bp, Homo sapiens
tctctgagccaaccactgtgc

SEQ ID NO: 327 - DNA, 23 bp, Homo sapiens
ggctgagtagacaatgccactgc

SEQ ID NO: 328 - DNA, 22 bp, Homo sapiens
ctgtgtccccagagaaatgtgg

SEQ ID NO: 329 - DNA, 23 bp, Homo sapiens
gactcagcaacaagaggacatgc

SEQ ID NO: 330 - DNA, 24 bp, Homo sapiens
ggcattgtctactcagcccttacc

SEQ ID NO: 331 - DNA, 20 bp, Homo sapiens
acaagtcgaggtgcccaagg

SEQ ID NO: 332 - DNA, 25 bp, Homo sapiens
cccacatacatgagggtctcttagg

SEQ ID NO: 333 - DNA, 21 bp, Homo sapiens
attctgcctccagcatcaacc

SEQ ID NO: 334 - DNA, 24 bp, Homo sapiens
aacagagcttccttaggttgatgc

SEQ ID NO: 335 - DNA, 23 bp, Homo sapiens
cctcagttccccactaccttagc

SEQ ID NO: 336 - DNA, 18 bp, Homo sapiens
gcgctggccctcaacttt

SEQ ID NO: 337 - DNA, 20 bp, Homo sapiens
gtccctggagatgggacctc

SEQ ID NO: 338 - DNA, 20 bp, Homo sapiens
gcccccagatctgtcctcac

SEQ ID NO: 339 - DNA, 20 bp, Homo sapiens
ggaaaatacaggcggcttcc

SEQ ID NO: 340 - DNA, 22 bp, Homo sapiens
ggctctgaatctgtgtggtgct

SEQ ID NO: 341 - DNA, 20 bp, Homo sapiens
agccaggtgagaagccaggt

SEQ ID NO: 342 - DNA, 20 bp, Homo sapiens
ggcctgaacaggacgaacaa

SEQ ID NO: 343 - DNA, 20 bp, Homo sapiens
ggcaggattgccattagagg

SEQ ID NO: 344 - DNA, 22 bp, Homo sapiens
tgagtcagtggtttgacctcca

SEQ ID NO: 345 - DNA, 20 bp, Homo sapiens
gcctctgtctcccctgcaac

SEQ ID NO: 346 - DNA, 20 bp, Homo sapiens
ccacttttgccatcgaccac

SEQ ID NO: 347 - DNA, 20 bp, Homo sapiens
ctgccgtcccttgaaggcta

SEQ ID NO: 348 - DNA, 22 bp, Homo sapiens
ccctaccctcagggatttctca

SEQ ID NO: 349 - DNA, 20 bp, Homo sapiens
cccattctcctctccgagca

SEQ ID NO: 350 - DNA, 20 bp, Homo sapiens
atcagcgtggtgcgatgtgt

SEQ ID NO: 351 - DNA, 20 bp, Homo sapiens
acccagctctctgggacacg

SEQ ID NO: 352 - DNA, 21 bp, Homo sapiens
gggatgagttcccaagtgcag

SEQ ID NO: 353 - DNA, 20 bp, Homo sapiens
tggcaagcaggcttgagaag

SEQ ID NO: 354 - DNA, 20 bp, Homo sapiens
ccccaacctgagccagaaac

SEQ ID NO: 355 - DNA, 20 bp, Homo sapiens
tgtccacaagggggtctgtg

SEQ ID NO: 356 - DNA, 20 bp, Homo sapiens
ggctagcagtggggaagagg

SEQ ID NO: 357 - DNA, 18 bp, Homo sapiens
attgccaggggcaggatg

SEQ ID NO: 358 - DNA, 20 bp, Homo sapiens
ccctgtcatggccagtcctt

SEQ ID NO: 359 - DNA, 20 bp, Homo sapiens
gcgacccagtgccctctact

SEQ ID NO: 360 - DNA, 22 bp, Homo sapiens
tgcatgtcctcttgttgctgag

SEQ ID NO: 361 - DNA, 20 bp, Homo sapiens
caatgaccaccctccctgaa

SEQ ID NO: 362 - DNA, 19 bp, Homo sapiens
cggctgtcaaggggtgttc

SEQ ID NO: 363 - DNA, 20 bp, Homo sapiens
ccaaacccagacggcaagtt

SEQ ID NO: 364 - DNA, 24 bp, Homo sapiens
aaacccctggactccaagtgatcc

SEQ ID NO: 365 - DNA, 24 bp, Homo sapiens
aagcgattcttctgcctcagcctc

SEQ ID NO: 366 - DNA, 24 bp, Homo sapiens
ggacagttgttgtgtagctcaccc

SEQ ID NO: 367 - DNA, 22 bp, Homo sapiens
ctatgttgcccaagctgacctc

SEQ ID NO: 368 - DNA, 20 bp, Homo sapiens
gccctcaactttgcctgcac

SEQ ID NO: 369 - DNA, 23 bp, Homo sapiens
agtccagtattccagccgtatcc

SEQ ID NO: 370 - DNA, 22 bp, Homo sapiens
tgatcgggaagctggaagagtc

SEQ ID NO: 371 - DNA, 22 bp, Homo sapiens
cgtttcaaaggtgggaggactg

SEQ ID NO: 372 - DNA, 21 bp, Homo sapiens
cgaccaaaaatctgggtggtg

SEQ ID NO: 373 - DNA, 21 bp, Homo sapiens
caggaagcaaagggacttgcc

SEQ ID NO: 374 - DNA, 22 bp, Homo sapiens
tcttaaacatggtggggtcagc

SEQ ID NO: 375 - DNA, 21 bp, Homo sapiens
catggaaattgtgggcttgtg

SEQ ID NO: 376 - DNA, 22 bp, Homo sapiens
atgtgcaagagggagagtggtg

SEQ ID NO: 377 - DNA, 22 bp, Homo sapiens
tgactgagaggactgcaaaggg

SEQ ID NO: 378 - DNA, 24 bp, Homo sapiens
gcctgatctctgatgccaaataag

SEQ ID NO: 379 - DNA, 20 bp, Homo sapiens
tttgccattccagaagccag

SEQ ID NO: 380 - DNA, 23 bp, Homo sapiens
gatctgtgtgatgtcgaggcttg

SEQ ID NO: 381 - DNA, 21 bp, Homo sapiens
gtccacgtttccagaacaccc

SEQ ID NO: 382 - DNA, 22 bp, Homo sapiens
cgaaatcccaaagacacagacc

SEQ ID NO: 383 - DNA, 22 bp, Homo sapiens
gagttgctgaagctgcggtaag

SEQ ID NO: 384 - DNA, 23 bp, Homo sapiens
gaaaaagggagcttctgtgcatc

SEQ ID NO: 385 - DNA, 20 bp, Homo sapiens
aacaaggggacagggactgg

SEQ ID NO: 386 - DNA, 21 bp, Homo sapiens
cagaaacctgggagcagatcc

SEQ ID NO: 387 - DNA, 24 bp, Homo sapiens
gcaacaagaggacatgcacctaag

SEQ ID NO: 388 - DNA, 23 bp, Homo sapiens
aaaggtagaggacatgccaaagc

SEQ ID NO: 389 - DNA, 21 bp, Homo sapiens
ggagcagccaacaactcgttc

SEQ ID NO: 390 - DNA, 23 bp, Homo sapiens
gctcatttaatccccacaacacc

SEQ ID NO: 391 - DNA, 23 bp, Homo sapiens
ccaccacacctggctaatttttg

SEQ ID NO: 392 - DNA, 23 bp, Homo sapiens
tgtagctcaccctctggactttg

SEQ ID NO: 393 - DNA, 20 bp, Homo sapiens
aatatgcaaccctcccctgc

SEQ ID NO: 394 - DNA, 23 bp, Homo sapiens
tgaggcagaagaatcgcttgaac

SEQ ID NO: 395 - DNA, 22 bp, Homo sapiens
acttgtcattggctgtcccctc

SEQ ID NO: 396 - DNA, 21 bp, Homo sapiens
tttgcctgcactgtgcttttg

SEQ ID NO: 397 - DNA, 23 bp, Homo sapiens
ccatactcagcatcctgcactcc

SEQ ID NO: 398 - DNA, 20 bp, Homo sapiens
aacgacagcaaccagggtgg

SEQ ID NO: 399 - DNA, 23 bp, Homo sapiens
cagcaggtgtatctaatggcagg

SEQ ID NO: 400 - DNA, 22 bp, Homo sapiens
agagtggtggggagatgaggtg

SEQ ID NO: 401 - DNA, 21 bp, Homo sapiens
tgcaaaggggcagactagagg

SEQ ID NO: 402 - DNA, 22 bp, Homo sapiens
cgaccacttttatgggaggagc

SEQ ID NO: 403 - DNA, 23 bp, Homo sapiens
ccaggtgttctgaaccacacttc

SEQ ID NO: 404 - DNA, 23 bp, Homo sapiens
tgtgtgatgtcgaggcttgtacc

SEQ ID NO: 405 - DNA, 22 bp, Homo sapiens
gaatgcagggaagagaaggcag

SEQ ID NO: 406 - DNA, 22 bp, Homo sapiens
gccatcaggacatggtgatttc

SEQ ID NO: 407 - DNA, 22 bp, Homo sapiens
tgagcactcgtacttcccgaag

SEQ ID NO: 408 - DNA, 22 bp, Homo sapiens
tgagagcagcagggatgacttc

SEQ ID NO: 409 - DNA, 23 bp, Homo sapiens
aaactcctgatagccactggtgg

SEQ ID NO: 410 - DNA, 20 bp, Homo sapiens
agatcctccgccgaaatgtc

SEQ ID NO: 411 - DNA, 24 bp, Homo sapiens
ttactcttctctgagatgcccgag

SEQ ID NO: 412 - DNA, 22 bp, Homo sapiens
tgggcagtggcattgtctactc

SEQ ID NO: 413 - DNA, 20 bp, Homo sapiens
ttccaggaggtggcatttcc

(19) United States
(12) Patent Application Publication
(10) Pub. No.: US 2003/0078220 A1
(43) Pub. Date: Apr. 24, 2003

(54) Drug target isogenes: polymorphisms in the Interleukin 4 Receptor Alpha gene

(76) Inventors: Anne Chew, Brookline, MA (US); R. Rex Denton, Madison, CT (US); Amy Duda, Milano (IT); Krishnan Nandabalan, Guilford, CT (US); Joel Claiborne Stephens, Guilford, CT (US); Andreas K. Windemuth, Woodbridge, CT (US)

Correspondence Address: Sandra L. Shaner, Genaissance Pharmaceuticals, Inc., Five Science Park, New Haven, CT, 06511, US

(21) Appl. No.: 10/010,802

(22) Filed: Nov. 9, 2001

Related U.S. Application Data
(62) Division of application No. PCT/US00/19094, filed on Jul. 13, 2000.

(51) Int. Cl. A01K 67/00; A61K 48/00; A61K 38/17; C07H 21/04; C12P 21/04; C07K 14/705

(52) U.S. Cl. 514/44; 800/8; 530/350; 536/23.5; 435/69.52; 435/325; 514/12; 435/320.1

(57) ABSTRACT

Polynucleotides comprising one or more of 38 novel single nucleotide polymorphisms in the human Interleukin 4 Receptor Alpha(IL4Rα) gene are described. Compositions and methods for detecting one or more of these polymorphisms are also disclosed. In addition, various genotypes and haplotypes for IL4Rα gene that exist in the population are described.

### RELATED APPLICATIONS

This application is a division of PCT/US00/19094, filed Jul. 13, 2000.

### FIELD OF THE INVENTION

This invention relates to variation in genes that encode pharmaceutically important proteins. In particular, this invention provides genetic variants of the human Interleukin 4 Receptor Alpha (IL4Rα) gene and methods for identifying which variant(s) of this gene is/are possessed by an individual.

### BACKGROUND OF THE INVENTION

Current methods for identifying pharmaceuticals to treat disease often start by identifying, cloning, and expressing an important target protein related to the disease. A determination of whether an agonist or antagonist is needed to produce an effect that may benefit a patient with the disease is then made. Then, vast numbers of compounds are screened against the target protein to find new potential drugs. The desired outcome of this process is a drug that is specific for the target, thereby reducing the incidence of the undesired side effects usually caused by a compound's activity at non-intended targets.

What this approach fails to consider, however, is that natural variability exists in any and every population with respect to a particular protein. A target protein currently used to screen drugs typically is expressed by a gene cloned from an individual who was arbitrarily selected. However, the nucleotide sequence of a particular gene may vary tremendously among individuals. Subtle alteration(s) in the primary nucleotide sequence of a gene encoding a target protein may be manifested as significant variation in expression of or in the structure and/or function of the protein. Such alterations may explain the relatively high degree of uncertainty inherent in treatment of individuals with drugs whose design is based upon a single representative example of the target. For example, it is well-established that some classes of drugs frequently have lower efficacy in some individuals than others, which means such individuals and their physicians must weigh the possible benefit of a larger dosage against a greater risk of side effects. In addition, variable information on the biological function or effects of a particular protein may be due to different scientists unknowingly studying different isoforms of the gene encoding the protein. Thus, information on the type and frequency of genomic variation that exists for pharmaceutically important proteins would be useful.

The organization of single nucleotide variations (polymorphisms) in the primary sequence of a gene into one of the limited number of combinations that exist as units of inheritance is termed a haplotype. Each haplotype therefore contains significantly more information than individual unorganized polymorphisms. Haplotypes provide an accurate measurement of the genomic variation in the two chromosomes of an individual.

It is well-established that many diseases are associated with specific variations in gene sequences. However while there are examples in which individual polymorphisms act as genetic markers for a particular phenotype, in other cases an individual polymorphism may be found in a variety of genomic backgrounds and therefore shows no definitive coupling between the polymorphism and the causative site for the phenotype (Clark A G et al. 1998 Am J Hum Genet 63:595-612; Ulbrecht M et al. 2000 Am J Respir Crit Care Med 161: 469-74). In addition, the marker may be predictive in some populations, but not in other populations (Clark A G et al. 1998 supra). In these instances, a haplotype will provide a superior genetic marker for the phenotype (Clark A G et al. 1998 supra; Ulbrecht M et al. 2000, supra; Ruaño G & Stephens J C Gen Eng News 19 (21), December 1999).

Analysis of the association between each observed haplotype and a particular phenotype permits ranking of each haplotype by its statistical power of prediction for the phenotype. Haplotypes found to be strongly associated with the phenotype can then have that positive association confirmed by alternative methods to minimize false positives. For a gene suspected to be associated with a particular phenotype, if no observed haplotypes for that gene show association with the phenotype of interest, then it may be inferred that variation in the gene has little, if any, involvement with that phenotype (Ruano & Stephens 1999, supra). Thus, information on the observed haplotypes and their frequency of occurrence in various population groups will be useful in a variety of research and clinical applications.

One possible drug target for the treatment of allergies, asthma, and other immune responses is the Interleukin 4 Receptor Alpha (IL4Rα) gene or its encoded product. IL4R is a transmembrane complex composed of two different protein subunits, a 140-kDa high affinity binding subunit named interleukin-4 receptor a (IL-4Rα; also known as CD 124 antigen) and either a gamma-c subunit, which is present in several cytokine receptors, or an interleukin- 13 receptor 1 (IL- 1 3R1) subunit. Both subunits of the IL-4R are required to bind interleukin-4 (IL-4) and to mediate its transcription-activating effects through the tyrosine kinases, Jak1 and Jak3. Upon binding of IL-4 to the IL-4R, Jak 1 and Jak 3 phosphorylate the IL-4Rα subunit, creating binding sites in the cytoplasmic domain for many other proteins, including SOS, Stat-6, c-fes, and src homology phosphatase 1 (SHP-1). Activated Jak proteins also phosphorylate Stat-proteins, which travel into the nucleus and function as transcription factors. Other IL-4 signal pathways exist, but are less well characterized.

IL-4, one of the most important cytokines involved in the allergic response, is produced when cells from the immune system, in particular T cells, are activated in response to an allergen. Regulation of the immune response involves Helper T-cells that differentiate into two subtypes, Th1 and Th2. Th1 cells express interferon-gamma and interluekin-2 (IL-2), and mediate a cell-based immunity, where macrophages and neutrophils are prominently involved. Th1 cells also direct the IgE-producing B cells, as well as mast cells, basophils and eosinophils. Th2 cells produce IL-4, IL-5, IL-6, IL-10 and IL-13. Each Th cell subtype represses the other, so the immune system is forced into differentiation into either a Th1 or Th2 response against an external allergic challenge. In many instances an aberration of this response can render a pathological state such as a Th2 response against. ragweed.

IL-4 induces in B cells the synthesis of IgE type antibodies that recognize specific allergens. IgE binds to receptors on mast cells and basophils and mediates the early humoral (sub-chronic) response on the B-side of the immune system. If an allergen binds mast cell-attached IgE, the mast cell releases mediators like histamine, and the eicosanoid leukotrienes and prostaglandins products, some of which cause the familiar symptoms of an acute allergic reaction: swelling, itching, mucous, and reddening of the skin. Later in this process eosinophils and other inflammatory cells migrate to the site of inflammation. This later phase is important in asthma, because the eosinophils may instigate a more chronic inflammation which can adversely scar lung tissue. IL-4 is at least partly responsible for recruiting eosinophils, because it induces synthesis of specific adhesion molecules on the capillary endothelium, and stimulates expression of IL-5 and eotaxin. IL-5 leads to the development of a large number of eosinophils from precursor cells in the bone marrow, and eotaxin stimulates their migration into the lung tissue.

It has been proposed that inhibition of IL-4 activity would disarm the Th2 component of the immune system. This would then allow the immune system to develop a natural tolerance towards common allergens without the full acute response to the challenge. In this way, tolerance may be induced in many patients, similar to what is sometimes achieved with hyposensitization shots for allergy patients. Thus, substances that inhibit IL-4 production and/or its binding to the IL-4 receptor, may improve the therapy of allergies and asthma.

The gene for IL-4Rα has eleven exons encoding an 825 amino acid protein and spans over 24 kb of the short arm of chromosome 16 (16p 12.1) (Pritchard et al., Genomics 10:801, 1991; GenBank Accession No. AC004525). A reference sequence for IL4R-α gene, which corresponds to the reverse complement of nucleotides 100020-71331 in the GenBank Accession No. AC004525, is shown in FIG. 1 (SEQ ID NO:1). Reference sequences for IL-4Rα mRNA (GenBank Accession No. NM_{—}000418) and the encoded IL4Rα precursor protein (GenBank Accession No. P24394) are shown in FIGS. 2 and 3, respectively (SEQ ID NOS:2 and 3). Significant features reported for the IL-4Rα precursor include: a signal peptide located between a.a. 1 and 25; an extracellular domain between a.a. 26 and 232; disulfide bonds between a.a. 34 and 44 and between a.a. 74 and 86; glycosylation sites at amino acids 53, 98, 128, 134, 176, and 209; a transmembrane region between a.a. 233 and 256; and a cytoplasmic domain between a.a. 257 and 825.

Recently, several studies have suggested that genetic polymorphisms in the IL-4-Rα gene are associated with genetic predisposition to atopy and/or elevated serum IgE. Mitsuyasu et al., reported that polymorphisms at codons 75 and 576 affect IL-4R function (Nat. Genet. 19:119-120, 1998). The IL-4Rα allele with isoleucine at amino acid position 75 (Ile75) in the extracellular domain is more responsive to IL-4 than the allele with valine at that position (Val75) and is associated with atopic asthma but not with non-atopic asthma (Mitsuyasu et al., supra). Also, the allele with arginine at position 576 (Arg576) in the cytoplasmic domain exhibits higher receptor activity than the glutamine allele (Glu576) due to reduced binding by the Arg576 allele of a negative regulatory molecule, src homology phosphatase 1 (Imani et al., J. Biol Chem. 272:7927-7931, 1997). The Arg576 allele has a higher frequency in patients with allergic inflammatory disorders, including atopy (Khurana Hershey et al., New Eng. J. Med. 337:1720-1725, 1997). In a recent study, Hershey et al., (WO 00/34789) reported that the-Arg576 allele is significantly associated with asthma. Studies showed that patients who were homozygous for this allele had about a 9-fold higher risk towards asthma and that two copies of Arg576 are associated in an increase in asthma prevalence and severity. Kanemitsu et al. recently reported that the presence of either the Ile75 or Arg576 variant is significantly associated with susceptibility for developing systemic lupus erythematosus (SLE), a Th2-dominant systemic autoinumune disorder (Arthritis Rheum. 42:1298-1300). Another variant IL-4Rα allele that is reportedly associated with atopy susceptibility has proline rather than serine at position 503 (Kruse et al., Immunol. 96:365-371, 1999). Other IL-4Rα gene polymorphisms leading to amino acid changes in the cytoplasmic domain of the protein product have been identified at codons 400 (E400A), 431 (C431R) and 786 (S786P) (Kruse et al., supra; Deichmann et al., Biochem. Biophys. Res. Commun. 231:696-697, 1997). A polymorphism of guanine or adenine at a position corresponding to nucleotide 55328 in FIG. 1 has also been reported as well as a polymorphism of cytosine or thymine at a position corresponding to nucleotide 55430 (Buetow et al., 1999, Nat Genet. 21:323-5).

Because of the potential for polymorphisms in the IL4Rα gene to affect the expression and function of the encoded protein, it would be useful to determine whether additional polymorphisms exist in the IL4Rα gene, as well as how such polymorphisms are combined in different copies of the gene. Such information would be useful for studying the biological function of IL4Rα as well as in identifying drugs targeting this protein for the treatment of disorders related to its abnormal expression or function.

### SUMMARY OF THE INVENTION

Accordingly, the inventors herein have discovered 38 novel polymorphic sites in the IL4Rα gene. These polymorphic sites (PS) correspond to the following nucleotide positions in the reverse complement of the indicated GenBank Accession Number: 32884 (PS1), 32903 (PS2), 32961 (PS3), 33135 (PS4), 35763 (PS6), 35770 (PS7), 35817 (PS8), 35905 (PS9), 35944 (PS10), 35958 (PS11), 37330 (PS12), 37473 (PS13), 37586 (PS14), 37591 (PS15), 37604 (PS16), 37644 (PS17), 37678 (PS18), 43446 (PS19), 43703 (PS20), 53008 (PS21), 53099 (PS22), 53153 (PS23), 53456 (PS25), 53507 (PS27), 53513 (PS28), 53915 (PS30), 53949 (PS32), 54237 (PS33), 54468 (PS34), 54611 (PS35), 54698 (PS36), 54700 (PS37), 54741 (PS38), 54780 (PS39), 55083 (PS40), 55142 (PS41), 55539 (PS44) and 55758 (PS45) in AC004525. The polymorphisms at these sites are adenine or guanine at PS1, cytosine or thymine at PS2, guanine or thymine at PS3, guanine or cytosine at PS4, cytosine or thymine at PS6, guanine or adenine at PS7, thymine or cytosine at PS8, cytosine or thymine at PS9, cytosine or thymine at PS10, guanine or adenine at PS11, guanine or adenine at PS12, cytosine or thymine at PS13, cytosine or thymine at PS14, guanine or adenine at PS15, adenine or thymine at PS16, cytosine or adenine at PS17, cytosine or thymine at PS18, guanine or adenine at PS19, thymine or cytosine at PS20, adenine or cytosine at PS21, cytosine or thymine at PS22, thymine or cytosine at PS23, guanine or thymine at PS25, cytosine or thymine at PS27, thymine or cytosine at PS28, cytosine or thymine at PS30, guanine or adenine at PS32, cytosine or thymine at PS33, thymine or guanine at PS34, thymine or cytosine at PS35, thymine or cytosine at PS36, thymine or cytosine at PS37, cytosine or thymine at PS38, cytosine or guanine at PS39, adenine or guanine at PS40, guanine or adenine at PS41, cytosine or thymine at PS44 and guanine or adenine at PS45. In addition, the inventors have determined the identity of the alternative nucleotides present at these sites, as well as at the previously identified sites at nucleotides 35749 (PS5), 53413 (PS24), 53505 (PS26), 53721 (PS29), 53941 (PS31), 55328 (PS42), and 55430 (PS43). It is believed that IL4Rα-encoding polynucleotides containing one or more of the novel polymorphic sites reported herein will be useful in studying the expression and biological function of IL4α, as well as in developing drugs targeting this protein. In addition, information on the combinations of polymorphisms in the IL4Rα gene may have diagnostic and forensic applications.

Thus, in one embodiment, the invention provides an isolated polynucleotide comprising a nucleotide sequence which is a polymorphic variant of a reference sequence for the IL4Rα gene or a fragment thereof. The reference sequence comprises SEQ ID NO:1 and the polymorphic variant comprises at least one polymorphism selected from the group consisting of guanine at PS1, thymine at PS2, thymine at PS3, cytosine at PS4, thymine at PS6, adenine at PS7, cytosine at PS8, thymine at PS9, thymine at PS10, adenine at PS11, adenine at PS12, thymine at PS13, thymine at PS14, adenine at PS15, thymine at PS16, adenine at PS17, thymine at PS18, adenine at PS19, cytosine at PS20, cytosine at PS21, thymine at PS22, cytosine at PS23, thymine at PS25, thymine at PS27, cytosine at PS28, thymine at PS30, adenine at PS32, thymine at PS33, guanine at PS34, cytosine at PS35, cytosine at PS36, cytosine at PS37, thymine at PS38, guanine at PS39, guanine at PS40, adenine at PS41, thymine at PS44, and adenine at PS45. In a preferred embodiment, the polymorphic variant comprises one or more additional polymorphisms selected from the group consisting of guanine at PS5, cytosine at PS24, cytosine at PS26, cytosine at PS29, guanine at PS31, adenine at PS42, and thymine at PS43. A particularly preferred polymorphic variant is a naturally-occurring isoform (also referred to herein as an “isogene”) of the IL4Rα gene. An IL4Rα isogene of the invention comprises adenine or guanine at PS1, cytosine or thymine at PS2, guanine or thymine at PS3, guanine or cytosine at PS4, cytosine or thymine at PS6, guanine or adenine at PS7, thymine or cytosine at PS8, cytosine or thymine at PS9, cytosine or thymine at PS10, guanine or adenine at PS11, guanine or adenine at PS12, cytosine or thymine at PS13, cytosine or thymine at PS14, guanine or adenine at PS15, adenine or thymine at PS16, cytosine or adenine at PS17, cytosine or thymine at PS18, guanine or adenine at PS19, thymine or cytosine at PS20, adenine or cytosine at PS21, cytosine or thymine at PS22, thymine or cytosine at PS23, guanine or thymine at PS25, cytosine or thymine at PS27, thymine or cytosine at PS28, cytosine or thymine at PS30, guanine or adenine at PS32, cytosine or thymine at PS33, thymine or guanine at PS34, thymine or cytosine at PS35, thymine or cytosine at PS36, thymine or cytosine at PS37, cytosine or thymine at PS38, cytosine or guanine at PS39, adenine or guanine at PS40, guanine or adenine at PS41, cytosine or thymine at PS44 and guanine or adenine at PS45. The invention also provides a collection of IL4Rα isogenes, referred to herein as an IL4Rα genome anthology.

An IL4Rα isogene may be defined by the combination and order of these polymorphisms in the isogene, which is referred to herein as an IL4Rα haplotype. Thus, the invention also provides data on the number of different IL4Rα haplotypes found in the above four population groups. This haplotype data is useful in methods for deriving an IL4Rα haplotype from an individual's genotype for the IL4Rα gene and for determining an association between an IL4Rα haplotype and a particular trait.

In another embodiment, the invention provides a polynucleotide comprising a polymorphic variant of a reference sequence for an IL4Rα cDNA or a fragment thereof. The reference sequence comprises SEQ ID NO:2 (FIG. 2) and the polymorphic cDNA comprises at least one polymorphism selected from the group consisting of thymine at a position corresponding to nucleotide 237, adenine at a position corresponding to nucleotide 244, cytosine at a position corresponding to nucleotide 291, thymine at a position corresponding to nucleotide 501, adenine at a position corresponding to nucleotide 554, cytosine at a position corresponding to nucleotide 939, thymine at a position corresponding to nucleotide 1242, thymine at a position corresponding to nucleotide 1293, cytosine at a position corresponding to nucleotide 1299, thymine at a position corresponding to nucleotide 1701, adenine at a position corresponding to nucleotide 1735, thymine at a position corresponding to nucleotide 2023, guanine at a position corresponding to nucleotide 2254 and cytosine at a position corresponding to nucleotide 2397. In a preferred embodiment, the polymorphic variant comprises one or more additional polymorphisms selected from the group consisting of guanine at a position corresponding to 223, cytosine at a position corresponding to nucleotide 1199, cytosine at a position corresponding to 1291, cytosine at a position corresponding to nucleotide 1507 and guanine at a position corresponding to 1737.

Polynucleotides complementary to these IL4Rα genomic and cDNA variants are also provided by the invention.

In other embodiments, the invention provides a recombinant expression vector comprising one of the polymorphic genomic variants operably linked to expression regulatory elements as well as a recombinant host cell transformed or transfected with the expression vector. The recombinant vector and host cell may be used to express IL4Rα for protein structure analysis and drug binding studies.

In yet another embodiment, the invention provides a polypeptide comprising a polymorphic variant of a reference amino acid sequence for the IL4Rα protein. The reference amino acid sequence comprises SEQ ID NO:3 (FIG. 3) and the polymorphic variant comprises at least one variant amino acid selected from the group consisting of threonine at a position corresponding to amino acid 82, histidine at a position corresponding to amino acid 185, isoleucine at a position corresponding to amino acid 579, serine at a position corresponding to amino acid 675, and alanine at a position corresponding to amino acid 752. In some embodiments, the polymorphic variant also comprises at least one variant amino acid selected from the group consisting of valine at a position corresponding to amino acid 75, alanine at a position corresponding to amino acid 400, arginine at a position correpsonding to amino acid 431, proline at a position corresponding to amino acid 503, and arginine at a position corresponding to amino acid 576. A polymorphic variant of IL4Rα is useful in studying the effect of the variation on the biological activity of IL4Rα as well as studying the binding affinity of candidate drugs targeting IL4Rα for the treatment of allergies, asthma, and other immune responses.

The present invention also provides antibodies that recognize and bind to the above polymorphic IL4Rα protein variant. Such antibodies can be utilized in a variety of diagnostic and prognostic formats and therapeutic methods.

In other embodiments, the invention provides methods, compositions, and kits for haplotyping and/or genotyping the IL4Rα gene in an individual. The methods involve identifying the nucleotide or nucleotide pair present at one or more polymorphic sites selected from PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45 in one or both copies of the IL4Rα gene from the individual. The compositions contain oligonucleotide probes and primers designed to specifically hybridize to one or more target regions containing, or that are adjacent to, a polymorphic site. The methods and compositions for establishing the genotype or haplotype of an individual at the novel polymorphic sites described herein are useful for studying the effect of the polymorphisms in the etiology of diseases affected by the expression and function of the IL4Rα protein, studying the efficacy of drugs targeting IL4Rα, predicting individual susceptibility to diseases affected by the expression and function of the IL4Rα protein and predicting individual responsiveness to drugs targeting IL4Rα.

In yet another embodiment, the invention provides a method for identifying an association between a genotype or haplotype and a trait. In preferred embodiments, the trait is susceptibility to a disease, severity of a disease, the staging of a disease or response to a drug. Such methods have applicability in developing diagnostic tests and therapeutic treatments for allergies, asthma, and other immune responses.

The present invention also provides transgenic animals comprising one of the IL4Rα genomic polymorphic variants described herein and methods for producing such animals. The transgenic animals are useful for studying expression of the IL4Rα isogenes in vivo, for in vivo screening and testing of drugs targeted against IL4Rα protein, and for testing the efficacy of therapeutic agents and compounds for allergies, asthma, and other immune responses in a biological system.

The present invention also provides a computer system for storing and displaying polymorphism data determined for the IL4Rα gene. The computer system comprises a computer processing unit; a display; and a database containing the polymorphism data. The polymorphism data includes the polymorphisms, the genotypes and the haplotypes identified for the IL4Rα gene in a reference population. In a preferred embodiment, the computer system is capable of producing a display showing IL4Rα haplotypes organized according to their evolutionary relationships.

### BRIEF DESCRIPTION OF THE DRAWINGS

FIG. 1 illustrates a reference sequence for the IL4Rα gene that is the reverse complement of part of Genbank Accession Number AC004525.1; contiguous lines; SEQ ID NO:1), with the underlines indicating the start and stop codons, shading indicating the reference coding sequence, and the bold nucleotides indicating the polymorphic sites and polymorphisms identified by Applicants in a reference population.

FIG. 2 illustrates a reference sequence for the IL4Rα coding sequence (GenBank Accession Number X52425; contiguous lines; SEQ ID NO:2), with the underlines indicating the start and stop codons, and the bold nucleotides indicating the polymorphic sites and polymorphisms identified by Applicants in a reference population.

FIG. 3 illustrates a reference sequence for the IL4Rα protein (GenBank Accession Number CAA36672; contiguous lines; SEQ ID NO:3), with the bold amino acids indicating the amino acid variations caused by the polymorphisms of FIG. 2.

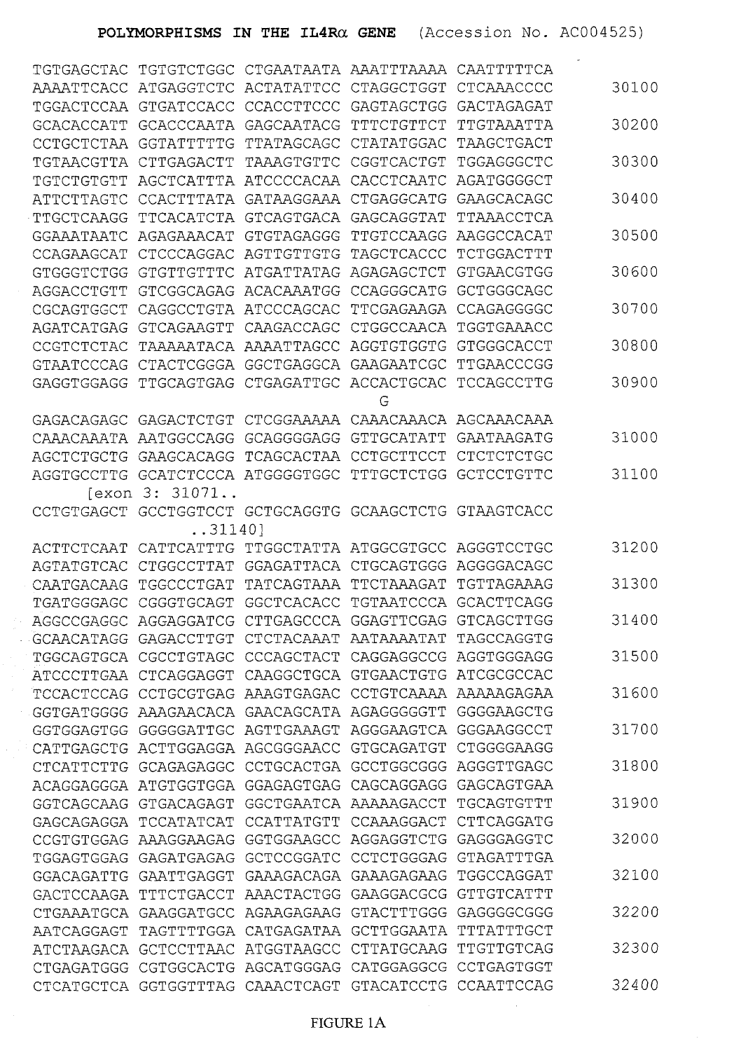

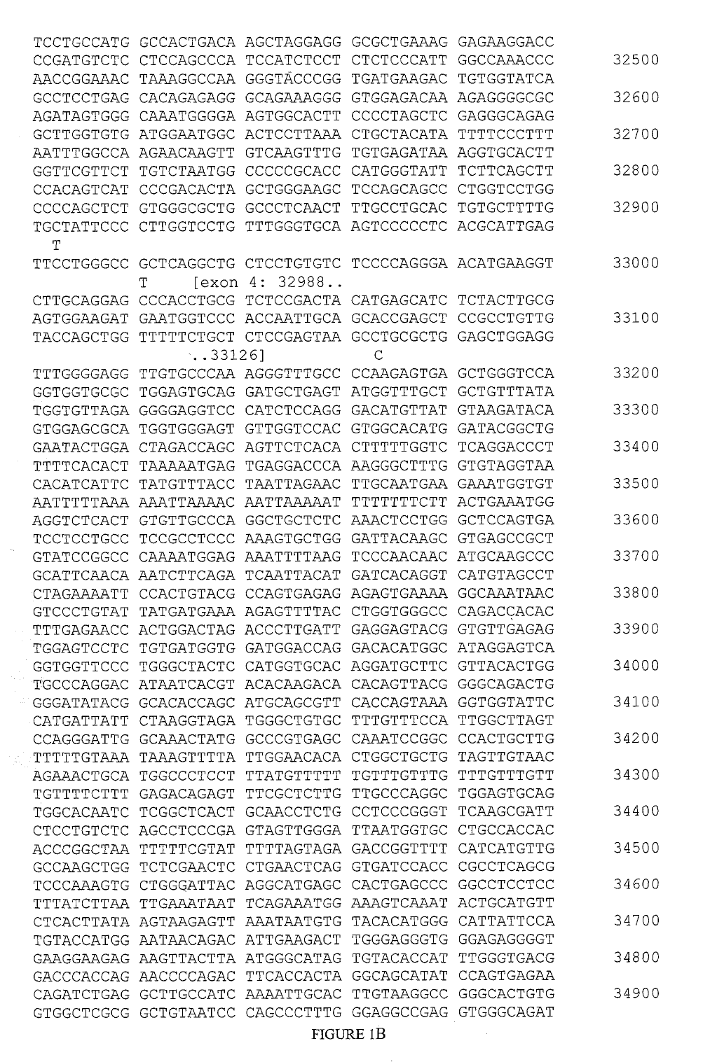

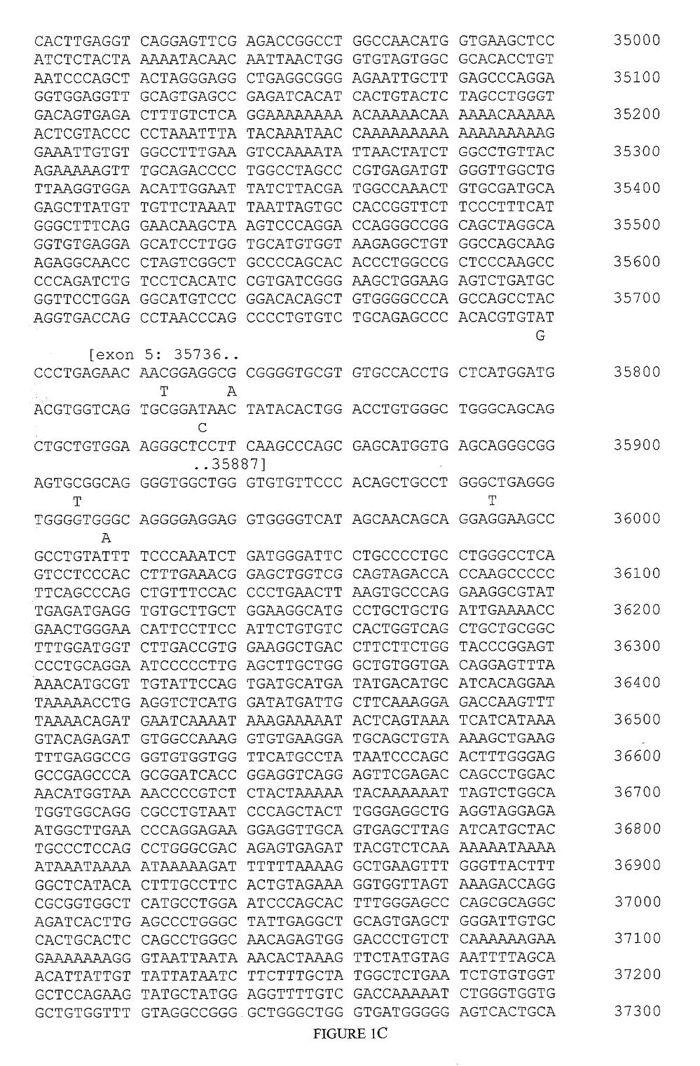

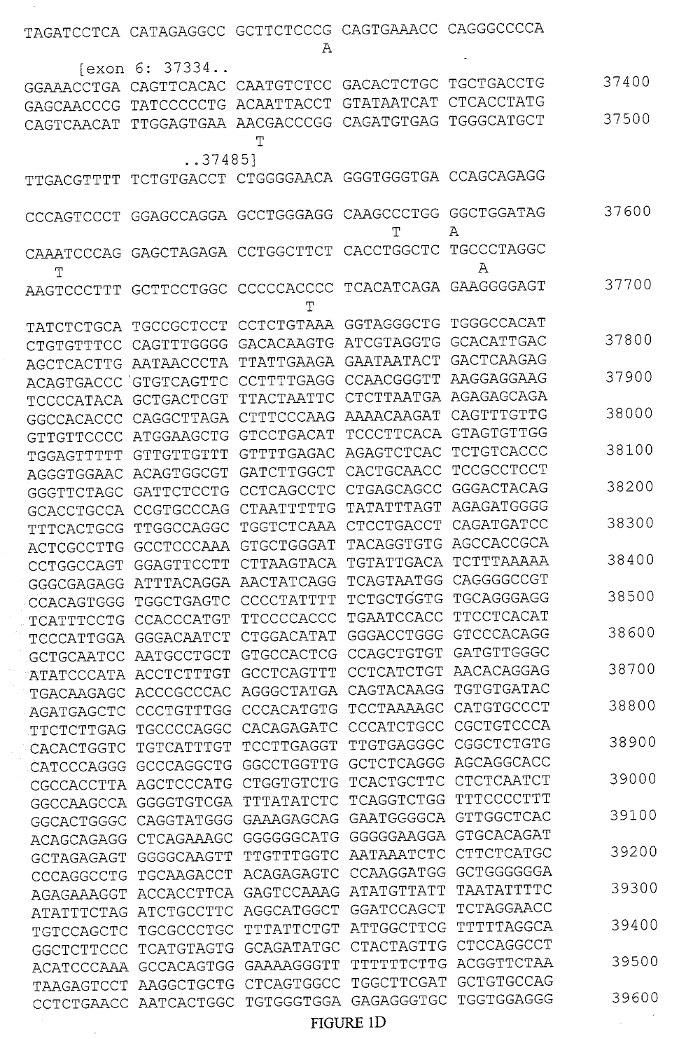

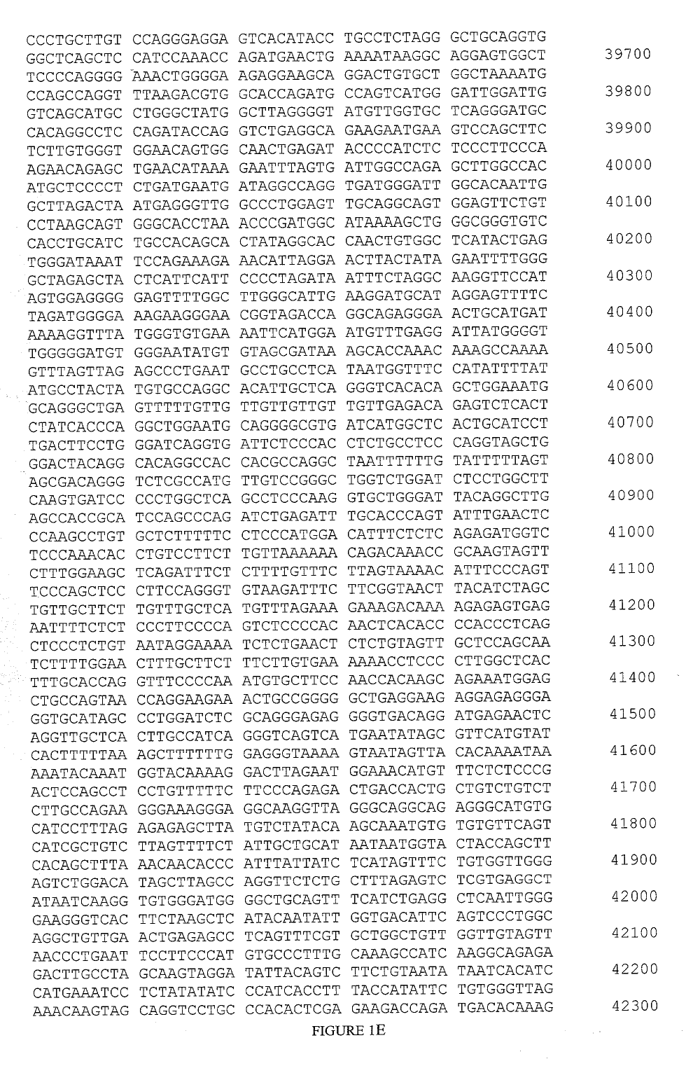

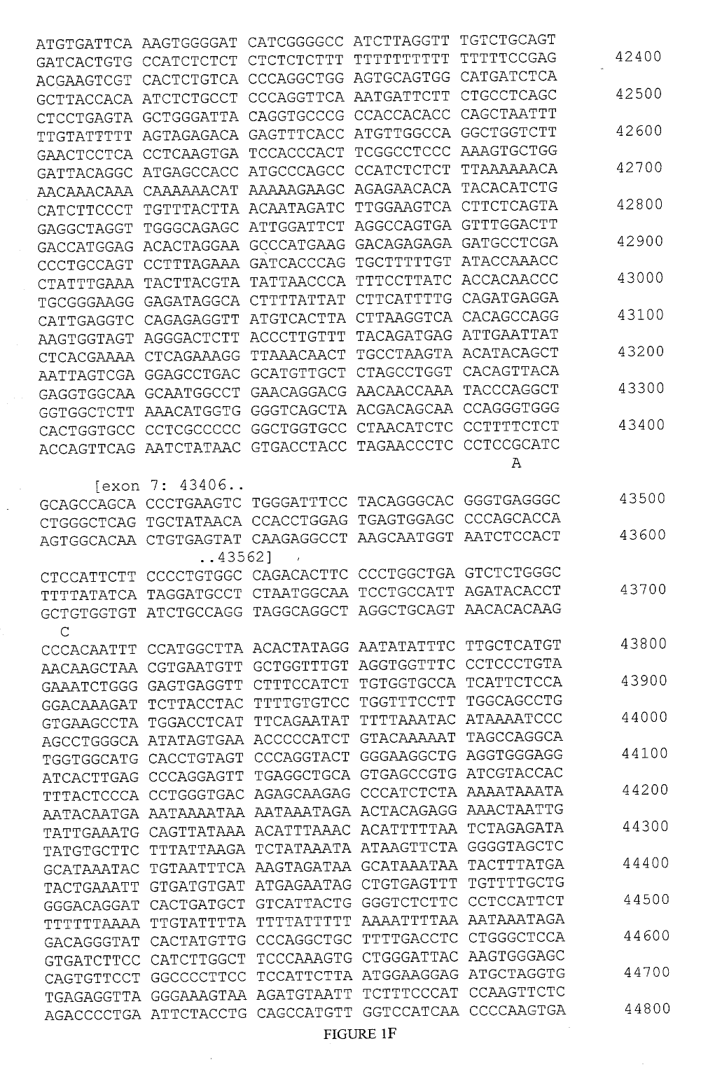

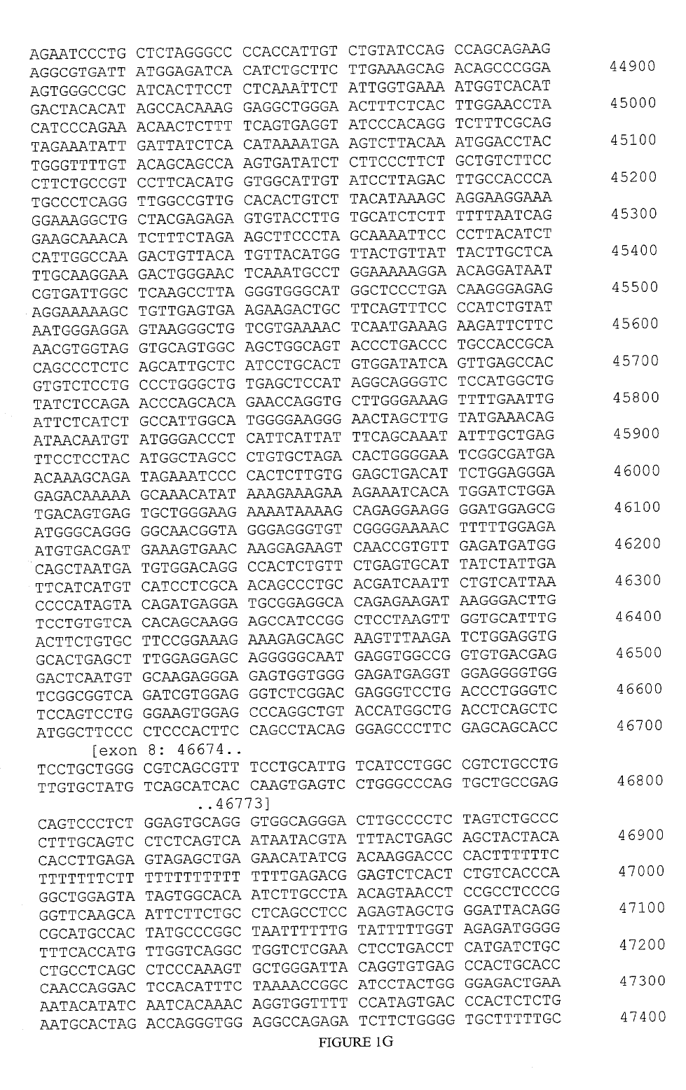

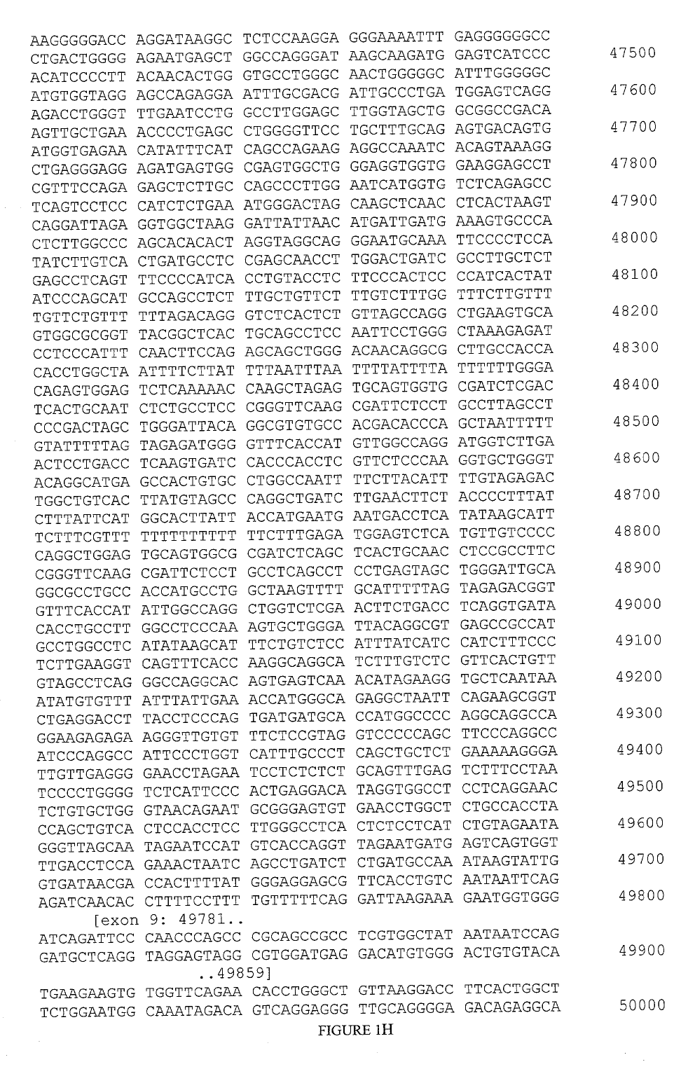

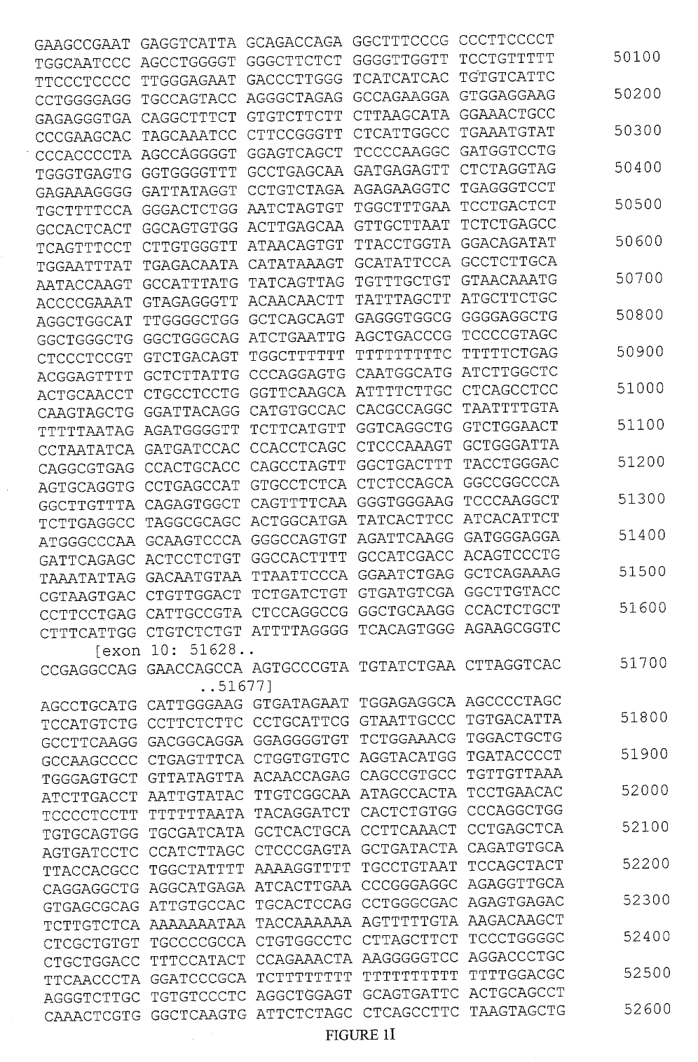

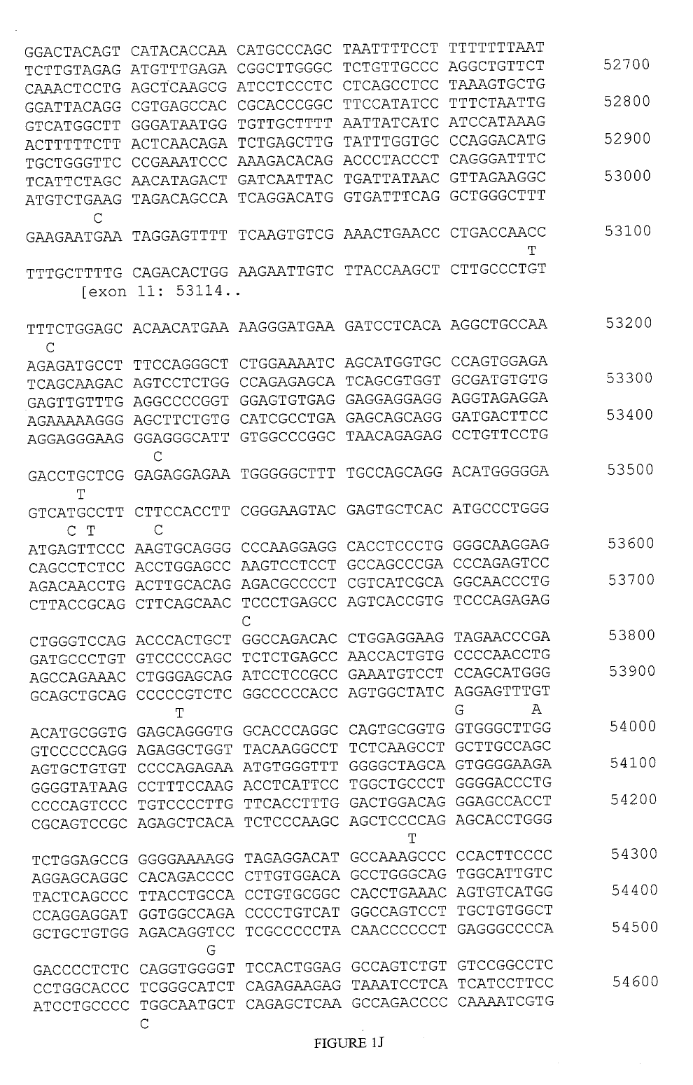

### DESCRIPTION OF THE PREFERRED EMBODIMENTS

The present invention is based on the discovery of novel variants of the IL4Rα gene. As described in more detail below, the inventors herein discovered 38 novel polymorphic sites by characterizing the IL4Rα gene found in genomic DNAs isolated from Index Repository IA that contains immortalized cell lines from one chimpanzee and 93 human individuals and Index Repository IB that contains 70 human individuals. Theses two repositories contain 51 individuals in common.

The human individuals in Index Repository IA included a reference population of 79 unrelated individuals self-identified as belonging to one of four major population groups: Caucasian (22 individuals), African descent (20 individuals) Asian (20 individuals) Hispanic/Latino (17 individuals). To the extent possible, the members of this reference population were organized into population subgroups by the self-identified ethnogeographic origin of their four grandparents as shown in Table 1 below. In addition, Index Repository IA contains three unrelated indigenous American Indians (one from each of North, Central, and South America), one three-generation Caucasian family (From the CEPH Utah cohort) and one two-generation African-American family.

TABLE 1
Population Groups in Index Repository IA

| | | No. of |
|---|---|---|
| Population Group | Population Subgroup | Individuals |
| African descent | | 20 |
| | Sierra Leone | 1 |
| Asian | | 20 |
| | Burma | 1 |
| | China | 3 |
| | Japan | 6 |
| | Korea | 1 |
| | Philippines | 5 |
| | Vietnam | 4 |
| Caucasian | | 22 |
| | British Isles | 3 |
| | British Isles/Central | 4 |
| | British Isles/Eastern | 1 |
| | Central/Eastern | 1 |
| | Eastern | 3 |
| | Central/Mediterranean | 1 |
| | Mediterranean | 2 |
| | Scandinavian | 2 |
| Hispanic/Latino | | 17 |
| | Caribbean | 7 |
| | Caribbean (Spanish Descent) | 2 |
| | Central American (Spanish Descent) | 1 |
| | Mexican American | 4 |
| | South American (Spanish Descent) | 3 |

Index Repository IB contains a reference population of 70 human individuals comprised of 4 three-generation families (from the CEPH Utah cohort) as well as unrelated African-American, Asian, and Caucasian individuals. A total of 38 individuals in this reference population are unrelated.

Using the IL4Rα genotypes identified in the Index Repositories and the methodology described in the Examples below, the inventors herein also determined the haplotypes found on each chromosome for most human members of this repository. The IL4α genotypes and haplotypes found in the Index Repositories include those shown in Tables 4 and 5, respectively. The polymorphism and haplotype data disclosed herein are useful for studying population diversity, anthropological lineage, the significance of diversity and lineage at the phenotypic level, paternity testing, forensic applications, and for identifying associations between the IL4Rα genetic variation and a trait such as level of drug response or susceptibility to disease.

In the context of this disclosure, the following terms shall be defined as follows unless otherwise indicated:

Allele—A particular form of a genetic locus, distinguished from other forms by its particular nucleotide sequence.

Candidate Gene—A gene which is hypothesized to be responsible for a disease, condition, or the response to a treatment, or to be correlated with one of these.

Gene—A segment of DNA that contains all the information for the regulated biosynthesis of an RNA product, including promoters, exons, introns, and other untranslated regions that control expression.

Genotype—An unphased 5′ to 3′ sequence of nucleotide pair(s) found at one or more polymorphic sites in a locus on a pair of homologous chromosomes in an individual. As used herein, genotype includes a full-genotype and/or a sub-genotype as described below.

Full-genotype—The unphased 5′ to 3′ sequence of nucleotide pairs found at all known polymorphic sites in a locus on a pair of homologous chromosomes in a single individual.

Sub-genotype—The unphased 5′ to 3′ sequence of nucleotides seen at a subset of the known polymorphic sites in a locus on a pair of homologous chromosomes in a single individual.

Genotyping—A process for determining a genotype of an individual.

Haplotype—A 5′ to 3′ sequence of nucleotides found at one or more polymorphic sites in a locus on a single chromosome from a single individual. As used herein, haplotype includes a fall-haplotype and/or a sub-haplotype as described below.

Full-haplotype—The 5′ to 3′ sequence of nucleotides found at all known polymorphic sites in a locus on a single chromosome from a single individual.

Sub-haplotype—The 5′ to 3′ sequence of nucleotides seen at a subset of the known polymorphic sites in a locus on a single chromosome from a single individual.

Haplotype pair—The two haplotypes found for a locus in a single individual.

Haplotyping—A process for determining one or more haplotypes in an individual and includes use of family pedigrees, molecular techniques and/or statistical inference.

Haplotype data—Information concerning one or more of the following for a specific gene: a listing of the haplotype pairs in each individual in a population; a listing of the different haplotypes in a population; frequency of each haplotype in that or other populations, and any known associations between one or more haplotypes and a trait.

Isoform—A particular form of a gene, mRNA, cDNA or the protein encoded thereby, distinguished from other forms by its particular sequence and/or structure.

Isogene—One of the isoforms of a gene found in a population. An isogene contains all of the polymorphisms present in the particular isoform of the gene.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Locus—A location on a chromosome or DNA molecule corresponding to a gene or a physical or phenotypic feature.

Naturally-occurring—A term used to designate that the object it is applied to, e.g., naturally-occurring polynucleotide or polypeptide, can be isolated from a source in nature and which has not been intentionally modified by man.

Nucleotide pair—The nucleotides found at a polymorphic site on the two copies of a chromosome from an individual.

Phased—As applied to a sequence of nucleotide pairs for two or more polymorphic sites in a locus, phased means the combination of nucleotides present at those polymorphic sites on a single copy of the locus is known.

Polymorphic site (PS)—A position within a locus at which at least two alternative sequences are found in a population, the most frequent of which has a frequency of no more than 99%.

Polymorphic variant—A gene, mRNA, cDNA, polypeptide or peptide whose nucleotide or amino acid sequence varies from a reference sequence due to the presence of a polymorphism in the gene.

Polymorphism—The sequence variation observed in an individual at a polymorphic site. Polymorphisms include nucleotide substitutions, insertions, deletions and microsatellites and may, but need not, result in detectable differences in gene expression or protein function.

Polymorphism data—Information concerning one or more of the following for a specific gene: location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene.

Polymorphism Database—A collection of polymorphism data arranged in a systematic or methodical way and capable of being individually accessed by electronic or other means.

Polynucleotide—A nucleic acid molecule comprised of single-stranded RNA or DNA or comprised of complementary, double-stranded DNA.

Population Group—A group of individuals sharing a common ethnogeographic origin.

Reference Population—A group of subjects or individuals who are predicted to be representative of the genetic variation found in the general population. Typically, the reference population represents the genetic variation in the population at a certainty level of at least 85%, preferably at least 90%, more preferably at least 95% and even more preferably at least 99%.

Single Nucleotide Polymorphism (SNP)—Typically, the specific pair of nucleotides observed at a single polymorphic site. In rare cases, three or four nucleotides may be found.

Subject—A human individual whose genotypes or haplotypes or response to treatment or disease state are to be determined.

Treatment—A stimulus administered internally or externally to a subject.

Unphased—As applied to a sequence of nucleotide pairs for two or more polymorphic sites in a locus, unphased means the combination of nucleotides present at those polymorphic sites on a single copy of the locus is not known.

The inventors herein have discovered 38 novel polymorphic sites, and confirmed the existence of 7 other sites, in the IL4Rα gene. The polymorphic sites identified by the inventors are referred to as PS1-45 to designate the order in which they are located in the gene (see Table 3 below), with the novel polymorphic site referred to as PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45 and the previously reported poly sites referred to as PS5, PS24, PS26, PS29, PS31, PS42, and PS43.

Thus, in one embodiment, the invention provides an isolated polynucleotide comprising a polymorphic variant of the IL4Rα gene or a fragment of the gene which contains at least one of the novel polymorphic sites described herein. The nucleotide sequence of a variant IL4Rα gene is identical to the reference genomic sequence for those portions of the gene examined, as described in the Examples below, except that it comprises a different nucleotide at one or more of the novel polymorphic sites PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45, and may also comprise one or more additional polymorphisms selected from the group consisting of PS5, PS24, PS26, PS29, PS31, PS42, and PS43. Similarly, the nucleotide sequence of a variant fragment of the IL4Rα gene is identical to the corresponding portion of the reference sequence except for having a different nucleotide at one or more of the novel polymorphic sites described herein. Thus, the invention specifically does not include polynucleotides comprising a nucleotide sequence identical to the reference sequence (or other reported IL4Rα sequences) or to portions of the reference sequence (or other reported IL4Rα sequences), except for genotyping oligonucleotides as described below.

The location of a polymorphism in a variant gene or fragment is identified by aligning its sequence against SEQ ED NO:1. The polymorphism is selected from the group consisting of guanine at PS1, thymine at PS2, thymine at PS3, cytosine at PS4, thymine at PS6, adenine at PS7, cytosine at PS8, thymine at PS9, thymine at PS10, adenine at PS11, adenine at PS12, thymine at PS13, thymine at PS14, adenine at PS15, thymine at PS16, adenine at PS17, thymine at PS18, adenine at PS19, cytosine at PS20, cytosine at PS21, thymine at PS22, cytosine at PS23, thymine at PS25, thymine at PS27, cytosine at PS28, thymine at PS30, adenine at PS32, thymine at PS33, guanine at PS34, cytosine at PS35, cytosine at PS36, cytosine at PS37, thymine at PS38, guanine at PS39, guanine at PS40, adenine at PS41, thymine at PS44, and adenine at PS45. In a preferred embodiment, the polymorphic variant comprises a naturally-occurring isogene of the IL4Rα gene which is defined by any one of haplotypes 1-53 shown in Table 5 below.

Polymorphic variants of the invention may be prepared by isolating a clone containing the IL4Rα gene from a human genomic library. The clone may be sequenced to determine the identity of the nucleotides at the polymorphic sites described herein. Any particular variant claimed herein could be prepared from this clone by performing in vitro mutagenesis using procedures well-known in the art.

IL4Rα isogenes may be isolated using any method that allows separation of the two “copies” of the IL4Rα gene present in an individual, which, as readily understood by the skilled artisan, may be the same allele or different alleles. Separation methods include targeted in vivo cloning (TIVC) in yeast as described in WO 98/01573, U.S. Pat. No. 5,866,404, and copending U.S. application Serial No. 08/987,966. Another method, which is described in copending U.S. application Ser. No. 08/987,966, uses an allele specific oligonucleotide in combination with primer extension and exonuclease degradation to generate hemizygous DNA targets. Yet other methods are single molecule dilution (SMD) as described in Ruaño et al., Proc. Natl. Acad. Sci. 87:6296-6300, 1990; and allele specific PCR (Ruaño et al., 17 Nucleic Acids. Res. 8392, 1989; Ruaño et al., 19 Nucleic Acids Res. 6877-6882, 1991; Michalatos-Beloin et al., 24 Nucleic Acids Res. 4841-4843, 1996).

The invention also provides IL4Rα genome anthologies, which are collections of IL4Rα isogenes found in a given population. The population may be any group of at least two individuals, including but not limited to a reference population, a population group, a family population, a clinical population, and a same sex population. An IL4Rα genome anthology may comprise individual IL4Rα isogenes stored in separate containers such as microtest tubes, separate wells of a microtitre plate and the like. Alternatively, two or more groups of the IL4Rα isogenes in the anthology may be stored in separate containers. Individual isogenes or groups of isogenes in a genome anthology may be stored in any convenient and stable form, including but not limited to in buffered solutions, as DNA precipitates, freeze-dried preparations and the like. A preferred IL4Rα genome anthology of the invention comprises a set of isogenes defined by the haplotypes shown in Table 5 below.

An isolated polynucleotide containing a polymorphic variant nucleotide sequence of the invention may be operably linked to one or more expression regulatory elements in a recombinant expression vector capable of being propagated and expressing the encoded IL4Rα protein in a prokaryotic or a eukaryotic host cell. Examples of expression regulatory elements which may be used include, but are not limited to, the lac system, operator and promoter regions of phage lambda, yeast promoters, and promoters derived from vaccinia virus, adenovirus, retroviruses, or SV40. Other regulatory elements include, but are not limited to, appropriate leader sequences, termination codons, polyadenylation signals, and other sequences required for the appropriate transcription and subsequent translation of the nucleic acid sequence in a given host cell. Of course, the correct combinations of expression regulatory elements will depend on the host system used. In addition, it is understood that the expression vector contains any additional elements necessary for its transfer to and subsequent replication in the host cell. Examples of such elements include, but are not limited to, origins of replication and selectable markers. Such expression vectors are commercially available or are readily constructed using methods known to those in the art (e.g., F. Ausubel et al., 1987, in “Current Protocols in Molecular Biology”, John Wiley and Sons, New York, N.Y.). Host cells which may be used to express the variant IL4Rα sequences of the invention include, but are not limited to, eukaryotic and mammalian cells, such as animal, plant, insect and yeast cells, and prokaryotic cells, such as E. coli, or algal cells as known in the art. The recombinant expression vector may be introduced into the host cell using any method known to those in the art including, but not limited to, microinjection, electroporation, particle bombardment, transduction, and transfection using DEAE-dextran, lipofection, or calcium phosphate (see e.g., Sambrook et al. (1989) in “Molecular Cloning. A Laboratory Manual”, Cold Spring Harbor Press, Plainview, N.Y.). In a preferred aspect, eukaryotic expression vectors that function in eukaryotic cells, and preferably mammalian cells, are used. Non-limiting examples of such vectors include vaccinia virus vectors, adenovirus vectors, herpes virus vectors, and baculovirus transfer vectors. Preferred eukaryotic cell lines include COS cells, CHO cells, HeLa cells, NIH/3T3 cells, and embryonic stem cells (Thomson, J. A. et al., 1998 Science 282:1145-1147). Particularly preferred host cells are mammalian cells.

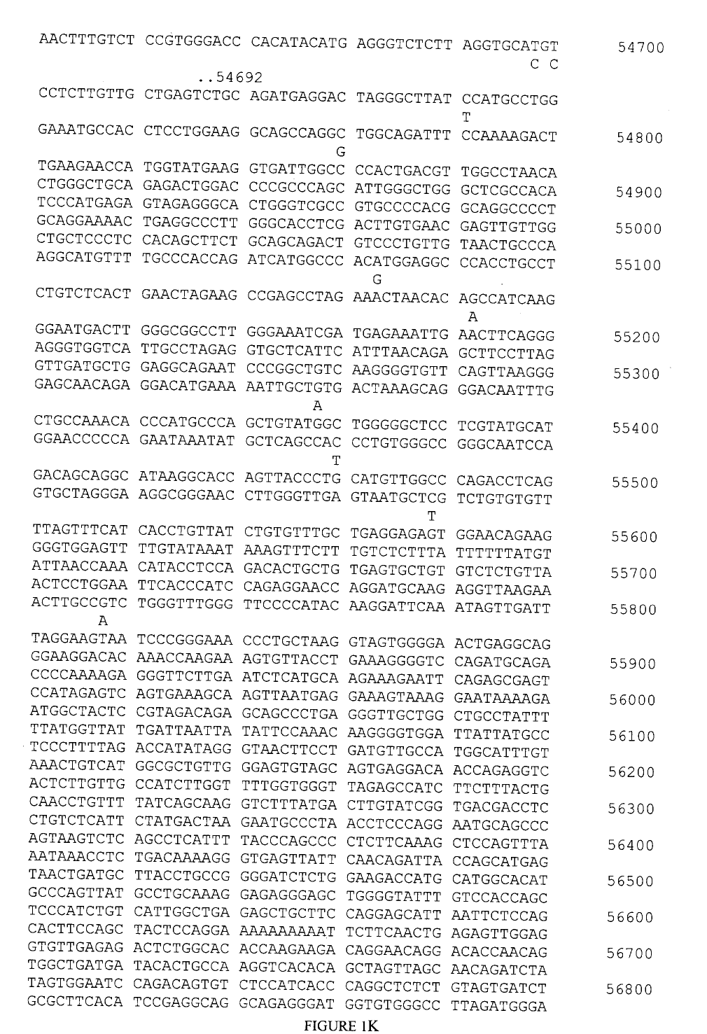

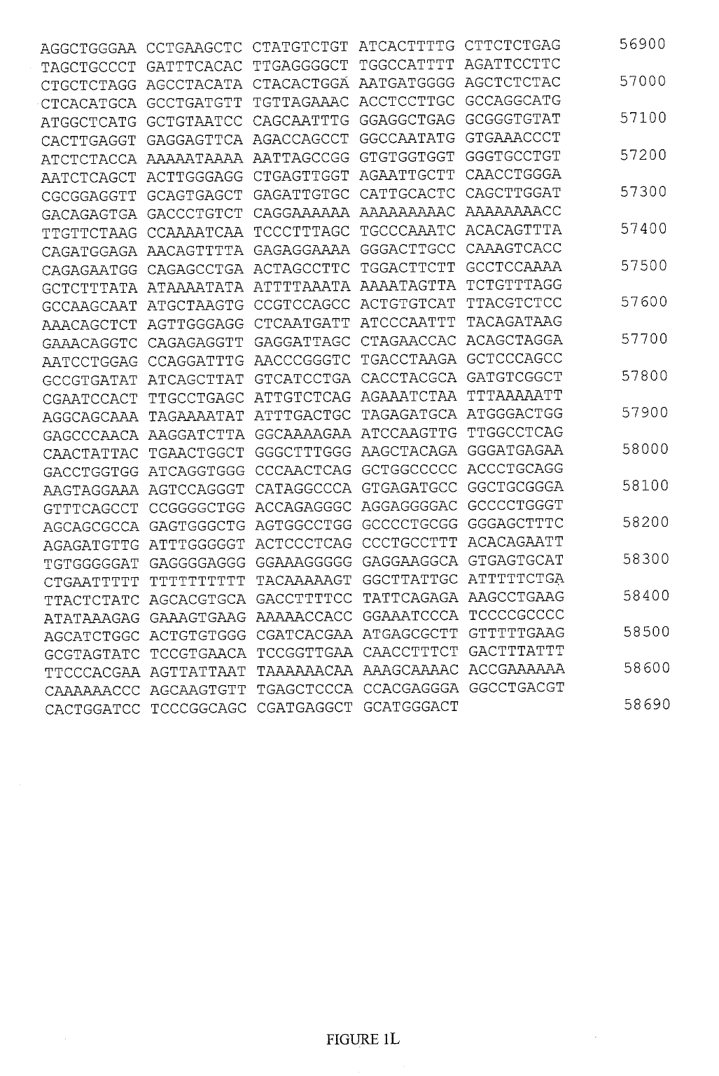

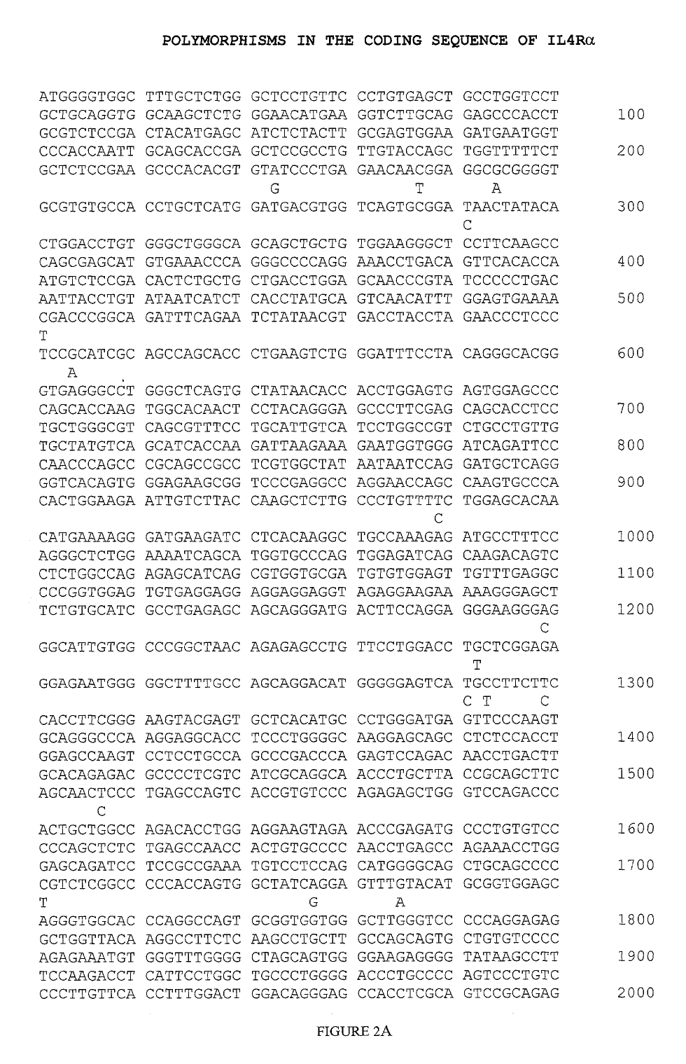

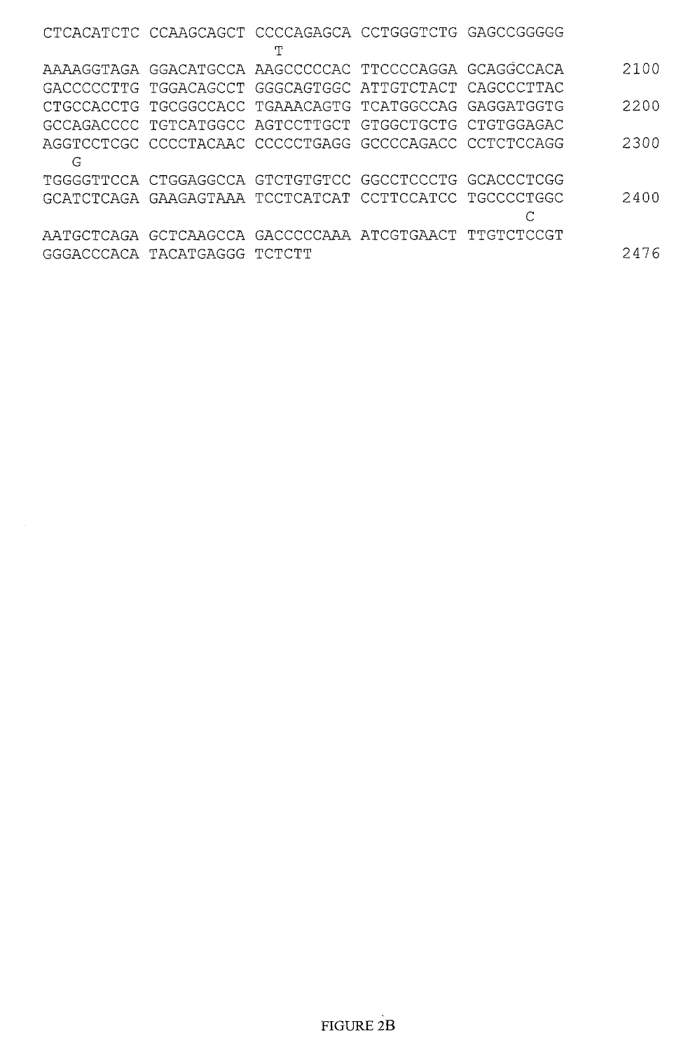

As will be readily recognized by the skilled artisan, expression of polymorphic variants of the IL4Rα gene will produce IL4Rα mRNAs varying from each other at any polymorphic site retained in the spliced and processed mRNA molecules. These mRNAs can be used for the preparation of an IL4Rα cDNA comprising a nucleotide sequence which is a polymorphic variant of the IL4Rα reference coding sequence shown in FIG. 2. Thus, the invention also provides IL4Rα mRNAs and corresponding cDNAs which comprise a nucleotide sequence that is identical to SEQ ID NO:2 (FIG. 2), or its corresponding RNA sequence, except for having one or more polymorphisms selected from the group consisting of thymine at a position corresponding to nucleotide 237, adenine at a position corresponding to nucleotide 244, cytosine at a position corresponding to nucleotide 291, thymine at a position corresponding to nucleotide 501, adenine at a position corresponding to nucleotide 554, cytosine at a position corresponding to nucleotide 939, thymine at a position corresponding to nucleotide 1242, thymine at a position corresponding to nucleotide 1293, cytosine at a position corresponding to nucleotide 1299, thymine at a position corresponding to nucleotide 1701, adenine at a position corresponding to nucleotide 1735, thymine at a position corresponding to nucleotide 2023, guanine at a position corresponding to nucleotide 2254 and cytosine at a position corresponding to nucleotide 2397, and may also comprise one or more additional polymorphisms selected from the group consisting of guanine at a position corresponding to 223, cytosine at a position corresponding to nucleotide 1199, cytosine at a position corresponding to 1291, cytosine at a position corresponding to nucleotide 1507 and guanine at a position corresponding to 1737. Fragments of these variant mRNAs and cDNAs are included in the scope of the invention, provided they contain the novel polymorphisms described herein. The invention specifically excludes polynucleotides identical to previously identified and characterized IL4Rα cDNAs and fragments thereof. Polynucleotides comprising a variant RNA or DNA sequence may be isolated from a biological sample using well-known molecular biological procedures or may be chemically synthesized.

Genomic and cDNA fragments of the invention comprise at least one novel polymorphic site identified herein and have a length of at least 10 nucleotides and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

In describing the polymorphic sites identified herein, reference is made to the sense strand of the gene for convenience. However, as recognized by the skilled artisan, nucleic acid molecules containing the IL4Rα gene may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides which are complementary to the sense strand of the IL4Rα genomic variants described herein.

Polynucleotides comprising a polymorphic gene variant or fragment may be useful for therapeutic purposes. For example, where a patient could benefit from expression, or increased expression, of a particular IL4Rα protein isoform, an expression vector encoding the isoform may be administered to the patient. The patient may be one who lacks the IL4Rα isogene encoding that isoform or may already have at least one copy of that isogene.

In other situations, it may be desirable to decrease or block expression of a particular IL4Rα isogene. Expression of an IL4Rα isogene may be turned off by transforming a targeted organ, tissue or cell population with an expression vector that expresses high levels of untranslatable mRNA for the isogene. Alternatively, oligonucleotides directed against the regulatory regions (e.g., promoter, introns, enhancers, 3′ untranslated region) of the isogene may block transcription. Oligonucleotides targeting the transcription initiation site, e.g., between positions −10 and +10 from the start site are preferred. Similarly, inhibition of transcription can be achieved using oligonucleotides that base-pair with region(s) of the isogene DNA to form triplex DNA (see e.g., Gee et al. in Huber, B. E. and B. I. Carr, Molecular and Immunologic Approaches, Futura Publishing Co., Mt. Kisco, N.Y., 1994). Antisense oligonucleotides may also be designed to block translation of IL4Rα mRNA transcribed from a particular isogene. It is also contemplated that ribozymes may be designed that can catalyze the specific cleavage of IL4Rα mRNA transcribed from a particular isogene.

The oligonucleotides may be delivered to a target cell or tissue by expression from a vector introduced into the cell or tissue in vivo or ex vivo. Alternatively, the oligonucleotides may be formulated as a pharmaceutical composition for administration to the patient. Oligoribonucleotides and/or oligodeoxynucleotides intended for use as antisense oligonucleotides may be modified to increase stability and half-life. Possible modifications include, but are not limited to phosphorothioate or 2′ O-methyl linkages, and the inclusion of nontraditional bases such as inosine and queosine, as well as acetyl-, methyl-, thio-, and similarly modified forms of adenine, cytosine, guanine, thymine, and uracil which are not as easily recognized by endogenous nucleases.

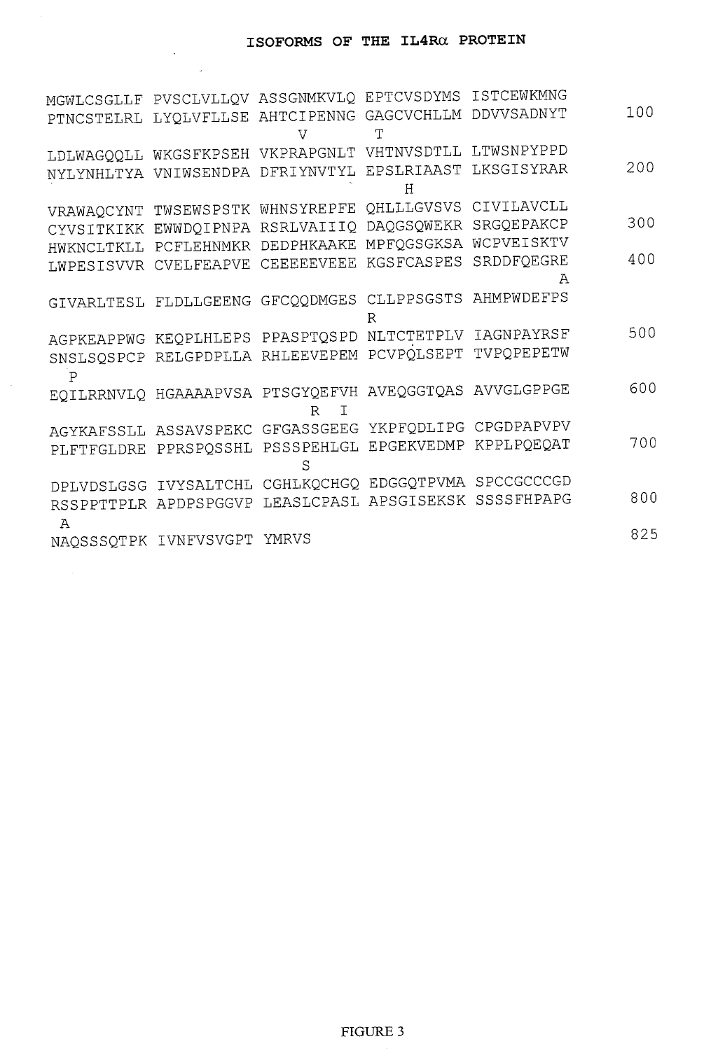

The invention also provides an isolated polypeptide comprising a polymorphic variant of the reference IL4Rα amino acid sequence shown in 3. The location of a variant amino acid in an IL4Rα polypeptide or fragment of the invention is identified by aligning its sequence against FIG. 3. An IL4Rα protein variant of the invention comprises an amino acid sequence identical to SEQ ID NO: 3 except for having one or more variant amino acids selected from the group consisting of threonine at a position corresponding to amino acid 82, histidine at a position corresponding to amino acid 185, isoleucine at a position corresponding to amino acid 579, serine at a position corresponding to amino acid 675, and alanine at a position corresponding to amino acid 752, and may also comprise one or more additional variant amino acids selected from the group consisting of valine at a position corresponding to amino acid 75, alanine at a position corresponding to amino acid 400, arginine at a position correpsonding to amino acid 431, proline at a position corresponding to amino acid 503, and arginine at a position corresponding to amino acid 576. The invention specifically excludes amino acid sequences identical to those previously identified for IL4Rα including SEQ ID NO: 3, and previously described fragments thereof. IL4Rα protein variants included within the invention comprise all amino acid sequences based on SEQ ID NO: 3 and having the combination of amino acid variations described in Table 2 below. In preferred embodiments, an IL4Rα protein variant of the invention is encoded by an isogene defined by one of the observed haplotypes shown in Table 5.

TABLE 2
Novel Polymorphic Variant of IL4R^{α}

| Polymorphic | | | | | | | | | | |
|---|---|---|---|---|---|---|---|---|---|---|
| Variant | Amino Acid Position and Identities | | | | | | | | | |
| Number | 75 | 82 | 185 | 400 | 431 | 503 | 576 | 579 | 675 | 752 |
| 1 | I | A | R | E | C | S | Q | V | P | A |
| 2 | I | A | R | E | C | S | Q | V | S | S |
| 3 | I | A | R | E | C | S | Q | V | S | A |
| 4 | I | A | R | E | C | S | Q | I | P | S |
| 5 | I | A | R | E | C | S | Q | I | P | A |
| 6 | I | A | R | E | C | S | Q | I | S | S |
| 7 | I | A | R | E | C | S | Q | I | S | A |
| 8 | I | A | R | E | C | S | R | V | P | A |
| 9 | I | A | R | E | C | S | R | V | S | S |
| 10 | I | A | R | E | C | S | R | V | S | A |
| 11 | I | A | R | E | C | S | R | I | P | S |
| 12 | I | A | R | E | C | S | R | I | P | A |
| 13 | I | A | R | E | C | S | R | I | S | S |
| 14 | I | A | R | E | C | S | R | I | S | A |
| 15 | I | A | R | E | C | P | Q | V | P | A |
| 16 | I | A | R | E | C | P | Q | V | S | S |
| 17 | I | A | R | E | C | P | Q | V | S | A |
| 18 | I | A | R | E | C | P | Q | I | P | S |
| 19 | I | A | R | E | C | P | Q | I | P | A |
| 20 | I | A | R | E | C | P | Q | I | S | S |
| 21 | I | A | R | E | C | P | Q | I | S | A |
| 22 | I | A | R | E | C | P | R | V | P | A |
| 23 | I | A | R | E | C | P | R | V | S | S |
| 24 | I | A | R | E | C | P | R | V | S | A |
| 25 | I | A | R | E | C | P | R | I | P | S |
| 26 | I | A | R | E | C | P | R | I | P | A |
| 27 | I | A | R | E | C | P | R | I | S | S |
| 28 | I | A | R | E | C | P | R | I | S | A |
| 29 | I | A | R | E | R | S | Q | V | P | A |
| 30 | I | A | R | E | R | S | Q | V | S | S |
| 31 | I | A | R | E | R | S | Q | V | S | A |
| 32 | I | A | R | E | R | S | Q | I | P | S |
| 33 | I | A | R | E | R | S | Q | I | P | A |
| 34 | I | A | R | E | R | S | Q | I | S | S |
| 35 | I | A | R | E | R | S | Q | I | S | A |
| 36 | I | A | R | E | R | S | R | V | P | A |
| 37 | I | A | R | E | R | S | R | V | S | S |
| 38 | I | A | R | E | R | S | R | V | S | A |
| 39 | I | A | R | E | R | S | R | I | P | S |
| 40 | I | A | R | E | R | S | R | I | P | A |
| 41 | I | A | R | E | R | S | R | I | S | S |
| 42 | I | A | R | E | R | S | R | I | S | A |
| 43 | I | A | R | E | R | P | Q | V | P | A |
| 44 | I | A | R | E | R | P | Q | V | S | S |
| 45 | I | A | R | E | R | P | Q | V | S | A |
| 46 | I | A | R | E | R | P | Q | I | P | S |
| 47 | I | A | R | E | R | P | Q | I | P | A |
| 48 | I | A | R | E | R | P | Q | I | S | S |
| 49 | I | A | R | E | R | P | Q | I | S | A |
| 50 | I | A | R | E | R | P | R | V | P | A |
| 51 | I | A | R | E | R | P | R | V | S | S |
| 52 | I | A | R | E | R | P | R | V | S | A |
| 53 | 1 | A | R | E | R | P | R | I | P | S |
| 54 | I | A | R | E | R | P | R | I | P | A |
| 55 | I | A | R | E | R | P | R | I | S | S |
| 56 | I | A | R | E | R | P | R | I | S | A |
| 57 | I | A | R | A | C | S | Q | V | P | A |
| 58 | I | A | R | A | C | S | Q | V | S | S |
| 59 | I | A | R | A | C | S | Q | V | S | A |
| 60 | I | A | R | A | C | S | Q | I | P | S |
| 61 | I | A | R | A | C | S | Q | I | P | A |
| 62 | I | A | R | A | C | S | Q | I | S | S |
| 63 | I | A | R | A | C | S | Q | I | S | A |
| 64 | I | A | R | A | C | S | R | V | P | A |
| 65 | I | A | R | A | C | S | R | V | S | S |
| 66 | I | A | R | A | C | S | R | V | S | A |
| 67 | I | A | R | A | C | S | R | I | P | S |
| 68 | I | A | R | A | C | S | R | I | P | A |
| 69 | I | A | R | A | C | S | R | I | S | S |
| 70 | I | A | R | A | C | S | R | I | S | A |
| 71 | I | A | R | A | C | P | Q | V | P | A |
| 72 | I | A | R | A | C | P | Q | V | S | S |
| 73 | I | A | R | A | C | P | Q | V | S | A |
| 74 | I | A | R | A | C | P | Q | I | P | S |
| 75 | I | A | R | A | C | P | Q | I | P | A |
| 76 | I | A | R | A | C | P | Q | I | S | S |
| 77 | I | A | R | A | C | P | Q | I | S | A |
| 78 | I | A | R | A | C | P | R | V | P | A |
| 79 | I | A | R | A | C | P | R | V | S | S |
| 80 | I | A | R | A | C | P | R | V | S | A |
| 81 | I | A | R | A | C | P | R | I | P | S |
| 82 | I | A | R | A | C | P | R | I | P | A |
| 83 | I | A | R | A | C | P | R | I | S | S |
| 84 | I | A | R | A | C | P | R | I | S | A |
| 85 | I | A | R | A | R | S | Q | V | P | A |
| 86 | I | A | R | A | R | S | Q | V | S | S |
| 87 | I | A | R | A | R | S | Q | V | S | A |
| 88 | I | A | R | A | R | S | Q | I | P | S |
| 89 | I | A | R | A | R | S | Q | I | P | A |
| 90 | I | A | R | A | R | S | Q | I | S | S |
| 91 | I | A | R | A | R | S | Q | I | S | A |
| 92 | I | A | R | A | R | S | R | V | P | A |
| 93 | I | A | R | A | R | S | R | V | S | S |
| 94 | i | A | R | A | R | S | R | V | S | A |
| 95 | i | A | R | A | R | S | R | I | P | S |
| 96 | I | A | R | A | R | S | R | I | P | A |
| 97 | I | A | R | A | R | S | R | I | S | S |
| 98 | I | A | R | A | R | S | R | I | S | A |
| 99 | I | A | R | A | R | P | Q | V | P | A |
| 100 | I | A | R | A | R | P | Q | V | S | S |
| 101 | I | A | R | A | R | P | Q | V | S | A |
| 102 | I | A | R | A | R | P | Q | I | P | S |
| 103 | I | A | R | A | R | P | Q | I | P | A |
| 104 | I | A | R | A | R | P | Q | I | S | S |
| 105 | I | A | R | A | R | P | Q | I | S | A |
| 106 | I | A | R | A | R | P | R | V | P | A |
| 107 | I | A | R | A | R | P | R | V | S | S |
| 108 | I | A | R | A | R | P | R | V | S | A |
| 109 | I | A | R | A | R | P | R | I | P | S |
| 110 | I | A | R | A | R | P | R | I | P | A |
| 111 | I | A | R | A | R | P | R | I | S | S |
| 112 | i | A | R | A | R | P | R | I | S | A |
| 113 | I | A | H | E | C | S | Q | V | P | S |
| 114 | I | A | H | E | C | S | Q | V | P | A |
| 115 | I | A | H | E | C | S | Q | V | S | S |
| 116 | I | A | H | E | C | S | Q | V | S | A |
| 117 | I | A | H | E | C | S | Q | I | P | S |
| 118 | I | A | H | E | C | S | Q | I | P | A |
| 119 | I | A | H | E | C | S | Q | I | S | S |
| 120 | I | A | H | E | C | S | Q | I | S | A |
| 121 | I | A | H | E | C | S | R | V | P | S |
| 122 | I | A | H | E | C | S | R | V | P | A |
| 123 | I | A | H | E | C | S | R | V | S | S |
| 124 | I | A | H | E | C | S | R | V | S | A |
| 125 | I | A | H | E | C | S | R | I | P | S |
| 126 | I | A | H | E | C | S | R | I | P | A |
| 127 | I | A | H | E | C | S | R | I | S | S |
| 128 | I | A | H | E | C | S | R | I | S | A |
| 129 | I | A | H | E | C | P | Q | V | P | S |
| 130 | I | A | H | E | C | P | Q | V | P | A |
| 131 | I | A | H | E | C | P | Q | V | S | S |
| 132 | I | A | H | E | C | P | Q | V | S | A |
| 133 | I | A | H | E | C | P | Q | I | P | S |
| 134 | I | A | H | E | C | P | Q | I | P | A |
| 135 | I | A | H | E | C | P | Q | I | S | S |
| 136 | I | A | H | E | C | P | Q | I | S | A |
| 137 | I | A | H | E | C | P | R | V | P | S |
| 138 | I | A | H | E | C | P | R | V | P | A |
| 139 | I | A | H | E | C | P | R | V | S | S |
| 140 | I | A | H | E | C | P | R | V | S | A |
| 141 | I | A | H | E | C | P | R | I | P | S |
| 142 | I | A | H | E | C | P | R | I | P | A |
| 143 | I | A | H | E | C | P | R | I | S | S |
| 144 | I | A | H | E | C | P | R | I | S | A |
| 145 | I | A | H | E | R | S | Q | V | P | S |
| 146 | I | A | H | E | R | S | Q | V | P | A |
| 147 | I | A | H | E | R | S | Q | V | S | S |
| 148 | I | A | H | E | R | S | Q | V | S | A |
| 149 | I | A | H | E | R | S | Q | I | P | S |
| 150 | I | A | H | E | R | S | Q | I | P | A |
| 151 | I | A | H | E | R | S | Q | I | S | S |
| 152 | I | A | H | E | R | S | Q | I | S | A |
| 153 | I | A | H | E | R | S | R | V | P | S |
| 154 | I | A | H | E | R | S | R | V | P | A |
| 155 | I | A | H | E | R | S | R | V | S | S |
| 156 | I | A | H | E | R | S | R | V | S | A |
| 157 | I | A | H | E | R | S | R | I | P | S |
| 158 | I | A | H | E | R | S | R | I | P | A |
| 159 | I | A | H | E | R | S | R | I | S | S |
| 160 | I | A | H | E | R | S | R | I | S | A |
| 161 | I | A | H | E | R | P | Q | V | P | S |
| 162 | I | A | H | E | R | P | Q | V | P | A |
| 163 | I | A | H | E | R | P | Q | V | S | S |
| 164 | I | A | H | E | R | P | Q | V | S | A |
| 165 | I | A | H | E | R | P | Q | I | P | S |
| 166 | I | A | H | E | R | P | Q | I | P | A |
| 167 | I | A | H | E | R | P | Q | I | S | S |
| 168 | I | A | H | E | R | P | Q | I | S | A |
| 169 | I | A | H | E | R | P | R | V | P | S |
| 170 | I | A | H | E | R | P | R | V | P | A |
| 171 | I | A | H | E | R | P | R | V | S | S |
| 172 | I | A | H | E | R | P | R | V | S | A |
| 173 | I | A | H | E | R | P | R | I | P | S |
| 174 | I | A | H | E | R | P | R | I | P | A |
| 175 | I | A | H | E | R | P | R | I | S | S |
| 176 | I | A | H | E | R | P | R | I | S | A |
| 177 | I | A | H | A | C | S | Q | V | P | S |
| 178 | I | A | H | A | C | S | Q | V | P | A |
| 179 | I | A | H | A | C | S | Q | V | S | S |
| 180 | I | A | H | A | C | S | Q | V | S | A |
| 181 | I | A | H | A | C | S | Q | I | P | S |
| 182 | I | A | H | A | C | S | Q | I | P | A |
| 183 | I | A | H | A | C | S | Q | I | S | S |
| 184 | I | A | H | A | C | S | Q | I | S | A |
| 185 | I | A | H | A | C | S | R | V | P | S |
| 186 | I | A | H | A | C | S | R | V | P | A |
| 187 | I | A | H | A | C | S | R | V | S | S |
| 188 | I | A | H | A | C | S | R | V | S | A |
| 189 | I | A | H | A | C | S | R | I | P | S |
| 190 | I | A | H | A | C | S | R | I | P | A |
| 191 | i | A | H | A | C | S | R | I | S | S |
| 192 | I | A | H | A | C | S | R | I | S | A |
| 193 | I | A | H | A | C | P | Q | V | P | S |
| 194 | I | A | H | A | C | P | Q | V | P | A |
| 195 | I | A | H | A | C | P | Q | V | S | S |
| 196 | I | A | H | A | C | P | Q | V | S | A |
| 197 | I | A | H | A | C | P | Q | I | P | S |
| 198 | I | A | H | A | C | P | Q | I | P | A |
| 199 | I | A | H | A | C | P | Q | I | S | S |
| 200 | I | A | H | A | C | P | Q | I | S | A |
| 201 | I | A | H | A | C | P | R | V | P | S |
| 202 | I | A | H | A | C | P | R | V | P | A |
| 203 | I | A | H | A | C | P | R | V | S | S |
| 204 | I | A | H | A | C | P | R | V | S | A |
| 205 | I | A | H | A | C | P | R | I | P | S |
| 206 | I | A | H | A | C | P | R | I | P | A |
| 207 | I | A | H | A | C | P | R | I | S | S |
| 208 | I | A | H | A | C | P | R | I | S | A |
| 209 | I | A | H | A | R | S | Q | V | P | S |
| 210 | I | A | H | A | R | S | Q | V | P | A |
| 211 | I | A | H | A | R | S | Q | V | S | S |
| 212 | I | A | H | A | R | S | Q | V | S | A |
| 213 | I | A | H | A | R | S | Q | I | P | S |
| 214 | I | A | H | A | R | S | Q | I | P | A |
| 215 | I | A | H | A | R | S | Q | I | S | S |
| 216 | I | A | H | A | R | S | Q | I | S | A |
| 217 | I | A | H | A | R | S | R | V | P | S |
| 218 | I | A | H | A | R | S | R | V | P | A |
| 219 | I | A | H | A | R | S | R | V | S | S |
| 220 | I | A | H | A | R | S | R | V | S | A |
| 221 | I | A | H | A | R | S | R | I | P | S |
| 222 | I | A | H | A | R | S | R | I | P | A |
| 223 | I | A | H | A | R | S | R | I | S | S |
| 224 | I | A | H | A | R | S | R | I | S | A |
| 225 | I | A | H | A | R | P | Q | V | P | S |
| 226 | I | A | H | A | R | P | Q | V | P | A |
| 227 | I | A | H | A | R | P | Q | V | S | S |
| 228 | I | A | H | A | R | P | Q | V | S | A |
| 229 | I | A | H | A | R | P | Q | I | P | S |
| 230 | 1 | A | H | A | R | P | Q | I | P | A |
| 231 | I | A | H | A | R | P | Q | I | S | S |
| 232 | I | A | H | A | R | P | Q | I | S | A |
| 233 | I | A | H | A | R | P | R | V | P | S |
| 234 | I | A | H | A | R | P | R | V | P | A |
| 235 | I | A | H | A | R | P | R | V | S | S |
| 236 | I | A | H | A | R | P | R | V | S | A |
| 237 | I | A | H | A | R | P | R | I | P | S |
| 238 | I | A | H | A | R | P | R | I | P | A |
| 239 | I | A | H | A | R | P | R | I | S | S |
| 240 | I | A | H | A | R | P | R | I | S | A |
| 241 | I | T | R | E | C | S | Q | V | P | S |
| 242 | I | T | R | E | C | S | Q | V | P | A |
| 243 | I | T | R | E | C | S | Q | V | S | S |
| 244 | I | T | R | E | C | S | Q | V | S | A |
| 245 | I | T | R | E | C | S | Q | I | P | S |
| 246 | I | T | R | E | C | S | Q | I | P | A |
| 247 | I | T | R | E | C | S | Q | I | S | S |
| 248 | I | T | R | E | C | S | Q | I | S | A |
| 249 | 1 | T | R | E | C | S | R | V | P | S |
| 250 | I | T | R | E | C | S | R | V | P | A |
| 251 | I | T | R | E | C | S | R | V | S | S |
| 252 | I | T | R | E | C | S | R | V | S | A |
| 253 | I | T | R | E | C | S | R | I | P | S |
| 254 | I | T | R | E | C | S | R | I | P | A |
| 255 | I | T | R | E | C | S | R | I | S | S |
| 256 | I | T | R | E | C | S | R | I | S | A |
| 257 | I | T | R | E | C | P | Q | V | P | S |
| 258 | I | T | R | E | C | P | Q | V | P | A |
| 259 | I | T | R | E | C | P | Q | V | S | S |
| 260 | I | T | R | E | C | P | Q | V | S | A |
| 261 | I | T | R | E | C | P | Q | I | P | S |
| 262 | I | T | R | E | C | P | Q | I | P | A |
| 263 | I | T | R | E | C | P | Q | I | S | S |
| 264 | I | T | R | E | C | P | Q | I | S | A |
| 265 | I | T | R | E | C | P | R | V | P | S |
| 266 | I | T | R | E | C | P | R | V | P | A |
| 267 | I | T | R | E | C | P | R | V | S | S |
| 268 | I | T | R | E | C | P | R | V | S | A |
| 269 | I | T | R | E | C | P | R | I | P | S |
| 270 | I | T | R | E | C | P | R | I | P | A |
| 271 | I | T | R | E | C | P | R | I | S | S |
| 272 | I | T | R | E | C | P | R | I | S | A |
| 273 | I | T | R | E | R | S | Q | V | P | S |
| 274 | I | T | R | E | R | S | Q | V | P | A |
| 275 | I | T | R | E | R | S | Q | V | S | S |
| 276 | I | T | R | E | R | S | Q | V | S | A |
| 277 | I | T | R | E | R | S | Q | I | P | S |
| 278 | I | T | R | E | R | S | Q | I | P | A |
| 279 | I | T | R | E | R | S | Q | I | S | S |
| 280 | 1 | T | R | E | R | S | Q | I | S | A |
| 281 | I | T | R | E | R | S | R | V | P | S |
| 282 | I | T | R | E | R | S | R | V | P | A |
| 283 | I | T | R | E | R | S | R | V | S | S |
| 284 | I | T | R | F | R | S | R | V | S | A |
| 285 | I | T | R | F | R | S | R | I | P | S |
| 286 | I | T | R | E | R | S | R | I | P | A |
| 287 | I | T | R | E | R | S | R | I | S | S |
| 288 | I | T | R | E | R | S | R | I | S | A |
| 289 | I | T | R | F | R | P | Q | V | P | S |
| 290 | I | T | R | E | R | P | Q | V | P | A |
| 291 | I | T | R | F | R | P | Q | V | S | S |
| 292 | I | T | R | E | R | P | Q | V | S | A |
| 293 | I | T | R | F | R | P | Q | I | P | S |
| 294 | I | T | R | E | R | P | Q | I | P | A |
| 295 | I | T | R | F | R | P | Q | I | S | S |
| 296 | I | T | R | E | R | P | Q | I | S | A |
| 297 | I | T | R | E | R | P | R | V | P | S |
| 298 | I | T | R | E | R | P | R | V | P | A |
| 299 | I | T | R | E | R | P | R | V | S | S |
| 300 | I | T | R | F | R | P | R | V | S | A |
| 301 | I | T | R | F | R | P | R | I | P | S |
| 302 | I | T | R | E | R | P | R | I | P | A |
| 303 | I | T | R | E | R | P | R | I | S | S |
| 304 | I | T | R | E | R | P | R | I | S | A |
| 305 | I | T | R | A | C | S | Q | V | P | S |
| 315 | I | T | R | A | C | S | R | V | S | S |
| 316 | I | T | R | A | C | S | R | V | S | A |
| 317 | I | T | R | A | C | S | R | I | P | S |
| 318 | I | T | R | A | C | S | R | I | P | A |
| 319 | I | T | R | A | C | S | R | I | S | S |
| 320 | I | T | R | A | C | S | R | I | S | A |
| 321 | I | T | R | A | C | P | Q | V | P | S |
| 322 | I | T | R | A | C | P | Q | V | P | A |
| 323 | I | T | R | A | C | P | Q | V | S | S |
| 324 | I | T | R | A | C | P | Q | V | S | A |
| 325 | I | T | R | A | C | P | Q | I | P | S |
| 326 | I | T | R | A | C | P | Q | I | P | A |
| 327 | I | T | R | A | C | P | Q | I | S | S |
| 328 | I | T | R | A | C | P | Q | I | S | A |
| 306 | I | T | R | A | C | S | Q | V | P | A |
| 307 | I | T | R | A | C | S | Q | V | S | S |
| 308 | I | T | R | A | C | S | Q | V | S | A |
| 309 | I | T | R | A | C | S | Q | I | P | S |
| 310 | I | T | R | A | C | S | Q | I | P | A |
| 311 | I | T | R | A | C | S | Q | I | S | S |
| 312 | I | T | R | A | C | S | Q | I | S | A |
| 313 | I | T | R | A | C | S | R | V | P | S |
| 314 | I | T | R | A | C | S | R | V | P | A |
| 329 | I | T | R | A | C | P | R | V | P | S |
| 330 | I | T | R | A | C | P | R | V | P | A |
| 331 | I | T | R | A | C | P | R | V | S | S |
| 332 | I | T | R | A | C | P | R | V | S | A |
| 333 | I | T | R | A | C | P | R | I | P | S |
| 334 | I | T | R | A | C | P | R | I | P | A |
| 335 | I | T | R | A | C | P | R | I | S | S |
| 336 | I | T | R | A | C | P | R | I | S | A |
| 337 | I | T | R | A | R | S | Q | V | P | S |
| 338 | I | T | R | A | R | S | Q | V | P | A |
| 339 | I | T | R | A | R | S | Q | V | S | S |
| 340 | I | T | R | A | R | S | Q | V | S | A |
| 341 | I | T | R | A | R | S | Q | I | P | S |
| 342 | I | T | R | A | R | S | Q | I | P | A |
| 343 | I | T | R | A | R | S | Q | I | S | S |
| 344 | I | T | R | A | R | S | Q | I | S | A |
| 345 | I | T | R | A | R | S | R | V | P | S |
| 346 | I | T | R | A | R | S | R | V | P | A |
| 347 | I | T | R | A | R | S | R | V | S | S |
| 348 | I | T | R | A | R | S | R | V | S | A |
| 349 | I | T | R | A | R | S | R | I | P | S |
| 350 | I | T | R | A | R | S | R | I | P | A |
| 351 | I | T | R | A | R | S | R | I | S | S |
| 352 | I | T | R | A | R | S | R | I | S | A |
| 353 | I | T | R | A | R | P | Q | V | P | S |
| 354 | I | T | R | A | R | P | Q | V | P | A |
| 355 | I | T | R | A | R | P | Q | V | S | S |
| 356 | I | T | R | A | R | P | Q | V | S | A |
| 357 | I | T | R | A | R | P | Q | I | P | S |
| 358 | I | T | R | A | R | P | Q | I | P | A |
| 359 | I | T | R | A | R | P | Q | I | S | S |
| 360 | I | T | R | A | R | P | Q | I | S | A |
| 361 | I | T | R | A | R | P | R | V | P | S |
| 362 | I | T | R | A | R | P | R | V | P | A |
| 363 | I | T | R | A | R | P | R | V | S | S |
| 364 | I | T | R | A | R | P | R | V | S | A |
| 365 | I | T | R | A | R | P | R | I | P | S |
| 366 | I | T | R | A | R | P | R | I | P | A |
| 367 | I | T | R | A | R | P | R | I | S | S |
| 368 | I | T | R | A | R | P | R | I | S | A |
| 369 | I | T | H | E | C | S | Q | V | P | S |
| 370 | I | T | H | E | C | S | Q | V | P | A |
| 371 | I | T | H | E | C | S | Q | V | S | S |
| 372 | I | T | H | E | C | S | Q | V | S | A |
| 373 | I | T | H | E | C | S | Q | I | P | S |
| 374 | I | T | H | E | C | S | Q | I | P | A |
| 375 | I | T | H | E | C | S | Q | I | S | S |
| 376 | I | T | H | E | C | S | Q | I | S | A |
| 377 | I | T | H | E | C | S | R | V | P | S |
| 378 | I | T | H | E | C | S | R | V | P | A |
| 379 | I | T | H | E | C | S | R | V | S | S |
| 380 | I | T | H | E | C | S | R | V | S | A |
| 381 | I | T | H | E | C | S | R | I | P | S |
| 382 | I | T | H | E | C | S | R | I | P | A |
| 383 | I | T | H | E | C | S | R | I | S | S |
| 384 | I | T | H | E | C | S | R | I | S | A |
| 385 | I | T | H | E | C | P | Q | V | P | S |
| 386 | I | T | H | E | C | P | Q | V | P | A |
| 387 | I | T | H | E | C | P | Q | V | S | S |
| 388 | I | T | H | E | C | P | Q | V | S | A |
| 389 | I | T | H | E | C | P | Q | I | P | S |
| 390 | I | T | H | E | C | P | Q | I | P | A |
| 391 | I | T | H | E | C | P | Q | I | S | S |
| 392 | I | T | H | E | C | P | Q | I | S | A |
| 393 | I | T | H | E | C | P | R | V | P | S |
| 394 | I | T | H | E | C | P | R | V | P | A |
| 395 | I | T | H | E | C | P | R | V | S | S |
| 396 | I | T | H | E | C | P | R | V | S | A |
| 397 | I | T | H | E | C | P | R | I | P | S |
| 398 | I | T | H | E | C | P | R | I | P | A |
| 399 | I | T | H | E | C | P | R | I | S | S |
| 400 | I | T | H | E | C | P | R | I | S | A |
| 401 | I | T | H | E | R | S | Q | V | P | S |
| 402 | I | T | H | E | R | S | Q | V | P | A |
| 403 | I | T | H | E | R | S | Q | V | S | S |
| 404 | I | T | H | E | R | S | Q | V | S | A |
| 405 | I | T | H | E | R | S | Q | I | P | S |
| 406 | I | T | H | E | R | S | Q | I | P | A |
| 407 | I | T | H | E | R | S | Q | I | S | S |
| 408 | I | T | H | E | R | S | Q | I | S | A |
| 409 | I | T | H | E | R | S | R | V | P | S |
| 410 | I | T | H | E | R | S | R | V | P | A |
| 411 | I | T | H | E | R | S | R | V | S | S |
| 412 | I | T | H | E | R | S | R | V | S | A |
| 413 | I | T | H | E | R | S | R | I | P | S |
| 414 | I | T | H | E | R | S | R | I | P | A |
| 415 | I | T | H | E | R | S | R | I | S | S |
| 416 | I | T | H | E | R | S | R | I | S | A |
| 417 | I | T | H | E | R | P | Q | V | P | S |
| 418 | I | T | H | E | R | P | Q | V | P | A |
| 419 | I | T | H | E | R | P | Q | V | S | S |
| 420 | I | T | H | E | R | P | Q | V | S | A |
| 421 | I | T | H | E | R | P | Q | I | P | S |
| 422 | I | T | H | E | R | P | Q | I | P | A |
| 423 | I | T | H | E | R | P | Q | I | S | S |
| 424 | I | T | H | E | R | P | Q | I | S | A |
| 425 | I | T | H | E | R | P | R | V | P | S |
| 426 | I | T | H | E | R | P | R | V | P | A |
| 427 | I | T | H | E | R | P | R | V | S | S |
| 428 | I | T | H | E | R | P | R | V | S | A |
| 429 | I | T | H | E | R | P | R | I | P | S |
| 430 | I | T | H | E | R | P | R | I | P | A |
| 431 | I | T | H | E | R | P | R | I | S | S |
| 432 | I | T | H | E | R | P | R | I | S | A |
| 433 | I | T | H | A | C | S | Q | V | P | S |
| 434 | I | T | H | A | C | S | Q | V | P | A |
| 435 | I | T | H | A | C | S | Q | V | S | S |
| 436 | I | T | H | A | C | S | Q | V | S | A |
| 437 | I | T | H | A | C | S | Q | I | P | S |
| 438 | I | T | H | A | C | S | Q | I | P | A |
| 439 | I | T | H | A | C | S | Q | I | S | S |
| 440 | I | T | H | A | C | S | Q | I | S | A |
| 441 | I | T | H | A | C | S | R | V | P | S |
| 442 | I | T | H | A | C | S | R | V | P | A |
| 443 | I | T | H | A | C | S | R | V | S | S |
| 444 | I | T | H | A | C | S | R | V | S | A |
| 445 | I | T | H | A | C | S | R | I | P | S |
| 446 | I | T | H | A | C | S | R | I | P | A |
| 447 | I | T | H | A | C | S | R | I | S | S |
| 448 | I | T | H | A | C | S | R | I | S | A |
| 449 | I | T | H | A | C | P | Q | V | P | S |
| 450 | I | T | H | A | C | P | Q | V | P | A |
| 451 | I | T | H | A | C | P | Q | V | S | S |
| 452 | I | T | H | A | C | P | Q | V | S | A |
| 453 | I | T | H | A | C | P | Q | I | P | S |
| 454 | I | T | H | A | C | P | Q | I | P | A |
| 455 | I | T | H | A | C | P | Q | I | S | S |
| 456 | I | T | H | A | C | P | Q | I | S | A |
| 457 | I | T | H | A | C | P | R | V | P | S |
| 458 | I | T | H | A | C | P | R | V | P | A |
| 459 | I | T | H | A | C | P | R | V | S | S |
| 460 | I | T | H | A | C | P | R | V | S | A |
| 461 | I | T | H | A | C | P | R | I | P | S |
| 462 | I | T | H | A | C | P | R | I | P | A |
| 463 | I | T | H | A | C | P | R | I | S | S |
| 464 | I | T | H | A | C | P | R | I | S | A |
| 465 | I | T | H | A | R | S | Q | V | P | S |
| 466 | I | T | H | A | R | S | Q | V | P | A |
| 467 | I | T | H | A | R | S | Q | V | S | S |
| 468 | I | T | H | A | R | S | Q | V | S | A |
| 469 | I | T | H | A | R | S | Q | I | P | S |
| 470 | I | T | H | A | R | S | Q | I | P | A |
| 471 | I | T | H | A | R | S | Q | I | S | S |
| 472 | I | T | H | A | R | S | Q | I | S | A |
| 473 | I | T | H | A | R | S | R | V | P | S |
| 474 | I | T | H | A | R | S | R | V | P | A |
| 475 | I | T | H | A | R | S | R | V | S | S |
| 476 | I | T | H | A | R | S | R | V | S | A |
| 477 | I | T | H | A | R | S | R | I | P | S |
| 478 | I | T | H | A | R | S | R | I | P | A |
| 479 | I | T | H | A | R | S | R | I | S | S |
| 480 | I | T | H | A | R | S | R | I | S | A |
| 481 | I | T | H | A | R | P | Q | V | P | S |
| 482 | I | T | H | A | R | P | Q | V | P | A |
| 483 | I | T | H | A | R | P | Q | V | S | S |
| 484 | I | T | H | A | R | P | Q | V | S | A |
| 485 | I | T | H | A | R | P | Q | I | P | S |
| 486 | I | T | H | A | R | P | Q | I | P | A |
| 487 | I | T | H | A | R | P | Q | I | S | S |
| 488 | I | T | H | A | R | P | Q | I | S | A |
| 489 | I | T | H | A | R | P | R | V | P | S |
| 490 | I | T | H | A | R | P | R | V | P | A |
| 491 | I | T | H | A | R | P | R | V | S | S |
| 492 | I | T | H | A | R | P | R | V | S | A |
| 493 | I | T | H | A | R | P | R | I | P | S |
| 494 | I | T | H | A | R | P | R | I | P | A |
| 495 | I | T | H | A | R | P | R | I | S | S |
| 496 | I | T | H | A | R | P | R | I | S | A |
| 497 | V | A | R | E | C | S | Q | V | P | A |
| 498 | V | A | R | F | C | S | Q | V | S | S |
| 499 | V | A | R | E | C | S | Q | V | S | A |
| 500 | V | A | R | F | C | S | Q | I | P | S |
| 501 | V | A | R | F | C | S | Q | I | P | A |
| 502 | V | A | R | E | C | S | Q | I | S | S |
| 503 | V | A | R | E | C | S | Q | I | S | A |
| 504 | V | A | R | E | C | S | R | V | P | A |
| 505 | V | A | R | E | C | S | R | V | S | S |
| 506 | V | A | R | E | C | S | R | V | S | A |
| 507 | V | A | R | E | C | S | R | I | P | S |
| 508 | V | A | R | E | C | S | R | I | P | A |
| 509 | V | A | R | E | C | S | R | I | S | S |
| 510 | V | A | R | E | C | S | R | I | S | A |
| 511 | V | A | R | E | C | P | Q | V | P | A |
| 512 | V | A | R | E | C | P | Q | V | S | S |
| 513 | V | A | R | E | C | P | Q | V | S | A |
| 514 | V | A | R | F | C | P | Q | I | P | S |
| 515 | V | A | R | F | C | P | Q | I | P | A |
| 516 | V | A | R | E | C | P | Q | I | S | S |
| 517 | V | A | R | E | C | P | Q | I | S | A |
| 518 | V | A | R | E | C | P | R | V | P | A |
| 519 | V | A | R | E | C | P | R | V | S | S |
| 520 | V | A | R | E | C | P | R | V | S | A |
| 521 | V | A | R | E | C | P | R | I | P | S |
| 522 | V | A | R | E | C | P | R | I | P | A |
| 523 | V | A | R | E | C | P | R | I | S | S |
| 524 | V | A | R | E | C | P | R | I | S | A |
| 525 | V | A | R | E | R | S | Q | V | P | A |
| 526 | V | A | R | E | R | S | Q | V | S | S |
| 527 | V | A | R | E | R | S | Q | V | S | A |
| 528 | V | A | R | F | R | S | Q | I | P | S |
| 529 | V | A | R | F | R | S | Q | I | P | A |
| 530 | V | A | R | F | R | S | Q | I | S | S |
| 531 | V | A | R | E | R | S | Q | I | S | A |
| 532 | V | A | R | E | R | S | R | V | P | A |
| 533 | V | A | R | E | R | S | R | V | S | S |
| 534 | V | A | R | E | R | S | R | V | S | A |
| 535 | V | A | R | E | R | S | R | I | P | S |
| 536 | V | A | R | E | R | S | R | I | P | A |
| 537 | V | A | R | E | R | S | R | I | S | S |
| 538 | V | A | R | E | R | S | R | I | S | A |
| 539 | V | A | R | E | R | P | Q | V | P | A |
| 540 | V | A | R | E | R | P | Q | V | S | S |
| 541 | V | A | R | E | R | P | Q | V | S | A |
| 542 | V | A | R | E | R | P | Q | I | P | S |
| 543 | V | A | R | E | R | P | Q | I | P | A |
| 544 | V | A | R | E | R | P | Q | I | S | S |
| 545 | V | A | R | E | R | P | Q | I | S | A |
| 546 | V | A | R | E | R | P | R | V | P | A |
| 547 | V | A | R | E | R | P | R | V | S | S |
| 548 | V | A | R | E | R | P | R | V | S | A |
| 549 | V | A | R | E | R | P | R | I | P | S |
| 550 | V | A | R | E | R | P | R | I | P | A |
| 551 | V | A | R | E | R | P | R | I | S | S |
| 552 | V | A | R | E | R | P | R | I | S | A |
| 553 | V | A | R | A | C | S | Q | V | P | A |
| 554 | V | A | R | A | C | S | Q | V | S | S |
| 555 | V | A | R | A | C | S | Q | V | S | A |
| 556 | V | A | R | A | C | S | Q | I | P | S |
| 557 | V | A | R | A | C | S | Q | I | P | A |
| 558 | V | A | R | A | C | S | Q | I | S | S |
| 559 | V | A | R | A | C | S | Q | I | S | A |
| 560 | V | A | R | A | C | S | R | V | P | A |
| 561 | V | A | R | A | C | S | R | V | S | S |
| 562 | V | A | R | A | C | S | R | V | S | A |
| 563 | V | A | R | A | C | S | R | I | P | S |
| 564 | V | A | R | A | C | S | R | I | P | A |
| 565 | V | A | R | A | C | S | R | I | S | S |
| 566 | V | A | R | A | C | S | R | I | S | A |
| 567 | V | A | R | A | C | P | Q | V | P | A |
| 568 | V | A | R | A | C | P | Q | V | S | S |
| 569 | V | A | R | A | C | P | Q | V | S | A |
| 570 | V | A | R | A | C | P | Q | I | P | S |
| 571 | V | A | R | A | C | P | Q | I | P | A |
| 572 | V | A | R | A | C | P | Q | I | S | S |
| 573 | V | A | R | A | C | P | Q | I | S | A |
| 574 | V | A | R | A | C | P | R | V | P | A |
| 575 | V | A | R | A | C | P | R | V | S | S |
| 576 | V | A | R | A | C | P | R | V | S | A |
| 577 | V | A | R | A | C | P | R | I | P | S |
| 578 | V | A | R | A | C | P | R | I | P | A |
| 579 | V | A | R | A | C | P | R | I | S | S |
| 580 | V | A | R | A | C | P | R | I | S | A |
| 581 | V | A | R | A | R | S | Q | V | P | A |
| 582 | V | A | R | A | R | S | Q | V | S | S |
| 583 | V | A | R | A | R | S | Q | V | S | A |
| 584 | V | A | R | A | R | S | Q | I | P | S |
| 585 | V | A | R | A | R | S | Q | I | P | A |
| 586 | V | A | R | A | R | S | Q | I | S | S |
| 587 | V | A | R | A | R | S | Q | I | S | A |
| 588 | V | A | R | A | R | S | R | V | P | A |
| 589 | V | A | R | A | R | S | R | V | S | S |
| 590 | V | A | R | A | R | S | R | V | S | A |
| 591 | V | A | R | A | R | S | R | I | P | S |
| 592 | V | A | R | A | R | S | R | I | P | A |
| 593 | V | A | R | A | R | S | R | I | S | S |
| 594 | V | A | R | A | R | S | R | I | S | A |
| 595 | V | A | R | A | R | P | Q | V | P | A |
| 596 | V | A | R | A | R | P | Q | V | S | S |
| 597 | V | A | R | A | R | P | Q | V | S | A |
| 598 | V | A | R | A | R | P | Q | I | P | S |
| 599 | V | A | R | A | R | P | Q | I | P | A |
| 600 | V | A | R | A | R | P | Q | I | S | S |
| 601 | V | A | R | A | R | P | Q | I | S | A |
| 602 | V | A | R | A | R | P | R | V | P | A |
| 603 | V | A | R | A | R | P | R | V | S | S |
| 604 | V | A | R | A | R | P | R | V | S | A |
| 605 | V | A | R | A | R | P | R | I | P | S |
| 606 | V | A | R | A | R | P | R | I | P | A |
| 607 | V | A | R | A | R | P | R | I | S | S |
| 608 | V | A | R | A | R | P | R | I | S | A |
| 609 | V | A | H | E | C | S | Q | V | P | S |
| 610 | V | A | H | E | C | S | Q | V | P | A |
| 611 | V | A | H | E | C | S | Q | V | S | S |
| 612 | V | A | H | E | C | S | Q | V | S | A |
| 613 | V | A | H | E | C | S | Q | I | P | S |
| 614 | V | A | H | E | C | S | Q | I | P | A |
| 615 | V | A | H | E | C | S | Q | I | S | S |
| 616 | V | A | H | E | C | S | Q | I | S | A |
| 617 | V | A | H | E | C | S | R | V | P | S |
| 618 | V | A | H | E | C | S | R | V | P | A |
| 619 | V | A | H | E | C | S | R | V | S | S |
| 620 | V | A | H | E | C | S | R | V | S | A |
| 621 | V | A | H | E | C | S | R | I | P | S |
| 622 | V | A | H | E | C | S | R | I | P | A |
| 623 | V | A | H | E | C | S | R | I | S | S |
| 624 | V | A | H | E | C | S | R | I | S | A |
| 625 | V | A | H | E | C | P | Q | V | P | S |
| 626 | V | A | H | E | C | P | Q | V | P | A |
| 627 | V | A | H | E | C | P | Q | V | S | S |
| 628 | V | A | H | E | C | P | Q | V | S | A |
| 629 | V | A | H | E | C | P | Q | I | P | S |
| 630 | V | A | H | E | C | P | Q | I | P | A |
| 631 | V | A | H | E | C | P | C | I | S | S |
| 632 | V | A | H | E | C | P | Q | I | S | A |
| 633 | V | A | H | E | C | P | R | V | P | S |
| 634 | V | A | H | E | C | P | R | V | P | A |
| 635 | V | A | H | E | C | P | R | V | S | S |
| 636 | V | A | H | E | C | P | R | V | S | A |
| 637 | V | A | H | E | C | P | R | I | P | S |
| 638 | V | A | H | E | C | P | R | I | P | A |
| 639 | V | A | H | E | C | P | R | I | S | S |
| 640 | V | A | H | E | C | P | R | I | S | A |
| 641 | V | A | H | E | R | S | Q | V | P | S |
| 642 | V | A | H | E | R | S | Q | V | P | A |
| 643 | V | A | H | E | R | S | Q | V | S | S |
| 644 | V | A | H | E | R | S | Q | V | S | A |
| 645 | V | A | H | E | R | S | Q | I | P | S |
| 646 | V | A | H | E | R | S | Q | I | P | A |
| 647 | V | A | H | E | R | S | Q | I | S | S |
| 648 | V | A | H | E | R | S | Q | I | S | A |
| 649 | V | A | H | E | R | S | R | V | P | S |
| 650 | V | A | H | E | R | S | R | V | P | A |
| 651 | V | A | H | E | R | S | R | V | S | S |
| 652 | V | A | H | E | R | S | R | V | S | A |
| 653 | V | A | H | E | R | S | R | I | P | S |
| 654 | V | A | H | E | R | S | R | I | P | A |
| 655 | V | A | H | E | R | S | R | I | S | S |
| 656 | V | A | H | E | R | S | R | I | S | A |
| 657 | V | A | H | E | R | P | Q | V | P | S |
| 658 | V | A | H | E | R | P | Q | V | P | A |
| 659 | V | A | H | E | R | P | Q | V | S | S |
| 660 | V | A | H | E | R | P | Q | V | S | A |
| 661 | V | A | H | E | R | P | Q | I | P | S |
| 662 | V | A | H | E | R | P | Q | I | P | A |
| 663 | V | A | H | E | R | P | Q | I | S | S |
| 664 | V | A | H | E | R | P | Q | I | S | A |
| 665 | V | A | H | E | R | P | R | V | P | S |
| 666 | V | A | H | E | R | P | R | V | P | A |
| 667 | V | A | H | E | R | P | R | V | S | S |
| 668 | V | A | H | E | R | P | R | V | S | A |
| 669 | V | A | H | F | R | P | R | I | P | S |
| 670 | V | A | H | E | R | P | R | I | P | A |
| 671 | V | A | H | F | R | P | R | I | S | S |
| 672 | V | A | H | E | R | P | R | I | S | A |
| 673 | V | A | H | A | C | S | Q | V | P | S |
| 674 | V | A | H | A | C | S | Q | V | P | A |
| 675 | V | A | H | A | C | S | Q | V | S | S |
| 676 | V | A | H | A | C | S | Q | V | S | A |
| 677 | V | A | H | A | C | S | Q | I | P | S |
| 678 | V | A | H | A | C | S | Q | I | P | A |
| 679 | V | A | H | A | C | S | Q | I | S | S |
| 680 | V | A | H | A | C | S | Q | I | S | A |
| 681 | V | A | H | A | C | S | R | V | P | S |
| 682 | V | A | H | A | C | S | R | V | P | A |
| 683 | V | A | H | A | C | S | R | V | S | S |
| 684 | V | A | H | A | C | S | R | V | S | A |
| 685 | V | A | H | A | C | S | R | I | P | S |
| 686 | V | A | H | A | C | S | R | I | P | A |
| 687 | V | A | H | A | C | S | R | I | S | S |
| 688 | V | A | H | A | C | S | R | I | S | A |
| 689 | V | A | H | A | C | P | Q | V | P | S |
| 690 | V | A | H | A | C | P | Q | V | P | A |
| 691 | V | A | H | A | C | P | Q | V | S | S |
| 692 | V | A | H | A | C | P | Q | V | S | A |
| 693 | V | A | H | A | C | P | Q | I | P | S |
| 694 | V | A | H | A | C | P | Q | I | P | A |
| 695 | V | A | H | A | C | P | Q | I | S | S |
| 696 | V | A | H | A | C | P | Q | I | S | A |
| 697 | V | A | H | A | C | P | R | V | P | S |
| 698 | V | A | H | A | C | P | R | V | P | A |
| 699 | V | A | H | A | C | P | R | V | S | S |
| 700 | V | A | H | A | C | P | R | V | S | A |
| 701 | V | A | H | A | C | P | R | I | P | S |
| 702 | V | A | H | A | C | P | R | I | P | A |
| 703 | V | A | H | A | C | P | R | I | S | S |
| 704 | V | A | H | A | C | P | R | I | S | A |
| 705 | V | A | H | A | R | S | Q | V | P | S |
| 706 | V | A | H | A | R | S | Q | V | P | A |
| 707 | V | A | H | A | R | S | Q | V | S | S |
| 708 | V | A | H | A | R | S | Q | V | S | A |
| 709 | V | A | H | A | R | S | Q | I | P | S |
| 710 | V | A | H | A | R | S | Q | I | P | A |
| 711 | V | A | H | A | R | S | Q | I | S | S |
| 712 | V | A | H | A | R | S | Q | I | S | A |
| 713 | V | A | H | A | R | S | R | V | P | S |
| 714 | V | A | H | A | R | S | R | V | P | A |
| 715 | V | A | H | A | R | S | R | V | S | S |
| 716 | V | A | H | A | R | S | R | V | S | A |
| 717 | V | A | H | A | R | S | R | I | P | S |
| 718 | V | A | H | A | R | S | R | I | P | A |
| 719 | V | A | H | A | R | S | R | I | S | S |
| 720 | V | A | H | A | R | S | R | I | S | A |
| 721 | V | A | H | A | R | P | Q | V | P | S |
| 722 | V | A | H | A | R | P | Q | V | P | A |
| 723 | V | A | H | A | R | P | Q | V | S | S |
| 724 | V | A | H | A | R | P | Q | V | S | A |
| 725 | V | A | H | A | R | P | Q | I | P | S |
| 726 | V | A | H | A | R | P | Q | I | P | A |
| 727 | V | A | H | A | R | P | Q | I | S | S |
| 728 | V | A | H | A | R | P | Q | I | S | A |
| 729 | V | A | H | A | R | P | R | V | P | S |
| 730 | V | A | H | A | R | P | R | V | P | A |
| 731 | V | A | H | A | R | P | R | V | S | S |
| 732 | V | A | H | A | R | P | R | V | S | A |
| 733 | V | A | H | A | R | P | R | I | P | S |
| 734 | V | A | H | A | R | P | R | I | P | A |
| 735 | V | A | H | A | R | P | R | I | S | S |
| 736 | V | A | H | A | R | P | R | I | S | A |
| 737 | V | T | R | E | C | S | Q | V | P | S |
| 738 | V | T | R | E | C | S | Q | V | P | A |
| 739 | V | T | R | E | C | S | Q | V | S | S |
| 740 | V | T | R | E | C | S | Q | V | S | A |
| 741 | V | T | R | E | C | S | Q | I | P | S |
| 742 | V | T | R | E | C | S | Q | I | P | A |
| 743 | V | T | R | E | C | S | Q | I | S | S |
| 744 | V | T | R | E | C | S | Q | V | S | A |
| 745 | V | T | R | E | C | S | R | V | P | S |
| 746 | V | T | R | E | C | S | R | V | P | A |
| 747 | V | T | R | E | C | S | R | V | S | S |
| 748 | V | T | R | F | C | S | R | V | S | A |
| 749 | V | T | R | E | C | S | R | I | P | S |
| 750 | V | T | R | E | C | S | R | I | P | A |
| 751 | V | T | R | E | C | S | R | I | S | S |
| 752 | V | T | R | E | C | S | R | I | S | A |
| 753 | V | T | R | E | C | P | Q | V | P | S |
| 754 | V | T | R | E | C | P | Q | V | P | A |
| 755 | V | T | R | E | C | P | Q | V | S | S |
| 756 | V | T | R | E | C | P | Q | V | S | A |
| 757 | V | T | R | F | C | P | Q | I | P | S |
| 758 | V | T | R | E | C | P | Q | I | P | A |
| 759 | V | T | R | E | C | P | Q | I | S | S |
| 760 | V | T | R | E | C | P | Q | I | S | A |
| 761 | V | T | R | E | C | P | R | V | P | S |
| 762 | V | T | R | E | C | P | R | V | P | A |
| 763 | V | T | R | F | C | P | R | V | S | S |
| 764 | V | T | R | E | C | P | R | V | S | A |
| 765 | V | T | R | E | C | P | R | I | P | S |
| 766 | V | T | R | F | C | P | R | I | P | A |
| 767 | V | T | R | E | C | P | R | I | S | S |
| 768 | V | T | R | E | C | P | R | I | S | A |
| 769 | V | T | R | E | R | S | Q | V | P | S |
| 770 | V | T | R | E | R | S | Q | V | P | A |
| 771 | V | T | R | E | R | S | Q | V | S | S |
| 772 | V | T | R | E | R | S | Q | V | S | A |
| 773 | V | T | R | E | R | S | Q | I | P | S |
| 774 | V | T | R | E | R | S | Q | I | P | A |
| 775 | V | T | R | E | R | S | Q | I | S | S |
| 776 | V | T | R | E | R | S | Q | I | S | A |
| 777 | V | T | R | E | R | S | R | V | P | S |
| 778 | V | T | R | E | R | S | R | V | P | A |
| 779 | V | T | R | F | R | S | R | V | S | S |
| 780 | V | T | R | E | R | S | R | V | S | A |
| 781 | V | T | R | E | R | S | R | I | P | S |
| 782 | V | T | R | E | R | S | R | I | P | A |
| 783 | V | T | R | E | R | S | R | I | S | S |
| 784 | V | T | R | E | R | S | R | I | S | A |
| 785 | V | T | R | E | R | P | Q | V | P | S |
| 786 | V | T | R | E | R | P | Q | V | P | A |
| 787 | V | T | R | E | R | P | Q | V | S | S |
| 788 | V | T | R | E | R | P | Q | V | S | A |
| 789 | V | T | R | E | R | P | Q | I | P | S |
| 790 | V | T | R | E | R | P | Q | I | P | A |
| 791 | V | T | R | F | R | P | Q | I | S | S |
| 792 | V | T | R | E | R | P | Q | I | S | A |
| 793 | V | T | R | E | R | P | R | V | P | S |
| 794 | V | T | R | F | R | P | R | V | P | A |
| 795 | V | T | R | E | R | P | R | V | S | S |
| 796 | V | T | R | E | R | P | R | V | S | A |
| 797 | V | T | R | E | R | P | R | I | P | S |
| 798 | V | T | R | E | R | P | R | I | P | A |
| 799 | V | T | R | E | R | P | R | I | S | S |
| 800 | V | T | R | E | R | P | R | I | S | A |
| 801 | V | T | R | A | C | S | Q | V | P | S |
| 802 | V | T | R | A | C | S | Q | V | P | A |
| 803 | V | T | R | A | C | S | Q | V | S | S |
| 804 | V | T | R | A | C | S | Q | V | S | A |
| 805 | V | T | R | A | C | S | Q | I | P | S |
| 806 | V | T | R | A | C | S | Q | I | P | A |
| 807 | V | T | R | A | C | S | Q | I | S | S |
| 808 | V | T | R | A | C | S | Q | I | S | A |
| 809 | V | T | R | A | C | S | R | V | P | S |
| 810 | V | T | R | A | C | S | R | V | P | A |
| 811 | V | T | R | A | C | S | R | V | S | S |
| 812 | V | T | R | A | C | S | R | V | S | A |
| 813 | V | T | R | A | C | S | R | I | P | S |
| 814 | V | T | R | A | C | S | R | I | P | A |
| 815 | V | T | R | A | C | S | R | I | S | S |
| 816 | V | T | R | A | C | S | R | I | S | A |
| 817 | V | T | R | A | C | P | Q | V | P | S |
| 818 | V | T | R | A | C | P | Q | V | P | A |
| 819 | V | T | R | A | C | P | Q | V | S | S |
| 820 | V | T | R | A | C | P | Q | V | S | A |
| 821 | V | T | R | A | C | P | Q | I | P | S |
| 822 | V | T | R | A | C | P | Q | I | P | A |
| 823 | V | T | R | A | C | P | Q | I | S | S |
| 824 | V | i | R | A | C | P | Q | I | S | A |
| 825 | V | T | R | A | C | P | R | V | P | S |
| 826 | V | T | R | A | C | P | R | V | P | A |
| 827 | V | T | R | A | C | P | R | V | S | S |
| 828 | V | T | R | A | C | P | R | V | S | A |
| 829 | V | T | R | A | C | P | R | I | P | S |
| 830 | V | T | R | A | C | P | R | I | P | A |
| 831 | V | T | R | A | C | P | R | I | S | S |
| 832 | V | T | R | A | C | P | R | I | S | A |
| 833 | V | T | R | A | R | S | Q | V | P | S |
| 834 | V | T | R | A | R | S | Q | V | P | A |
| 835 | V | T | R | A | R | S | Q | V | S | S |
| 836 | V | T | R | A | R | S | Q | V | S | A |
| 837 | V | T | R | A | R | S | Q | I | P | S |
| 838 | V | T | R | A | R | S | Q | I | P | A |
| 839 | V | T | R | A | R | S | Q | I | S | S |
| 840 | V | T | R | A | R | S | Q | I | S | A |
| 841 | V | T | R | A | R | S | R | V | P | S |
| 842 | V | T | R | A | R | S | R | V | P | A |
| 843 | V | T | R | A | R | S | R | V | S | S |
| 844 | V | T | R | A | R | S | R | V | S | A |
| 845 | V | T | R | A | R | S | R | I | P | S |
| 846 | V | T | R | A | R | S | R | I | P | A |
| 847 | V | T | R | A | R | S | R | I | S | S |
| 848 | V | T | R | A | R | S | R | I | S | A |
| 849 | V | T | R | A | R | P | Q | V | P | S |
| 850 | V | T | R | A | R | P | Q | V | P | A |
| 851 | V | T | R | A | R | P | Q | V | S | S |
| 852 | V | T | R | A | R | P | Q | V | S | A |
| 853 | V | T | R | A | R | P | Q | I | P | S |
| 854 | V | T | R | A | R | P | Q | I | P | A |
| 855 | V | T | R | A | R | P | Q | I | S | S |
| 856 | V | T | R | A | R | P | Q | I | S | A |
| 857 | V | T | R | A | R | P | R | V | P | S |
| 858 | V | T | R | A | R | P | R | V | P | A |
| 859 | V | T | R | A | R | P | R | V | S | S |
| 860 | V | T | R | A | R | P | R | V | S | A |
| 861 | V | T | R | A | R | P | R | I | P | S |
| 862 | V | T | R | A | R | P | R | I | P | A |
| 863 | V | T | R | A | R | P | R | I | S | S |
| 864 | V | T | R | A | R | P | R | I | S | A |
| 865 | V | T | H | F | C | S | Q | V | P | S |
| 866 | V | T | H | F | C | S | Q | V | P | A |
| 867 | V | T | H | E | C | S | Q | V | S | S |
| 868 | V | T | H | E | C | S | Q | V | S | A |
| 869 | V | T | H | E | C | S | Q | I | P | S |
| 870 | V | T | H | E | C | S | Q | I | P | A |
| 871 | V | T | H | E | C | S | Q | I | S | S |
| 872 | V | T | H | E | C | S | Q | I | S | A |
| 873 | V | T | H | E | C | S | R | V | P | S |
| 874 | V | T | H | E | C | S | R | V | P | A |
| 875 | V | T | H | E | C | S | R | V | S | S |
| 876 | V | T | H | E | C | S | R | V | S | A |
| 877 | V | T | H | E | C | S | R | I | P | S |
| 878 | V | T | H | E | C | S | R | I | P | A |
| 879 | V | T | H | E | C | S | R | I | S | S |
| 880 | V | T | H | F | C | S | R | I | S | A |
| 881 | V | T | H | E | C | P | Q | V | P | S |
| 882 | V | T | H | E | C | P | Q | V | P | A |
| 883 | V | T | H | F | C | P | Q | V | S | S |
| 884 | V | T | H | E | C | P | Q | V | S | A |
| 885 | V | T | H | E | C | P | Q | I | P | S |
| 886 | V | T | H | E | C | P | Q | I | P | A |
| 887 | V | T | H | E | C | P | Q | I | S | S |
| 888 | V | T | H | E | C | P | Q | I | S | A |
| 889 | V | T | H | F | C | P | R | V | P | S |
| 890 | V | T | H | F | C | P | R | V | P | A |
| 891 | V | T | H | E | C | P | R | V | S | S |
| 892 | V | T | H | E | C | P | R | V | S | A |
| 893 | V | T | H | E | C | P | R | I | P | S |
| 894 | V | T | H | E | C | P | R | I | P | A |
| 895 | V | T | H | E | C | P | R | I | S | S |
| 896 | V | T | H | E | C | P | R | I | S | A |
| 897 | V | T | H | E | R | S | Q | V | P | S |
| 898 | V | T | H | E | R | S | Q | V | P | A |
| 899 | V | T | H | E | R | S | Q | V | S | S |
| 900 | V | T | H | E | R | S | Q | V | S | A |
| 901 | V | T | H | E | R | S | Q | I | P | S |
| 902 | V | T | H | E | R | S | Q | I | P | A |
| 903 | V | T | H | E | R | S | Q | I | S | S |
| 904 | V | T | H | E | R | S | Q | I | S | A |
| 905 | V | T | H | E | R | S | R | V | P | S |
| 906 | V | T | H | E | R | S | R | V | P | A |
| 907 | V | T | H | E | R | S | R | V | S | S |
| 908 | V | T | H | E | R | S | R | V | S | A |
| 909 | V | T | H | E | R | S | R | I | P | S |
| 910 | V | T | H | E | R | S | R | I | P | A |
| 911 | V | T | H | E | R | S | R | I | S | S |
| 912 | V | T | H | E | R | S | R | I | S | A |
| 913 | V | T | H | E | R | P | Q | V | P | S |
| 914 | V | T | H | E | R | P | Q | V | P | A |
| 915 | V | T | H | E | R | P | Q | V | S | S |
| 916 | V | T | H | E | R | P | Q | V | S | A |
| 917 | V | T | H | E | R | P | Q | I | P | S |
| 918 | V | T | H | E | R | P | Q | I | P | A |
| 919 | V | T | H | E | R | P | Q | I | S | S |
| 920 | V | T | H | E | R | P | Q | I | S | A |
| 921 | V | T | H | E | R | P | R | V | P | S |
| 922 | V | T | H | E | R | P | R | V | P | A |
| 923 | V | T | H | E | R | P | R | V | S | S |
| 924 | V | T | H | E | R | P | R | V | S | A |
| 925 | V | T | H | E | R | P | R | I | P | S |
| 926 | V | T | H | E | R | P | R | I | P | A |
| 927 | V | T | H | E | R | P | R | I | S | S |
| 928 | V | T | H | E | R | P | R | I | S | A |
| 929 | V | T | H | A | C | S | Q | V | P | S |
| 930 | V | T | H | A | C | S | Q | V | P | A |
| 931 | V | T | H | A | C | S | Q | V | S | S |
| 932 | V | T | H | A | C | S | Q | V | S | A |
| 933 | V | T | H | A | C | S | Q | I | P | S |
| 934 | V | T | H | A | C | S | Q | I | P | A |
| 935 | V | T | H | A | C | S | Q | I | S | S |
| 936 | V | T | H | A | C | S | Q | I | S | A |
| 937 | V | T | H | A | C | S | R | V | P | S |
| 938 | V | T | H | A | C | S | R | V | P | A |
| 939 | V | T | H | A | C | S | R | V | S | S |
| 940 | V | T | H | A | C | S | R | V | S | A |
| 941 | V | T | H | A | C | S | R | I | P | S |
| 942 | V | T | H | A | C | S | R | I | P | A |
| 943 | V | T | H | A | C | S | R | I | S | S |
| 944 | V | T | H | A | C | S | R | I | S | A |
| 945 | V | T | H | A | C | P | Q | V | P | S |
| 946 | V | T | H | A | C | P | Q | V | P | A |
| 947 | V | T | H | A | C | P | Q | V | S | S |
| 948 | V | T | H | A | C | P | Q | V | S | A |
| 949 | V | T | H | A | C | P | Q | I | P | S |
| 950 | V | T | H | A | C | P | Q | I | P | A |
| 951 | V | T | H | A | C | P | Q | I | S | S |
| 952 | V | T | H | A | C | P | Q | I | S | A |
| 953 | V | T | H | A | C | P | R | V | P | S |
| 954 | V | T | H | A | C | P | R | V | P | A |
| 955 | V | T | H | A | C | P | R | V | S | S |
| 956 | V | T | H | A | C | P | R | V | S | A |
| 957 | V | T | H | A | C | P | R | I | P | S |
| 958 | V | T | H | A | C | P | R | I | P | A |
| 959 | V | T | H | A | C | P | R | I | S | S |
| 960 | V | T | H | A | C | P | R | I | S | A |
| 961 | V | T | H | A | R | S | Q | V | P | S |
| 962 | V | T | H | A | R | S | Q | V | P | A |
| 963 | V | T | H | A | R | S | Q | V | S | S |
| 964 | V | T | H | A | R | S | Q | V | S | A |
| 965 | V | T | H | A | R | S | Q | I | P | S |
| 966 | V | T | H | A | R | S | Q | I | P | A |
| 967 | V | T | H | A | R | S | Q | I | S | S |
| 968 | V | T | H | A | R | S | Q | I | S | A |
| 969 | V | T | H | A | R | S | R | V | P | S |
| 970 | V | T | H | A | R | S | R | V | P | A |
| 971 | V | T | H | A | R | 3 | R | V | S | S |
| 972 | V | T | H | A | R | S | R | V | S | A |
| 973 | V | T | H | A | R | S | R | I | P | S |
| 974 | V | T | H | A | R | S | R | I | P | A |
| 975 | V | T | H | A | R | S | R | I | S | S |
| 976 | V | T | H | A | R | S | R | I | S | A |
| 977 | V | T | H | A | R | P | Q | V | P | S |
| 978 | V | T | H | A | R | P | Q | V | P | A |
| 979 | V | T | H | A | R | P | Q | V | S | S |
| 980 | V | T | H | A | R | P | Q | V | S | A |
| 981 | V | T | H | A | R | P | Q | I | P | S |
| 982 | V | T | H | A | R | P | Q | I | P | A |
| 983 | V | T | H | A | R | P | Q | I | S | S |
| 984 | V | T | H | A | R | P | Q | I | S | A |
| 985 | V | T | H | A | R | P | R | V | P | S |
| 986 | V | T | H | A | R | P | R | V | P | A |
| 987 | V | T | H | A | R | P | R | V | S | S |
| 988 | V | T | H | A | R | P | R | V | S | A |
| 989 | V | T | H | A | R | P | R | I | P | S |
| 990 | V | T | H | A | R | P | R | I | P | A |
| 991 | V | T | H | A | R | P | R | I | S | S |
| 992 | V | T | H | A | R | P | R | I | S | A |

The invention also includes IL4Rα peptide variants, which are any fragments of an IL4Rα protein variant that contains one or more of the amino acid variations shown in Table 2. An IL4Rα peptide variant is at least 6 amino acids in length and is preferably any number between 6 and 30 amino acids long, more preferably between 10 and 25, and most preferably between 15 and 20 amino acids long. Such IL4Rα peptide variants may be useful as antigens to generate antibodies specific for one of the above IL4Rα isoforms. In addition, the IL4Rα peptide variants may be useful in drug screening

An IL4Rα variant protein or peptide of the invention may be prepared by chemical synthesis or by expressing one of the variant IL4Rα genomic and cDNA sequences as described above. Alternatively, the IL4Rα protein variant may be isolated from a biological sample of an individual having an IL4Rα isogene which encodes the variant protein. Where the sample contains two different IL4Rα isoforms (i.e., the individual has different IL4Rα isogenes), a particular IL4Rα isoform of the invention can be isolated by immunoaffinity chromatography using an antibody which specifically binds to that particular IL4Rα isoform but does not bind to the other IL4Rα isoform.

The expressed or isolated IL4Rα protein may be detected by methods known in the art, including Coomassie blue staining, silver staining, and Western blot analysis using antibodies specific for the isoform of the IL4Rα protein as discussed further below. IL4Rα variant proteins can be purified by standard protein purification procedures known in the art, including differential precipitation, molecular sieve chromatography, ion-exchange chromatography, isoelectric focusing, gel electrophoresis, affinity and immunoaffinity chromatography and the like. (Ausubel et. al., 1987, In Current Protocols in Molecular Biology John Wiley and Sons, New York, N.Y.). In the case of immunoaffinity chromatography, antibodies specific for a particular polymorphic variant may be used.

A polymorphic variant IL4Rα gene of the invention may also be fused in frame with a heterologous sequence to encode a chimeric IL4Rα protein. The non-IL4Rα portion of the chimeric protein may be recognized by a commercially available antibody. In addition, the chimeric protein may also be engineered to contain a cleavage site located between the IL4Rα and non-IL4Rα portions so that the IL4Rα protein may be cleaved and purified away from the non-IL4Rα portion.

An additional embodiment of the invention relates to using a novel IL4Rα protein isoform in any of a variety of drug screening assays. Such screening assays may be performed to identify agents that bind specifically to all known IL4Rα protein isoforms or to only a subset of one or more of these isoforms. The agents may be from chemical compound libraries, peptide libraries and the like. The IL4Rα protein or peptide variant may be free in solution or affixed to a solid support. In one embodiment, high throughput screening of compounds for binding to an IL4Rα variant may be accomplished using the method described in PCT application WO84/03565, in which large numbers of test compounds are synthesized on a solid substrate, such as plastic pins or some other surface, contacted with the IL4Rα protein(s) of interest and then washed. Bound IL4Rα protein(s) are then detected using methods well-known in the art.

In another embodiment, a novel IL4Rα protein isoform may be used in assays to measure the binding affinities of one or more candidate drugs targeting the IL4Rα protein.

In another embodiment, the invention provides antibodies specific for and immunoreactive with one or more of the novel IL4Rα variant proteins described herein. The antibodies may be either monoclonal or polyclonal in origin. The IL4Rα protein or peptide variant used to generate the antibodies may be from natural or recombinant sources or produced by chemical synthesis using synthesis techniques known in the art. If the IL4Rα protein variant is of insufficient size to be antigenic, it may be conjugated, complexed, or otherwise covalently linked to a carrier molecule to enhance the antigenicity of the peptide. Examples of carrier molecules, include, but are not limited to, albumins (e.g., human, bovine, fish, ovine), and keyhole limpet hemocyanin (Basic and Clinical Immunology, 1991, Eds. D. P. Stites, and A. I. Terr, Appleton and Lange, Norwalk Conn., San Mateo, Calif.).

In one embodiment, an antibody specifically immunoreactive with one of the novel IL4Rα protein isoforms described herein is administered to an individual to neutralize activity of the IL4Rα isoform expressed by that individual. The antibody may be formulated as a pharmaceutical composition which includes a pharmaceutically acceptable carrier.

Antibodies specific for and immunoreactive with one of the novel IL4Rα protein isoform described herein may be used to immunoprecipitate the IL4Rα protein variant from solution as well as react with IL4Rα protein isoforms on Western or immunoblots of polyacrylamide gels on membrane supports or substrates. In another preferred embodiment, the antibodies will detect IL4Rα protein isoforms in paraffin or frozen tissue sections, or in cells which have been fixed or unfixed and prepared on slides, coverslips, or the like, for use in immunocytochemical, immunohistochemical, and immunofluorescence techniques.

In another embodiment, an antibody specifically immunoreactive with one of the novel IL4Rα protein variants described herein is used in immunoassays to detect this variant in biological samples. In this method, an antibody of the present invention is contacted with a biological sample and the formation of a complex between the IL4Rα protein variant and the antibody is detected. As described, suitable immunoassays include radioimmunoassay, Western blot assay, immunofluorescent assay, enzyme linked immunoassay (ELISA), chemiluminescent assay, immunohistochemical assay, immunocytochemical assay, and the like (see, e.g., Principles and Practice of Immunoassay, 1991, Eds. Christopher P. Price and David J. Neoman, Stockton Press, New York, N.Y.; Current Protocols in Molecular Biology, 1987, Eds. Ausubel et al., John Wiley and Sons, New York, N.Y.). Standard techniques known in the art for ELISA are described in Methods in Immunodiagnosis, 2nd Ed., Eds. Rose and Bigazzi, John Wiley and Sons, New York 1980; and Campbell et al., 1984, Methods in Immunology, W. A. Benjamin, Inc.). Such assays may be direct, indirect, competitive, or noncompetitive as described in the art (see, e.g., Principles and Practice of Immunoassay, 1991, Eds. Christopher P. Price and David J. Neoman, Stockton Pres, NY, N.Y.; and Oellirich, M., 1984, J. Clin. Chem. Clin. Biochem., 22:895-904). Proteins may be isolated from test specimens and biological samples by conventional methods, as described in Current Protocols in Molecular Biology, supra.

Exemplary antibody molecules for use in the detection and therapy methods of the present invention are intact immunoglobulin molecules, substantially intact inununoglobulin molecules, or those portions of immunoglobulin molecules that contain the antigen binding site. Polyclonal or monoclonal antibodies may be produced by methods conventionally known in the art (e.g., Kohler and Milstein, 1975, Nature, 256:495-497; Campbell Monoclonal Antibody Technology, the Production and Characterization of Rodent and Human Hybridomas, 1985, In: Laboratory Techniques in Biochemistry and Molecular Biology, Eds. Burdon et al., Volume 13, Elsevier Science Publishers, Amsterdam). The antibodies or antigen binding fragments thereof may also be produced by genetic engineering. The technology for expression of both heavy and light chain genes in E. coli is the subject of PCT patent applications, publication number WO 901443, WO 901443 and WO 9014424 and in Huse et al., 1989, Science, 246:1275-1281. The antibodies may also be humanized (e.g., Queen, C. et al. 1989 Proc. Natl. Acad. Sci. 86;10029).

Effect(s) of the polymorphisms identified herein on expression of IL4Rα may be investigated by preparing recombinant cells and/or organisms, preferably recombinant animals, containing a polymorphic variant of the IL4Rα gene. As used herein, “expression” includes but is not limited to one or more of the following: transcription of the gene into precursor mRNA; splicing and other processing of the precursor mRNA to produce mature mRNA; mRNA stability; translation of the mature mRNA into IL4Rα protein (including codon usage and tRNA availability); and glycosylation and/or other modifications of the translation product, if required for proper expression and function.

To prepare a recombinant cell of the invention, the desired IL4Rα isogene may be introduced into the cell in a vector such that the isogene remains extrachromosomal. In such a situation, the gene will be expressed by the cell from the extrachromosomal location. In a preferred embodiment, the IL4Rα isogene is introduced into a cell in such a way that it recombines with the endogenous IL4Rα gene present in the cell. Such recombination requires the occurrence of a double recombination event, thereby resulting in the desired IL4Rα gene polymorphism. Vectors for the introduction of genes both for recombination and for extrachromosomal maintenance are known in the art, and any suitable vector or vector construct may be used in the invention. Methods such as electroporation, particle bombardment, calcium phosphate co-precipitation and viral transduction for introducing DNA into cells are known in the art; therefore, the choice of method may lie with the competence and preference of the skilled practitioner. Examples of cells into which the IL4Rα isogene may be introduced include, but are not limited to, continuous culture cells, such as COS, NIH/3T3, and primary or culture cells of the relevant tissue type, i.e., they express the IL4Rα isogene. Such recombinant cells can be used to compare the biological activities of the different protein variants.

Recombinant organisms, i.e., transgenic animals, expressing a variant IL4Rα gene are prepared using standard procedures known in the art. Preferably, a construct comprising the variant gene is introduced into a nonhuman animal or an ancestor of the animal at an embryonic stage, i.e., the one-cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes of interest, and other components known to those skilled in the art to provide a complete shuttle vector harboring the insulated gene(s) as a transgene, see e.g., U.S. Pat. No. 5,610,053. Another method involves directly injecting a transgene into the embryo. A third method involves the use of embryonic stem cells. Examples of animals into which the IL4Rα isogenes may be introduced include, but are not limited to, mice, rats, other rodents, and nonhuman primates (see “The Introduction of Foreign Genes into Mice” and the cited references therein, In: Recombinant DNA, Eds. J. D. Watson, M. Gilman, J. Witkowski, and M. Zoller; W. H. Freeman and Company, New York, pages 254-272). Transgenic animals stably expressing a human IL4Rα isogene and producing human IL4Rα protein can be used as biological models for studying diseases related to abnormal IL4Rα expression and/or activity, and for screening and assaying various candidate drugs, compounds, and treatment regimens to reduce the symptoms or effects of these diseases.

An additional embodiment of the invention relates to pharmaceutical compositions for treating disorders affected by expression or function of a novel IL4Rα isogene described herein. The pharmaceutical composition may comprise any of the following active ingredients: a polynucleotide comprising one of these novel IL4Rα isogenes; an antisense oligonucleotide directed against one of the novel IL4Rα isogenes, a polynucleotide encoding such an antisense oligonucleotide, or another compound which inhibits expression of a novel IL4Rα isogene described herein. Preferably, the composition contains the active ingredient in a therapeutically effective amount. By therapeutically effective amount is meant that one or more of the symptoms relating to disorders affected by expression or function of a novel IL4Rα isogene is reduced and/or eliminated. The composition also comprises a pharmaceutically acceptable carrier, examples of which include, but are not limited to, saline, buffered saline, dextrose, and water. Those skilled in the art may employ a formulation most suitable for the active ingredient, whether it is a polynucleotide, oligonucleotide, protein, peptide or small molecule antagonist. The pharmaceutical composition may be administered alone or in combination with at least one other agent, such as a stabilizing compound. Administration of the pharmaceutical composition may be by any number of routes including, but not limited to oral, intravenous, intramuscular, intra-arterial, intramedullary, intrathecal, intraventricular, intradermal, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.).

For any composition, determination of the therapeutically effective dose of active ingredient and/or the appropriate route of administration is well within the capability of those skilled in the art. For example, the dose can be estimated initially either in cell culture assays or in animal models. The animal model may also be used to determine the appropriate concentration range and route of administration. Such information can then be used to determine useful doses and routes for administration in humans. The exact dosage will be determined by the practitioner, in light of factors relating to the patient requiring treatment, including but not limited to severity of the disease state, general health, age, weight and gender of the patient, diet, time and frequency of administration, other drugs being taken by the patient, and tolerance/response to the treatment.

Information on the identity of genotypes and haplotypes for the IL4Rα gene of any particular individual as well as the frequency of such genotypes and haplotypes in any particular population of individuals is expected to be useful for a variety of basic research and clinical applications. Thus, the invention also provides compositions and methods for detecting the novel IL4Rα polymorphisms identified herein.

The compositions comprise at least one IL4Rα genotyping oligonucleotide. In one embodiment, an IL4Rα genotyping oligonucleotide is a probe or primer capable of hybridizing to a target region that is located close to, or that contains, one of the novel polymorphic sites described herein. As used herein, the term “oligonucleotide” refers to a polynucleotide molecule having less than about 100 nucleotides. A preferred oligonucleotide of the invention is 10 to 35 nucleotides long. More preferably, the oligonucleotide is between 15 and 30, and most preferably, between 20 and 25 nucleotides in length. The oligonucleotide may be comprised of any phosphorylation state of ribonucleotides, deoxyribonucleotides, and acyclic nucleotide derivatives, and other functionally equivalent derivatives. Alternatively, oligonucleotides may have a phosphate-free backbone, which may be comprised of linkages such as carboxymethyl, acetamidate, carbamate, polyamide (peptide nucleic acid (PNA)) and the like (Varma, R. in Molecular Biology and Biotechnology, A Comprehensive Desk Reference, Ed. R. Meyers, VCH Publishers, Inc. (1995), pages 617-620). Oligonucleotides of the invention may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Genotyping oligonucleotides of the invention must be capable of specifically hybridizing to a target region of an IL4Rα polynucleotide, i.e., an IL4Rα isogene. As used herein, specific hybridization means the oligonucleotide forms an anti-parallel double-stranded structure with the target region under certain hybridizing conditions, while failing to form such a structure when incubated with a non-target region or a non-IL4Rα polynucleotide under the same hybridizing conditions. Preferably, the oligonucleotide specifically hybridizes to the target region under conventional high stringency conditions. The skilled artisan can readily design and test oligonucleotide probes and primers suitable for detecting polymorphisms in the IL4Rα gene using the polymorphism information provided herein in conjunction with the known sequence information for the IL4Rα gene and routine techniques.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, by Sambrook J. et al., in Molecular Cloning, A Laboratory Manual, 2^{nd} Edition, Cold Spring Harbor Press, Cold Spring Harbor, NY (1989) and by Haymes, B. D. et al. in Nucleic Acid Hybridization, A Practical Approach, IRL Press, Washington, D.C. (1985). While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the oligonucleotide probe or primer as long as the resulting probe or primer is still capable of specifically hybridizing to the target region.

Preferred genotyping oligonucleotides of the invention are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a polymorphic site while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR Protocols, A Guide to Methods and Applications, Academic Press, 1990 and Ruano et al., 87 Proc. Natl. Acad. Sci. USA 6296-6300, 1990. Typically, an allele-specific oligonucleotide will be perfectly complementary to one allele while containing a single mismatch for another allele.

Allele-specific oligonucleotide probes which usually provide good discrimination between different alleles are those in which a central position of the oligonucleotide probe aligns with the polymorphic site in the target region (e.g., approximately the 7^{th} or 8^{th} position in a 15 mer, the 8^{th} or 9^{th} position in a 16mer, the 10^{th} or 11 ^{th} position in a 20 mer). A preferred ASO probe for detecting IL4Rα gene polymorphisms comprises a nucleotide sequence, listed 5′ to 3′, selected from the group consisting of:

| | | |
|---|---|---|
| TTGCACCACTGCACT | and its complement, | (SEQ ID NO:4) |
| TTGCACCGCTGCACT | and its complement, | (SEQ ID NO:5) |
| TTTTGTGCTATTCCC | and its complement, | (SEQ ID NO:6) |
| TTTTGTGTTATTCCC | and its complement, | (SEQ ID NO:7) |
| CTGGGCCGCTCAGGC | and its complement, | (SEQ ID NO:8) |
| CTGGGCCTCTCAGGC | and its complement, | (SEQ ID NO:9) |
| TAAGCCTGCGCTGGA | and its complement, | (SEQ ID NO:10) |
| TAAGCCTCCGCTGGA | and its complement, | (SEQ ID NO:11) |
| AGAACAACGGAGGCG | and its complement, | (SEQ ID NO:12) |
| AGAACAATGGAGGCG | and its complement, | (SEQ ID NO:13) |
| CGGAGGCGCGGGGTG | and its complement, | (SEQ ID NO:14) |
| CGGAGGCACGGGGTG | and its complement, | (SEQ ID NO:15) |
| GTGCGGATAACTATA | and its complement, | (SEQ ID NO:16) |
| GTGCGGACAACTATA | and its complement, | (SEQ ID NO:17) |
| CGGAGTGCGGCAGGG | and its complement, | (SEQ ID NO:18) |
| CGGAGTGTGGCAGGG | and its complement, | (SEQ ID NO:19) |
| GCCTGGGCTGAGGGT | and its complement, | (SEQ ID NO:20) |
| GCCTGGGTTGAGGGT | and its complement, | (SEQ ID NO:21) |
| TGGGGTGGGCAGGGG | and its complement, | (SEQ ID NO:22) |
| TGGGGTGAGCAGGGG | and its complement, | (SEQ ID NO:23) |
| TTCTCCCGCAGTGAA | and its complement, | (SEQ ID NO:24) |
| TTCTCCCACAGTGAA | and its complement, | (SEQ ID NO:25) |
| GTGAAAACGACCCGG | and its complement, | (SEQ ID NO:26) |
| GTGAAAATGACCCGG | and its complement, | (SEQ ID ND:27) |
| GGCAAGCCCTGGGGC | and its complement, | (SEQ ID NO:28) |
| GGCAAGCTCTGGGGC | and its complement, | (SEQ ID NO:29) |
| GCCCTGGGGCTGGAT | and its complement, | (SEQ ID NO:30) |
| GCCCTGGAGCTGGAT | and its complement, | (SEQ ID NO:31) |
| ATAGCAAATCCCAGG | and its complement, | (SEQ ID NO:32) |
| ATAGCAATTCCCAGG | and its complement, | (SEQ ID NO:33) |
| GCTCTGCCCTAGGCA | and its complement, | (SEQ ID NO:34) |
| GCTCTGCACTAGGCA | and its complement, | (SEQ ID NO:35) |
| CCCCCACCCCTCACA | and its complement, | (SEQ ID NO:36) |
| CCCOCACTCCTCACA | and its complement, | (SEQ ID NO:37) |
| TCCCTCCGCATCGCA | and its complement, | (SEQ ID NO:38) |
| TCCCTCCACATCGCA | and its complement, | (SEQ ID NO:39) |
| CACCTGCTGTGGTGT | and its complement, | (SEQ ID NO:40) |
| CACCTGCCGTGGTGT | and its complement, | (SEQ ID NO:41) |
| ATGTCTGAAGTAGAC | and its complement, | (SEQ ID NO:42) |
| ATGTCTGCAGTAGAC | and its complement, | (SEQ ID NO:43) |
| TGACCAACCTTTGCT | and its complement, | (SEQ ID NO:44) |
| TGACCAACTTTTGCT | and its complement, | (SEQ ID NO:45) |
| CCTGTTTTCTGGAGC | and its complement, | (SEQ ID NO:46) |
| CCTGTTTCCTGGAGC | and its complement, | (SEQ ID NO:47) |
| TGGAGCTGCTCGGAG | and its complement, | (SEQ ID NO:48) |
| TGGACCTTCTCGGAG | and its complement, | (SEQ ID NO:49) |
| AGTCATGCCTTCTTC | and its complement, | (SEQ ID NO:50) |
| AGTCATGTCTTCTTC | and its complement, | (SEQ ID NO:51) |
| GCCTTCTTCCACCTT | and its complement, | (SEQ ID NO:52) |
| GCCTTCTCCCACCTT | and its complement, | (SEQ ID NO:53) |
| CAGCCCCCGTCTCGG | and its complement, | (SEQ ID NO:54) |
| CAGCCCCTGTCTCGG | and its complement, | (SEQ ID NO:55) |
| GGAGTTTGTACATGC | and its complement, | (SEQ ID NO:56) |
| GGAGTTTATACATGC | and its complement, | (SEQ ID NO:57) |
| CAGCTCCCCAGAGCA | and its complement, | (SEQ ID NO:58) |
| CAGCTCCTCAGAGCA | and its complement, | (SEQ ID NO:59) |
| AGACAGGTCCTCGCC | and its complement, | (SEQ ID NO:60) |
| AGACAGGGCCTCGCC | and its complement, | (SEQ ID NO:61) |
| CTGCCCCTGGCAATG | and its complement, | (SEQ ID NO:62) |
| CTGCCCCCGGCAATG | and its complement, | (SEQ ID NO:63) |
| AGGTGCATGTCCTCT | and its complement, | (SEQ ID NO:64) |
| AGGTGCACGTCCTCT | and its complement, | (SEQ ID NO:65) |
| GTGCATGTCCTCTTG | and its complement, | (SEQ ID NO:66) |
| GTGCATGCCCTCTTG | and its complement, | (SEQ ID NO:67) |
| GGCTTATCCATGCCT | and its complement, | (SEQ ID NO:68) |
| GGCTTATTCATGCCT | and its complement, | (SEQ ID NO:69) |
| AGCCAGGCTGGCAGA | and its complement, | (SEQ ID NO:70) |
| AGCCAGGGTGGCAGA | and its complement, | (SEQ ID NO:71) |
| GGCCGACATGGAGGC | and its complement, | (SEQ ID NO:72) |
| GGCCCACGTGGAGGC | and its complement, | (SEQ ID NO:73) |
| TAACACAGCCATCAA | and its complement, | (SEQ ID NO:74) |
| TAACACAACCATCAA | and its complement, | (SEQ ID NO:75) |
| TAATGCTCGTCTGTG | and its complement, | (SEQ ID NO:76) |
| TAATGCTTGTCTGTG | and its complement, | (SEQ ID NO:77) |
| ACTTGCCGTCTGGGT | and its complement, | (SEQ ID NO:78) |
| and | | |
| ACTTGCCATCTGGGT | and its complement. | (SEQ ID NO:79) |

An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide primers hybridizing to either the coding or noncoding strand are contemplated by the invention. A preferred ASO primer for detecting IL4Rα gene polymorphisms comprises a nucleotide sequence, listed 5′ to 3′, selected from the group consisting of:

| | | | |
|---|---|---|---|
| CTGAGATTGCACCAC; | (SEQ ID NO:80) | GGCTGGAGTGCAGTG; | (SEQ ID NO:81) |
| CTGAGATTGCACCGC; | (SEQ ID NO:82) | GCCTGGAGTGCAGCG; | (SEQ ID NO:83) |
| CTGTGCTTTTGTGCT; | (SEQ ID NO:84) | ACCAAGGGGAATAGC; | (SEQ ID NO:85) |
| CTGTGCTTTTGTGTT; | (SEQ ID NO:86) | ACCAAGGGGAATAAC; | (SEQ ID NO:87) |
| GAGTTCCTGGGCCGC; | (SEQ ID NO:88) | GGAGCAGCCTGAGCG; | (SEQ ID NO:89) |
| GAGTTCCTGGGCCTC; | (SEQ ID NO:90) | GGAGCAGCCTGAGAG; | (SEQ ID NO:91) |
| TCCGAGTAAGCCTGC; | (SEQ ID NO:92) | TCCAGCTCCAGCGCA; | (SEQ ID NO:93) |
| TCCGAGTAAGCCTCC; | (SEQ ID NO:94) | TCCAGCTCCAGCGGA; | (SEQ ID NO:95) |
| TCCCTGAGAACAACG; | (SEQ ID NO:96) | ACCCCGCGCCTCCGT; | (SEQ ID NO:97) |
| TCCCTGAGAACAATG; | (SEQ ID NO:98) | ACCCCGCGCCTCCAT; | (SEQ ID NQ:99) |
| GAACAACGGAGGCGC; | (SEQ ID NO:100) | CACACGCACCCCGCG; | (SEQ ID NO:101) |
| GAACAACGGAGGCAC; | (SEQ ID NO:102) | CACACGCACCCCGTG; | (SEQ ID NO:103) |
| TGGTCAGTGCGGATA; | (SEQ ID NO:104) | CCAGTGTATAGTTAT; | (SEQ ID NO:105) |
| TGGTCAGTGCGGACA; | (SEQ ID NO:106) | CCAGTGTATAGTTGT; | (SEQ TD NO:107) |
| GCAGGGCGGAGTGCG; | (SEQ ID NO:108) | AGCCACCCCTGCCGC; | (SEQ ID NO:109) |
| GCAGGGCGGAGTGTG; | (SEQ ID NO:110) | AGCCACCCCTGCCAC; | (SEQ ID NO:111) |
| ACAGCTGCCTGGGCT; | (SEQ ID NO:112) | CACCGCACCCTCAGC; | (SEQ ID NO:113) |
| ACAGCTGCCTGGGTT; | (SEQ ID NO:114) | CACCCCACCCTCAAC; | (SEQ ID NO:115) |
| TGAGGGTGGGGTGGG; | (SEQ ID NO:116) | CCTCCTCCCCTGCCC; | (SEQ ID NO:117) |
| TGAGGGTGGGGTGAG; | (SEQ ID NO:118) | CCTCCTCCCCTGCTC; | (SEQ ID NO:119) |
| GGCCGCTTCTCCCGC; | (SEQ ID NO:120) | CTGGGTTTCACTGCG; | (SEQ ID NO:121) |
| GGCCGCTTCTCCCAC; | (SEQ ID NO:122) | CTGGGTTTCACTGTG; | (SEQ ID NO:123) |
| TTTGGAGTGAAAACG; | (SEQ ID NO:124) | CATCTGCCGGGTCGT; | (SEQ ID NO:125) |
| TTTGGAGTGAAAATG; | (SEQ ID NO:126) | CATCTGCCGGGTCAT; | (SEQ ID NO:127) |
| CTGGGAGGCAAGCCC; | (SEQ ID NO:128) | TATCCAGCCCCAGGG; | (SEQ ID NO:129) |
| CTGGGAGGCAAGCTC; | (SEQ ID NO:130) | TATCCAGCCOCAGAG; | (SEQ ID NO:131) |
| AGGCAAGCCCTGGGG; | (SEQ ID NO:132) | TTTGCTATCCAGCCC; | (SEQ ID NO:133) |
| AGGCAAGCCCTGGAG; | (SEQ ID NO:134) | TTTGCTATCCAGCTC; | (SEQ ID NO:135) |
| GGCTGGATAGCAAAT; | (SEQ ID NO:136) | CTAGCTCCTGGGATT; | (SEQ ID NO:137) |
| GGCTGGATAGCAATT; | (SEQ ID NQ:138) | CTAGCTCCTGGGAAT; | (SEQ ID NO:139) |
| CACCTGGCTCTGCCC; | (SEQ ID NO:140) | GGGACTTGCCTAGGG; | (SEQ ID NO:141) |
| CACCTGGCTCTGCAC; | (SEQ ID NO:142) | GGGACTTGCCTAGTG; | (SEQ ID NO:143) |
| CCTGGCCCCCCACCC; | (SEQ ID NO:144) | CTCTGATGTGAGGGG; | (SEQ ID NO:145) |
| CCTGGCCCCCCACTC; | (SEQ ID NO:146) | CTCTGATGTGAGGAG; | (SEQ ID NO:147) |
| GAACCCTCCCTCCGC; | (SEQ ID NO:148) | GCTGGCTGCGATGCG; | (SEQ ID NO:149) |
| GAACCCTCCCTCCAC; | (SEQ ID NO:150) | GCTGGCTGCGATGTG; | (SEQ ID NO:151) |
| TAGATACACCTGCTG; | (SEQ ID NO:152) | GCAGATACACCACAG; | (SEQ ID NO:153) |
| TAGATACACCTGCCG; | (SEQ ID NO:154) | GCAGATACACCACGG; | (SEQ ID NO:155) |
| GAAGGCATGTCTGAA; | (SEQ ID NO:156) | ATGGCTGTCTACTTC; | (SEQ ID NO:157) |
| GAAGGCATGTCTGCA; | (SEQ ID NO:158) | ATGGCTGTCTACTGC; | (SEQ ID NO:159) |
| GAACCCTGACCAACC; | (SEQ ID NO:160) | TGCAAAAGCAAAGGA; | (SEQ ID NO:161) |
| GAACCCTGACCAATC; | (SEQ ID NO:162) | TGCAAAAGCAAAGAA; | (SEQ ID NO:163) |
| TCTTGCCCTGTTTTC; | (SEQ ID NO:164) | TGTTGTGCTCCAGAA; | (SEQ ID NO:165) |
| TCTTGCCCTGTTTCC; | (SEQ ID NO:166) | TGTTGTGCTCCAGGA; | (SEQ ID NO:167) |
| TGTTCCTGGACCTGC; | (SEQ ID NO:168) | TCTCCTCTCCGAGCA; | (SEQ ID NO:169) |
| TGTTCCTGGACCTTC; | (SEQ ID NO:170) | TCTOCTCTCCGAGAA; | (SEQ ID NO:171) |
| TGGGGGAGTCATGCC; | (SEQ ID NO:172) | AAGGTGGAAGAAGGC; | (SEQ ID NO:173) |
| TGGGGGAGTCATGTC; | (SEQ ID NQ:174) | AAGGTGGAAGAAGAC; | (SEQ ID NO:175) |
| AGTCATGCCTTCTTC; | (SEQ ID NO:176) | TTCCCGAAGGTGGAA; | (SEQ ID NO:177) |
| AGTCATGCCTTCTCC; | (SEQ ID NO:178) | TTCCCGAAGGTGGGA; | (SEQ ID NO:179) |
| CAGCTGCAGCCCCCG; | (SEQ ID NO:180) | TGGGGGCCGAGACGG; | (SEQ ID NO:181) |
| CAGCTGCAGCCCCTG; | (SEQ ID NO:182) | TGGGGGCCGAGACAG; | (SEQ ID NO:183) |
| CTATCAGGAGTTTGT; | (SEQ ID NO:184) | TCCACCGCATGTACA; | (SEQ ID NQ:135) |
| CTATCAGGAGTTTAT; | (SEQ ID NO:186) | TCCACCGCATGTATA; | (SEQ ID NO:187) |
| CCCAAGCAGCTCCCC; | (SEQ ID NO:188) | CCCAGGTGCTCTGGG; | (SEQ ID NO:189) |
| CCCAAGCAGCTCCTC; | (SEQ ID NO:190) | CCCAGGTGCTCTGAG; | (SEQ ID NO:191) |
| CTGTGGAGACAGGTC; | (SEQ ID NO:192) | GTAGGGGGCGAGGAC; | (SEQ ID NO:193) |
| CTGTGGAGACAGGGC; | (SEQ ID NO:194) | GTAGGGGGCGAGGCC; | (SEQ ID NO:195) |
| TCCATCCTGCCCCTG; | (SEQ ID NO:196) | TCTGAGCATTGCCAG; | (SEQ ID NO:197) |
| TCCATCCTGCCCCCG; | (SEQ ID NO:198) | TCTGAGCATTGCCGG; | (SEQ ID NO:199) |
| TCTCTTAGGTGCATG; | (SEQ ID NO:200) | GCAACAAGAGGACAT; | (SEQ ID NO:201) |
| TCTCTTAGGTGCACG; | (SEQ ID NO:202) | GCAACAAGAGGACGT; | (SEQ ID NO:203) |
| TCTTAGGTGCATGTC; | (SEQ ID NO:204) | CAGCAACAAGAGGAC; | (SEQ ID NO:205) |
| TCTTAGGTGCATGCC; | (SEQ ID NO:206) | CAGCAACAAGAGGGC; | (SEQ ID NO:207) |
| GACTAGGGCTTATCC; | (SEQ ID NO:208) | TTTCCCAGGCATGGA; | (SEQ ID NO:209) |
| GACTAGGGCTTATTC; | (SEQ ID NO:210) | TTTCCCAGGCATGAA; | (SEQ ID NO:211) |
| GAAGGCAGCCAGGCT; | (SEQ ID NO:212) | TGGAAATCTGCCAGC; | (SEQ ID NO:213) |
| GAAGGCAGCCAGGGT; | (SEQ ID NO:214) | TGGAAATCTGCCACC; | (SEQ ID NO:215) |
| GATCATGGCCCACAT; | (SEQ ID NO:216) | AGGTGGGCCTCCATG; | (SEQ ID NO:217) |
| GATCATGGCCCACGT; | (SEQ ID NO:213) | AGGTGGGCCTCCACG; | (SEQ ID NO:219) |
| AGAAACTAACACAGC; | (SEQ ID NO:220) | ATTCCCTTGATGGCT; | (SEQ ID NO:221) |
| AGAAACTAACACAAC; | (SEQ ID NO:222) | ATTCCCTTGATGGTT; | (SEQ ID NO:223) |
| GTTGAGTAATGCTCG; | (SEQ ID NO:224) | AAAACACACAGACGA; | (SEQ ID NO:225) |
| GTTGAGTAATGCTTG; | (SEQ ID NO:226) | AAAACACACAGACAA; | (SEQ ID NO:227) |
| TAAGAAACTTGCCGT; | (SEQ ID NO:228) | ACCCAAACCCAGACG; | (SEQ ID NO:229) |
| TAAGAAACTTGCCAT; | (SEQ ID NO:230) | and ACCCAAACCCAGATG. | (SEQ ID NO:231) |

Other genotyping oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such genotyping oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site. A particularly preferred oligonucleotide primer for detecting IL4Rα gene polymorphisms by primer extension terminates in a nucleotide sequence, listed 5′ to 3′, selected from the group consisting of:

| | | | |
|---|---|---|---|
| AGATTGCACC; | (SEQ ID NO:232) | TGGAGTGCAG; | (SEQ ID NO:233) |
| TGCTTTTGTG; | (SEQ ID NO:234) | AAGGGGAATA; | (SEQ ID NO:235) |
| TTCCTGGGCC; | (SEQ ID NO:236) | GCAGCCTGAG; | (SEQ ID NO:237) |
| GAGTAAGCCT; | (SEQ ID NO:238) | AGCTCCAGCG; | (SEQ ID NO:239) |
| CTGAGAACAA; | (SEQ ID NO:240) | CCGCGCCTCC; | (SEQ ID NO:241) |
| CAACGGAGGC; | (SEQ ID NO:242) | ACGCACCCCG; | (SEQ ID NO:243) |
| TCAGTGCGGA; | (SEQ ID NO:244) | GTGTATAGTT; | (SEQ ID NO:245) |
| GGGCGGAGTG; | (SEQ ID NO:246) | CACCCCTGCC; | (SEQ ID NO:247) |
| GCTGCCTGGG; | (SEQ ID NO:248) | CCCACCCTCA; | (SEQ ID NO:249) |
| GGGTGGGGTG; | (SEQ ID NO:250) | CCTCCCCTGC; | (SEQ ID NO:251) |
| CGCTTCTCCC; | (SEQ ID NO:252) | GGTTTCACTG; | (SEQ ID NO:253) |
| GGAGTGGAAA; | (SEQ ID NO:254) | CTGCCGGGTC; | (SEQ ID NO:255) |
| GGAGGCAAGC; | (SEQ ID NO:256) | CCAGCCCCAG; | (SEQ ID NO:257) |
| CAAGCCCTGG; | (SEQ ID NO:258) | GCTATCCAGC; | (SEQ ID NO:259) |
| TGGATAGCAA; | (SEQ ID NO:260) | GCTCCTGGGA; | (SEQ ID NO:261) |
| CTGGCTCTGC; | (SEQ ID NO:262) | ACTTGCCTAG; | (SEQ ID NO:263) |
| GGCCCCCCAC; | (SEQ ID NO:264) | TGATGTGAGG; | (SEQ ID NO:265) |
| CCCTCCCTCC; | (SEQ ID NO:266) | GGCTGCGATG; | (SEQ ID NO:267) |
| ATACACCTGC; | (SEQ ID NO:268) | GATACACCAC; | (SEQ ID NO:269) |
| GGCATGTCTG; | (SEQ ID NO:270) | GCTGTCTACT; | (SEQ ID NO:271) |
| CCCTGACCAA; | (SEQ ID NO:272) | AAAAGCAAAG; | (SEQ ID NO:273) |
| TGCCCTGTTT; | (SEQ ID NO:274) | TGTGCTCCAG; | (SEQ ID NO:275) |
| TCCTGGACCT; | (SEQ ID NO:276) | CCTCTCCGAG; | (SEQ ID NO:277) |
| GGGAGTCATG; | (SEQ ID NO:278) | GTGGAAGAAG; | (SEQ ID NO:279) |
| CATGCCTTCT; | (SEQ ID NQ:280) | CCGAAGGTGG; | (SEQ ID NO:281) |
| CTGCAGCCCC; | (SEQ ID NO:232) | GGGCCGAGAC; | (SEQ ID NQ:283) |
| TCAGGAGTTT; | (SEQ ID NO:284) | ACCGCATGTA; | (SEQ ID NO:285) |
| AAGCAGCTCC; | (SEQ ID NO:286) | AGGTGCTCTG; | (SEQ ID NO:287) |
| TGGAGACAGG; | (SEQ ID NO:288) | GGGGGCGAGG; | (SEQ ID NO:289) |
| ATCCTGCCCC; | (SEQ ID NO:290) | GAGCATTGCO; | (SEQ ID NO:291) |
| CTTAGGTGCA; | (SEQ ID NO:292) | ACAAGAGGAC; | (SEQ ID NO:293) |
| TAGGTGCATG; | (SEQ ID NO:294) | CAACAAGAGG; | (SEQ ID NO:295) |
| TAGGGCTTAT; | (SEQ ID NO:296) | CCCAGGCATG; | (SEQ ID NO:297) |
| GGCAGCCAGG; | (SEQ ID NO:298) | AAATCTGCCA; | (SEQ ID NO:299) |
| CATGGCCCAC; | (SEQ ID N0:300) | TGGGCCTCCA; | (SEQ ID NO:301) |
| AACTAACACA; | (SEQ ID NO:302) | COCTTGATGG; | (SEQ ID NO:303) |
| GAGTAATGCT; | (SEQ ID NO:304) | ACACACAGAC; | (SEQ ID NO:305) |
| GAAACTTGCC; | (SEQ ID NO:306) | and CAAACCCAGA. | (SEQ ID NO:307) |

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

IL4Rα genotyping oligonucleotides of the invention may also be immobilized on or synthesized on a solid surface such as a microchip, bead, or glass slide (see, e.g., WO 98/20020 and WO 98/20019). Such immobilized genotyping oligonucleotides may be used in a variety of polymorphism detection assays, including but not limited to probe hybridization and polymerase extension assays. Immobilized IL4Rα genotyping oligonucleotides of the invention may comprise an ordered array of oligonucleotides designed to rapidly screen a DNA sample for polymorphisms in multiple genes at the same time.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

The above described oligonucleotide compositions and kits are useful in methods for genotyping and/or haplotyping the IL4Rα gene in an individual. As used herein, the terms “IL4Rα genotype” and “IL4Rα haplotype” mean the genotype or haplotype contains the nucleotide pair or nucleotide, respectively, that is present at one or more of the novel polymorphic sites described herein and may optionally also include the nucleotide pair or nucleotide present at one or more additional polymorphic sites in the IL4Rα gene. The additional polymorphic sites may be currently known polymorphic sites or sites that are subsequently discovered.

One embodiment of the genotyping method involves isolating from the individual a nucleic acid mixture comprising the two copies of the IL4Rα gene, or a fragment thereof, that are present in the individual, and determining the identity of the nucleotide pair at one or more of the polymorphic sites selected from PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45 in the two copies to assign an IL4Rα genotype to the individual. As will be readily understood by the skilled artisan, the tow “copies” of a gene in an individual may be the same allele or may be different alleles. In a preferred embodiment of the genotyping method, the identity of the nucleotide pair atone or more of the polymorphic sites selected from the group consisting of PS5, PS24, PS26, PS29, PS31, PS42, and PS43 is also determined. In a particularly preferred embodiment, the genotyping method comprises determining the identity of the nucleotide pair at each of PS1-45.

Typically, the nucleic acid mixture is isolated from a biological sample taken from the individual, such as a blood sample or tissue sample. Suitable tissue samples include whole blood, semen saliva, tears, urine, fecal material, sweat, buccal, skin and hair. The nucleic acid mixture may be comprised of genomic DNA, mRNA, or cDNA and, in the latter two cases, the biological sample must be obtained from an organ in which the IL4Rα gene is expressed. Furthermore it will be understood by the skilled artisan that mRNA or cDNA preparations would not be used to detect polymorphisms located in introns or in 5′ and 3′ nontranscribed regions. If an IL4Rα gene fragment is isolated, it must contain the polymorphic site(s) to be genotyped.

One embodiment of the haplotyping method comprises isolating from the individual a nucleic acid molecule containing only one of the two copies of the IL4Rα gene, or a fragment thereof, that is present in the individual and determining in that copy the identity of the nucleotide at one or more of the polymorphic sites PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45 in that copy to assign an IL4Rα haplotype to the individual. The nucleic acid may be isolated using any method capable of separating the two copies of the IL4Rα gene or fragment such as one of the methods described above for preparing IL4Rα isogenes, with targeted in vivo cloning being the preferred approach. As will be readily appreciated by those skilled in the art, any individual clone will only provide haplotype information on one of the two IL4Rα gene copies present in an individual. If haplotype information is desired for the individual's other copy, additional IL4Rα clones will need to be examined. Typically, at least five clones should be examined to have more than a 90% probability of haplotyping both copies of the IL4Rα gene in an individual. In some embodiments, the haplotyping method also comprises identifying the nucleotide atone or more of the polymorphic sites PS5, PS24, PS26, PS29, PS31, PS42, and PS43. In a particularly preferred embodiment, the nucleotide at each of PS1-45 is identified.

In a preferred embodiment, an IL4Rα haplotype pair is determined for an individual by identifying the phased sequence of nucleotides at one or more of the polymorphic sites selected from PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45 in each copy of the IL4Rα gene that is present in the individual. In a particularly preferred embodiment, the haplotyping method comprises identifying the phased sequence of nucleotides at each of PS1-45 in each copy of the IL4Rα gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the IL4Rα gene, or fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

In addition, the identity of the allele(s) present at any of the novel polymorphic sites described herein may be indirectly determined by genotyping a polymorphic site not disclosed herein that is in linkage disequilibrium with the polymorphic site that is of interest. Two sites are said to be in linkage disequilibrium if the presence of a particular variant at one site enhances the predictability of another variant at the second site (Stevens, JC 1999, Mol. Diag. 4: 309-17). Polymorphic sites in linkage disequilibrium with the presently disclosed polymorphic sites may be located in regions of the gene or in other genomic regions not examined herein. Genotyping of a polymorphic site in linkage disequilibrium with the novel polymorphic sites described herein may be performed by, but is not limited to, any of the above-mentioned methods for detecting the identity of the allele at a polymorphic site.

The target region(s) may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-193, 1991; WO90/01069), and oligonucleotide ligation assay (OLA) (Landegren et al., Science 241:1077-1080, 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., Proc. Natl. Acad. Sci. USA 89:392-396, 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype or haplotype for the IL4Rα gene of an individual may also be determined by hybridization of a nucleic sample containing one or both copies of the gene to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nucl. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524. Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruaño et al., Nucl. Acids Res. 17:8392, 1989; Ruaño et al., Nucl. Acids Res. 19, 6877-6882, 1991; WO 93/22456; Turki et al., J. Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

In another aspect of the invention, an individual's IL4Rα haplotype pair is predicted from its IL4Rα genotype using information on haplotype pairs known to exist in a reference population. In its broadest embodiment, the haplotyping prediction method comprises identifying an IL4Rα genotype for the individual at two or more polymorphic sites selected from PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, PS45, enumerating all possible haplotype pairs which are consistent with the genotype, accessing data containing IL4Rα haplotype pairs identified in a reference population, and assigning a haplotype pair to the individual that is consistent with the data. In one embodiment, the reference haplotype pairs include the IL4Rα haplotype pairs shown in Table 4.

Generally, the reference population should be composed of randomly-selected individuals representing the major ethnogeographic groups of the world. A preferred reference population for use in the methods of the present invention comprises an approximately equal number of individuals from Caucasian, African American, Asian and Hispanic-Latino population groups with the minimum number of each group being chosen based on how rare a haplotype one wants to be guaranteed to see. For example, if one wants to have a q% chance of not missing a haplotype that exists in the population at a p% frequency of occurring in the reference population, the number of individuals (n) who must be sampled is given by 2n=log(1-q)/log(1-p) where p and q are expressed as fractions. A preferred reference population allows the detection of any haplotype whose frequency is at least 10% with about 99% certainty and comprises about 20 unrelated individuals from each of the four population groups named above. A particularly preferred reference population includes a 3-generation family representing one or more of the four population groups to serve as controls for checking quality of haplotyping procedures.

In a preferred embodiment, the haplotype frequency data for each ethnogeographic group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3^{rd} Ed., 1997) postulates that the frequency of finding the haplotype pair H₁/H₂ is equal to p_{H-W} (H₁/H₂)=2p(H₁)p(H₂) if H₁≠H₂ and p_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), SMD, or allele-specific long-range PCR (Michalotos-Beloin et al., Nucleic Acids Res. 24:4841-4843, 1996).

In one embodiment of this method for predicting an IL4Rα haplotype pair, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., Nucleic Acids Res. 24:4841-4843, 1996).

The invention also provides a method for determining the frequency of an IL4Rα genotype or IL4Rα haplotype in a population. The method comprises determining the genotype or the haplotype pair for the IL4Rα gene that is present in each member of the population, wherein the genotype or haplotype comprises the nucleotide pair or nucleotide detected at one or more of the polymorphic sites PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45 in the IL4Rα gene; and calculating the frequency any particular genotype or haplotype is found in the population. The population may be a reference population, a family population, a same sex population, a population group, a trait population (e.g., a group of individuals exhibiting a trait of interest such as a medical condition or response to a therapeutic treatment).

In another aspect of the invention, frequency data for IL4Rα genotypes and/or haplotypes found in a reference population are used in a method for identifying an association between a trait and an IL4Rα genotype or an IL4Rα haplotype. The trait may be any detectable phenotype, including but not limited to susceptibility to a disease or response to a treatment. The method involves obtaining data on the frequency of the genotype(s) or haplotype(s) of interest in a reference population as well as in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above. In another embodiment, the frequency data for the reference and/or trait populations is obtained by accessing previously determined frequency data, which may be in written or electronic form. For example, the frequency data may be present in a database that is accessible by a computer. Once the frequency data is obtained, the frequencies of the genotype(s) or haplotype(s) of interest in the reference and trait populations are compared. In a preferred embodiment, the frequencies of all genotypes and/or haplotypes observed in the populations are compared. If a particular genotype or haplotype for the IL4Rα gene is more frequent in the trait population than in the reference population at a statistically significant amount, then the trait is predicted to be associated with that IL4Rα genotype or haplotype. Preferably, the IL4Rα genotype or haplotype being compared in the trait and reference populations is selected from the full-genotypes and full-haplotypes shown in Tables 4 and 5, respectively, or from sub-genotypes and sub-haplotypes derived from these genotypes and haplotypes.

In a preferred embodiment of the method, the trait of interest is a clinical response exhibited by a patient to some therapeutic treatment, for example, response to a drug targeting IL4Rα or response to a therapeutic treatment for a medical condition. As used herein, “medical condition” includes but is not limited to any condition or disease manifested as one or more physical and/or psychological symptoms for which treatment is desirable, and includes previously and newly identified diseases and other disorders. As used herein the term “clinical response” means any or all of the following: a quantitative measure of the response, no response, and adverse response (i.e., side effects).

In order to deduce a correlation between clinical response to a treatment and an IL4Rα genotype or haplotype, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials. As used herein, the term “clinical trial” means any research study designed to collect clinical data on responses to a particular treatment, and includes but is not limited to phase I, phase II and phase III clinical trials. Standard methods are used to define the patient population and to enroll subjects.

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the IL4Rα gene for each individual in the trial population is genotyped and/or haplotyped, which may be done before or after administering the treatment.

After both the clinical and polymorphism data have been obtained, correlations between individual response and IL4Rα genotype or haplotype content are created. Correlations may be produced in several ways. In one method, individuals are grouped by their IL4Rα genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the IL4Rα gene give the most significant contribution to the differences in phenotype. One regression model useful in the invention starts with a model of the form

r=r₀+S×d

where r is the response, r₀ is a constant called the “intercept”, S is the slope and d is the dose. To determine the dose, the most-common and least common nucleotides at the polymorphic site are first defined. Then, for each individual in the trial population, one calculates a “dose” as the number of least-common nucleotides the individual has at the polymorphic site of interest. This value can be 0 (homozygous for the least-common nucleotide), 1 (heterozygous), or 2 (homozygous for the most common nucleotide). An individual's “response” is the value of the clinical measurement. Standard linear regression methods are then used to fit all the individuals' doses and responses to a single model (see e.g., L. D. Fisher and G. vanBelle, supra, Ch 9). The outputs of the regression calculation are the intercept r₀, the slope S, and the variance (which measures how well the data fits this simple linear model). The Students t-test value and the level of significance can then be calculated for each of the polymorphic sites.

A second method for finding correlations between IL4Rα haplotype content and clinical responses uses predictive models based on error-minimizing optimization algorithms. One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial Intelligence”, 2^{nd} Edition (McGraw-Hill, New York, 1991, Ch. 18), standard gradient descent methods (Press et al., supra Ch. 10), or other global or local optimization approaches (see discussion in Judson, supra) could also be used. As an example, a genetic algorithm approach is described herein. This method searches for optimal parameters or weights in linear or Don-linear models connecting IL4Rα haplotype loci and clinical outcome. One model is of the form

$$C = {C}_{0} + {\sum }_{α} ( {\sum }_{t} {w}_{i , α} {R}_{i , α} + {\sum }_{i} {w}_{i , α}^{′} {L}_{i , α} ) \qquad [ 1 ]$$

where C is the measured clinical outcome, i goes over all polymorphic sites, α over all candidate genes, C₀, w_{i,α}and w′_{i,α} are variable weight values, R_{i,α} is equal to 1 if site i in gene α in the first haplotype takes on the most common nucleotide and −1 if it takes on the less common nucleotide. L_{i,α} is the same as R_{i,α} except for the second haplotype. The constant term C₀ and the weights w_{i,α} and w′_{i,α} are varied by the genetic algorithm during a search process that minimizes the error between the measured value of C and the value calculated from Equation 1. Models other than the one given in Equation 1 can be readily incorporated by those skilled in the art for analyzing the clinical and polymorphism data. The genetic algorithm is especially suited for searching not only over the space of weights in a particular model but also over the space of possible models (Judson, supra).

Correlations may also be analyzed using analysis of variation (ANOVA) techniques to determine how much of the variation in the clinical data is explained by different subsets of the polymorphic sites in the IL4Rα gene. ANOVA is used to test hypotheses about whether a response variable is caused by or correlated with one or more traits or variables that can be measured (Fisher and vanBelle, supra, Ch. 10). These traits or variables are called the independent variables. To carry out ANOVA, the independent variable(s) are measured and individuals are placed into groups based on their values for these variables. In this case, the independent variable(s) refers to the combination of polymorphisms present at a subset of the polymorphic sites, and thus, each group contains those individuals with a given genotype or haplotype pair. The variation in response within the groups and also the variation between groups is then measured. If the within-group response variation is large (people in a group have a wide range of responses) and the response variation between groups is small (the average responses for all groups are about the same) then it can be concluded that the independent variables used for the grouping are not causing or correlated with the response variable. For instance, if people are grouped by month of birth (which should have nothing to do with their response to a drug) the ANOVA calculation should show a low level of significance. However, if the response variation is larger between groups than within groups, the F-ratio (=“between groups” divided by “within groups”) is greater than one. Large values of the F-ratio indicate that the independent variable is causing or correlated with the response. The calculated F-ratio is preferably compared with the critical F-distribution value at whatever level of significance is of interest. If the F-ratio is greater than the Critical F-distribution value, then one may be confident that the individual's genotype or haplotype pair for this particular subset of polymorphic sites in the IL4Rα gene is at least partially responsible for, or is at least strongly correlated with the clinical response.

From the analyses described above, a mathematical model may be readily constructed by the skilled artisan that predicts clinical response as a function of IL4Rα genotype or haplotype content. Preferably, the model is validated in one or more follow-up clinical trials designed to test the model.

The identification of an association between a clinical response and a genotype or haplotype (or haplotype pair) for the IL4Rα gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the IL4Rα gene), a serological test, or a physical exam measurement. The only requirement is that there be a good correlation between the diagnostic test results and the underlying IL4Rα genotype or haplotype that is in turn correlated with the clinical response. In a preferred embodiment, this diagnostic method uses the predictive haplotyping method described above.

Any or all analytical and mathematical operations involved in practicing the methods of the present invention may be implemented by a computer. In addition, the computer may execute a program that generates views (or screens) displayed on a display device and with which the user can interact to view and analyze large amounts of information relating to the IL4Rα gene and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations). The IL4Rα polymorphism data described herein may be stored as part of a relational database (e.g., an instance of an Oracle database or a set of ASCII flat files). These polymorphism data may be stored on the computer's hard drive or may, for example, be stored on a CD ROM or on one or more other storage devices accessible by the computer. For example, the data may be stored on one or more databases in communication with the computer via a network.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLES

The Examples herein are meant to exemplify the various aspects of carrying out the invention and are not intended to limit the scope of the invention in any way. The Examples do not include detailed descriptions for conventional methods employed, such as in the performance of genomic DNA isolation, PCR and sequencing procedures. Such methods are well-known to those skilled in the art and are described in numerous publications, for example, Sambrook, Fritsch, and Maniatis, “Molecular Cloning: A Laboratory Manual”, ₂nd Edition, Cold Spring Harbor Laboratory Press, USA, (1989).

### Example 1A

This example illustrates examination of various regions of the IL4Rα gene for polymorphic sites using DNA from Index Repository IA.

Amplification of Target Regions

The following target regions of the IL4Rα gene were amplified using the PCR primer pairs listed below, with the sequences presented in the 5′ to 3′ direction and nucleotide positions shown for each region corresponding to the indicated GenBank Accession No.

Accession Number: AC004525

| | | |
|---|---|---|
| Fragment 1 | | |
| Forward Primer | | |
| 32801-32822 | CCACAGTCATCCCGACACTAGC | (SEQ ID NO:308) |
| Reverse Primer | | |
| Complement of 33355-33334 | TATTCCAGCCGTATCCATGTGC | (SEQ ID NO: 309) |
| PCR product 555 nt | | |
| Fragment 2 | | |
| Forward Primer | | |
| 35515-35536 | CCTTGGTGCATGTGGTAAGAGG | (SEQ ID NO:310) |
| Reverse Primer | | |
| Complement of 36068-36046 | TTTCAAAGGTGGGAGGACTGAGG | (SEQ ID NO:311) |
| PCR product 554 nt | | |
| Fragment 3 | | |
| Forward Primer | | |
| 37031-37050 | GCAGTGAGCTGGGATTGTGC | (SEQ ID NO:312) |
| Reverse Primer | | |
| Complement of 37701-37679 | AACTCCCCTTCTCTGATGTGAGG | (SEQ ID NO:313) |
| PCR product 671 nt | | |
| Fragment 4 | | |
| Forward Primer | | |
| 43240-43262 | TCACAGTTACAGAGGTGGCAAGC | (SEQ ID NO:314) |
| Reverse Primer | | |
| Complement of 43727-43706 | CTGCCTACCTGGCAGATACACC | (SEQ ID NO:315) |
| PCR product 488 nt | | |
| Fragment 5 | | |
| Forward Primer | | |
| 49553-49574 | AGCTGTCACTCCACCTCCTTGG | (SEQ ID NO:316) |
| Reverse Primer | | |
| Complement of 50036-50013 | AAAGCCTCTGGTCTGCTAATGACC | (SEQ ID NO:317) |
| PCR product 484 nt | | |
| Fragment 6 | | |
| Forward Primer | | |
| 51394-51415 | GGGAGGAGATTCAGAGCACTCC | (SEQ ID NO:318) |
| Reverse Primer | | |
| Complement of 51847-51826 | CAGTCCACGTTTCCAGAACACC | (SEQ ID NO:319) |
| PCR product 454 nt | | |
| Fragment 7 | | |
| Forward Primer | | |
| 52806-52826 | GGCTTGGGATAATGGTGTTTGC | (SEQ ID NO:320) |
| Reverse Primer | | |
| Complement of 53529-53507 | TACTTCCCGAAGGTGGAAGAAGG | (SEQ ID NO:321) |
| PCR product 724 nt | | |
| Fragment 8 | | |
| Forward Primer | | |
| 53242-53265 | CAGTGGAGATCAGCAAGACAGTCC | (SEQ ID NO:322) |
| Reverse Primer | | |
| Complement of 53807-53786 | GGGCATCTCGGGTTCTACTTCC | (SEQ ID NO:323) |
| PCR product 566 nt | | |
| Fragment 9 | | |
| Forward Primer | | |
| 53522-53544 | GGGAAGTACGAGTGCTCACATGC | (SEQ ID NO:324) |
| Reverse Primer | | |
| Complement of 54110-54088 | CTTTATACCCCTCTTCCCCACTGC | (SEQ ID NO:325) |
| PCR product 589 nt | | |
| Fragment 10 | | |
| Forward Primer | | |
| 53821-53841 | TCTCTGAGCCAACCACTGTGC | (SEQ ID NO:326) |
| Reverse Primer | | |
| Complement of 54359-54337 | GGCTGAGTAGACAATGCCACTGC | (SEQ ID NO:327) |
| PCR product 539 nt | | |
| Fragment 11 | | |
| Forward Primer | | |
| 54055-54076 | CTGTGTCCCCAGAGAAATGTGG | (SEQ ID NO:328) |
| Reverse Primer | | |
| Complement of 54717-54695 | GACTCAGCAACAAGAGGACATGC | (SEQ ID NO:329) |
| PCR product 663 nt | | |
| Fragment 12 | | |
| Forward Primer | | |
| 54342-54365 | GGCATTGTCTACTCAGCCCTTACC | (SEQ ID NO:330) |
| Reverse Primer | | |
| Complement of 54986-54967 | ACAAGTCGAGGTGCCCAAGG | (SEQ ID NO:331) |
| PCR product 645 nt | | |
| Fragment 13 | | |
| Forward Primer | | |
| 54669-54693 | CCCACATACATGAGGGTCTCTTAGG | (SEQ ID NO:332) |
| Reverse Primer | | |
| Complement of 55270-55250 | ATTCTGCCTCCAGCATCAACC | (SEQ ID NO:333) |
| PCR product 602 nt | | |
| Fragment 14 | | |
| Forward Primer | | |
| 55235-55258 | AACAGAGCTTTCCTTTAGGTTGATGC | (SEQ ID NO:334) |
| Reverse Primer | | |
| Complement of 55847-55825 | CCTCAGTTCCCCACTACCTTAGC | (SEQ ID NO:335) |
| PCR product 613 nt | | |

These primer pairs were used in PCR reactions containing genomic DNA isolated from immortalized cell lines for each member of Index Repository IA. The PCR reactions were carried out under the following conditions:

| | |
|---|---|
| Reaction volume | = 20 μl |
| 10 × Advantage 2 Polymerase reaction buffer (Clontech) | = 2 μl |
| 100 ng of human genomic DNA | = 1 μl |
| 10 mM dNTP | = 0.4 μl |
| Advantage 2 Polymerase enzyme mix (Clontech) | = 0.2 μl |
| Forward Primer (10 μM) | = 0.4 μl |
| Reverse Primer (10 μM) | = 0.4 μl |
| Water | = 15.6 μl |
| Amplification profile: | |

| | | |
|---|---|---|
| 94° C. - 2 min. | | 1 cycle |
| 94° C. - 30 sec. | | |
| 70° C. - 45 sec. | {close oversize bracket} | 10 cycles |
| 72° C. - 1 min. | | |
| 94° C. - 30 sec. | | |
| 64° C. - 45 sec. | {close oversize bracket} | 35 cycles |
| 72° C. - 1 min. | | |

Sequencing of PCR Products

The PCR products were purified by Solid Phase Reversible Immobilization using the protocol developed by the Whitehead Genome Center. A detailed protocol can be found at http://www.genome.wi.mit.edu/sequencing/protocols/pure/SPRI_pcr.html.

Briefly, five ill of carboxyl coated magnetic beads (10 mg/ml) and 60 μof HYB BUFFER (2.5M NaCl/20% PEG 8000) were added to each PCR reaction mixture (20 μl). The reaction mixture was mixed well and incubated at room temperature (RT) for 10 min. The microtitre plate was placed on a magnet for 2 min and the beads washed twice with 150 μl of 70% EtOH. The beads were air dried for 2 min and the DNA was eluted in 25 μl of distilled water and incubated at RT for 5 min. The beads were magnetically separated and the supernatant removed for testing and sequencing.

The purified PCR products were sequenced in both directions using the primer sets described previously or those listed, in the 5′ to 3′ direction, below.

Accession Number: AC004525

| | | |
|---|---|---|
| Fragment 1 | | |
| Forward Primer | | |
| 32865-32882 | GCGCTGGCCCTCAACTTT | (SEQ ID NO:336) |
| Reverse Primer | | |
| Complement of 33283-33264 | GTCCCTGGAGATGGGACCTC | (SEQ ID NO:337) |
| Fragment 2 | | |
| Forward Primer | | |
| 35598-35617 | GCCCCCAGATCTGTCCTCAC | (SEQ ID NO:33 8) |
| Reverse Primer | | |
| Complement of 36013-35994 | GGAAAATACAGGCGGCTTCC | (SEQ ID NO:339) |
| Fragment 3 | | |
| Forward Primer | | |
| 37182-37203 | GGCTCTGAATCTGTGTGGTGCT | (SEQ ID NO:340) |
| Reverse Primer | | |
| Complement of 37639-37620 | AGCCAGGTGAGAAGCCAGGT | (SEQ ID NO:341) |
| Fragment 4 | | |
| Forward Primer | | |
| 43266-43285 | GGCCTGAACAGGACGAACAA | (SEQ ID NO:342) |
| Reverse Primer | | |
| Complement of 43687-43668 | GGCAGGATTGCCATTAGAGG | (SEQ ID NO:343) |
| Fragment 5 | | |
| Forward Primer | | |
| 49639-49660 | TGAGTCAGTGGTTTGACCTCCA | (SEQ ID NO:344) |
| Reverse Primer | | |
| Complement of 49999-49980 | GCCTCTGTCTCCCCTGCAAC | (SEQ ID NO:345) |
| Fragment 6 | | |
| Forward Primer | | |
| 51423-51442 | CCATTTTGCCATCGACCAC | (SEQ ID NO:346) |
| Reverse Primer | | |
| Complement of 51818-51799 | CTGCCGTCCCTTTGAAGGCTA | (SEQ ID NO:347) |
| Fragment 7 | | |
| Forward Primer | | |
| 52932-52953 | CCCTACCCTCAGGGATTTCTCA | (SEQ ID NO:348) |
| Reverse Primer | | |
| Complement of 53474-53455 | CCCATTCTCCTCTCCGAGCA | (SEQ ID NO:349) |
| Fragment 8 | | |
| Forward Primer | | |
| 53280-53299 | ATCAGCGTGGTGCGATGTGT | (SEQ ID NO:350) |
| Reverse Primer | | |
| Complement of 53756-53737 | ACCCAGCTCTCTGGGACACG | (SEQ ID NO:351) |
| Fragment 9 | | |
| Forward Primer | | |
| 53548-53568 | GGGATGAGTTCCCAAGTGCAG | (SEQ ID NO:352) |
| Reverse Primer | | |
| Complement of 54048-54029 | TGGCAAGCAGGCTTGAGAAG | (SEQ ID NO:353) |
| Fragment 10 | | |
| Forward Primer | | |
| 53841-53860 | CCCCAACCTGAGCCAGAAAC | (SEQ ID NO:354) |
| Reverse Primer | | |
| Complement of 54330-54311 | TGTCCACAAGGGGGTCTGTG | (SEQ ID NO:355) |
| Fragment 11 | | |
| Forward Primer | | |
| 54083-54102 | GGCTAGCAGTGGGGAAGAGG | (SEQ ID NO:356) |
| Reverse Primer | | |
| Complement of 54617-54600 | ATTGCCAGGGGCAGGATG | (SEQ ID NO:357) |
| Fragment 12 | | |
| Forward Primer | | |
| 54422-54441 | CCCTGTCATGGCCAGTCCTT | (SEQ ID NO:358) |
| Reverse Primer | | |
| Complement of 54929-54910 | GCGACCCAGTGCCCTCTACT | (SEQ ID NO:359) |
| Fragment 13 | | |
| Forward Primer | | |
| 54694-54715 | TGCATGTCCTCTTGTTGCTGAG | (SEQ ID NO:360) |
| Reverse Primer | | |
| Complement of 55213-55194 | CAATGACCACCCTCCCTGAA | (SEQ ID NO:361) |
| Fragment 14 | | |
| Forward Primer | | |
| 55273-55291 | CGGCTGTCAAGGGGTGTTC | (SEQ ID NO:362) |
| Reverse Primer | | |
| Complement of 55769-55750 | CCAAACCCAGACGGCAAGTT | (SEQ ID NO:363) |

Analysis of Sequences for Polymorphic Sites

Sequences were analyzed for the presence of polymorphisms using the Polyphred program (Nickerson et al., Nucleic Acids Res. 14:2745-2751, 1997). The presence of a polymorphism was confirmed on both strands. The polymorphisms and their locations in the IL4Rα gene are listed in Table 3 below.

### Example 1B

This example illustrates examination of the IL4Rα gene for polymorphic sites in the following target regions: 1000 base pairs upstream of the ATG start codon; each of the exons, including approximately 100 base pairs on either side of the exon; and 500-1000 base paris downstream of the termination codon.

Amplification of the Target Regions PCR primers, which were designed based on the nearly complete IL4R genomic sequence reported in the GenBank database (Accession No: AC004525), are set forth below:

| | | |
|---|---|---|
| Promoter | | |
| Forward Primer | | |
| 30094-30117 | AAACCCCTGGACTCCAAGTGATCC | (SEQ ID NO:364) |
| Reverse Primer | | |
| Complement of 30842-30819 | AAGCGATTCTTCTGCCTCAGCCTC | (SEQ ID NO:365) |
| PCR product 749 nt | | |
| Exon 1 | | |
| Forward Primer | | |
| 30517-30540 | GGACAGTTGTTGTGTAGCTCACCC | (SEQ ID NO:366) |
| Reverse Primer | | |
| Complement of 31409-31431 | CTATGTTGCCCAAGCTGACCTC | (SEQ ID NO:367) |
| PCR product 893 nt | | |
| Exon 2 | | |
| Forward Primer | | |
| 32871-32890 | GCCCTCAACTTTGCCTGCAC | (SEQ ID NO :368) |
| Reverse Primer | | |
| Complement of 33362-33340 | AGTCCAGTATTCCAGCCGTATCC | (SEQ ID NO:369) |
| PCR product 492 nt | | |
| Exon 3 | | |
| Forward Primer | | |
| 35623-35644 | TGATCGGGAAGCTGGAAGAGTC | (SEQ ID NO:370) |
| Reverse Primer | | |
| Complement of 36070-36049 | CGTTTCAAAGGTGGGAGGACTG | (SEQ ID NO:371) |
| PCR product 448 nt | | |
| Exon 4 | | |
| Forward Primer | | |
| 37230-37250 | CGACCAAAAATCTGGGTGGTG | (SEQ ID NO:372) |
| Reverse Primer | | |
| Complement of 37668-37648 | CAGGAAGCAAAGGGACTTGCC | (SEQ ID NO:373) |
| PCR product 439 nt | | |
| Exon 5 | | |
| Forward Primer | | |
| 43307-43328 | TCTTAAACATGGTGGGGTCAGC | (SEQ ID NO:374) |
| Reverse Primer | | |
| Complement of 43765-43745 | CATGGAAATTTGTGGGCTTTGTG | (SEQ ID NO:375) |
| PCR product 459 nt | | |
| Exon 6 | | |
| Forward Primer | | |
| 46507-46528 | ATGTGCAAGAGGGAGAGTGGTG | (SEQ ID NO:376) |
| Reverse Primer | | |
| Complement of 46870-46849 | TGACTGAGAGGACTGCAAAGGG | (SEQ ID NO:377) |
| PCR product 364 nt | | |
| Exon 7 | | |
| Forward Primer | | |
| 49672-49695 | GCCTGATCTCTGATGCCAAATAAG | (SEQ ID NO:378) |
| Reverse Primer | | |
| Complement of 49964-49983 | TTTGCCATFCCAGAAGCCAG | (SEQ ID NO:379) |
| PCR product 293 nt | | |
| Exon 8 | | |
| Forward Primer | | |
| 51524-51546 | GATCTGTGTGATGTCGAGGCTTG | (SEQ ID NO:380) |
| Reverse Primer | | |
| Complement of 51845-51825 | GTCCACGTTTCCAGAACACCC | (SEQ ID NO:381) |
| PCR product 322 nt | | |
| Exon 9 Fragment 1 | | |
| Forward Primer | | |
| 52912-52933 | CGAAATCCCAAAGACACAGACC | (SEQ ID 140:382) |
| Reverse Primer | | |
| Complement of 53722-53701 | GAGTTGCTGAAGCTGCGGTAAG | (SEQ ID NO:3 83) |
| PCR product 811 nt | | |
| Exon 9 Fragment 2 | | |
| Forward Primer | | |
| 53352-53374 | GAAAAAGGGAGCTTTCTGTGCATC | (SEQ ID NO:384) |
| Reverse Primer | | |
| Complement of 54172-54153 | AACAAGGGGACAGGGACTGG | (SEQ ID 140:385) |
| PCR product 821 nt | | |
| Exon 9 Fragment 3 | | |
| Forward Primer | | |
| 53854-53874 | CAGAAACCTGGGAGCAGATCC | (SEQ ID NO:386) |
| Reverse Primer | | |
| Complement of 54711-54688 | GCAACAAGAGGACATGCACCTAAG | (SEQ ID 140:387) |
| PCR product 858 nt | | |
| Exon 9 Fragment 4 | | |
| Forward Primer | | |
| 54266-54288 | AAAGGTAGAGGACATGCCAAAGC | (SEQ ID NO:388) |
| Reverse Primer | | |
| Complement of 55007-54987 | GGAGCAGCCAACAACTCGTTC (SEQ ID 140:389) | |
| PCR product 742 nt | | |

These primer pairs were used in PCR reactions containing genomic DNA isolated from immortalized cell lines for a reference population of 70 human individuals. The PCR reactions were carried out under the following conditions:

| | |
|---|---|
| Reaction volume | = 20 μl |
| 10 × Advantage 2 Polymerase reaction buffer (Clontech | = 2 μl |
| 100 ng of human genomic DNA | = 1 μl |
| 10 mM dNTP | = 0.4 μl |
| Advantage 2 Polymerase enzyme mix (Clontech) | = 0.2 μl |
| Forward Primer (10 μM) | = 0.4 μl |
| Reverse Primer (10 μM) | = 0.4 μl |
| Water | = 15.6 μl |
| Amplification profile: | |

| | | |
|---|---|---|
| 94° C. - 2 min. | | 1 cycle |
| 94° C. - 30 sec. | | |
| 70° C. - 45 sec. | {close oversize bracket} | 10 cycles |
| 72° C. - 1 min. | | |
| 94° C. - 30 sec. | | |
| 64° C. - 45 sec. | {close oversize bracket} | 35 cycles |
| 72° C. - 1 min. | | |

Sequencing of PCR Products

The PCR products were purified by Solid Phase Reversible Immobilization using the protocol developed by the Whitehead Genome Center. A detailed protocol can be found at http://www.genome.wi.mit.edu/sequencing/protocols/pure/SPRI_pcr.html.

Briefly, five μl of carboxyl coated magnetic beads (10 mg/ml) and 60 μl of HYB BUFFER (2.5M NaCl/20% PEG 8000) were added to each PCR reaction mixture (20 μl ). The reaction mixture was mixed well and incubated at room temperature (RT) for 10 min. The microtitre plate was placed on a magnet for 2 min and the beads washed twice with 150 μl of 70% EtOH. The beads were air dried for 2 min and the DNA was eluted in 25 μl of distilled water and incubated at RT for 5 min. The beads were magnetically separated and the supernatant removed for testing and sequencing.

The purified PCR products were sequenced in both directions using the primer sets described previously or those listed, in the 5′ to 3′ direction, below.

| | | |
|---|---|---|
| Promoter Fragment 1 | | |
| Forward Primer | | |
| 30312-30334 | GCTCATTTAATCCCCACAACACC | (SEQ ID NO:390) |
| Reverse Primer | | |
| Complement of 30791-30769 | CCACCACACCTGGCTAATTTTTG | (SEQ ID NO:391) |
| Promoter Fragment 2 | | |
| Forward Primer | | |
| 30529-30551 | TGTAGCTCACCCTCTGGACTTTG | (SEQ ID NO:392) |
| Reverse Primer | | |
| Complement of 30990-30971 | AATATGCAACCCTCCCCTGC | (SEQ ID NO:393) |
| Exon 1 | | |
| Forward Primer | | |
| 30824-30846 | TGAGGCAGAAGAATCGCTTGAAC | (SEQ ID NO:394) |
| Reverse Primer | | |
| Complement of 31261-31240 | ACTTGTCATTGGCTGTCCCCTC | (SEQ ID NO:395) |
| Exon 2 | | |
| Forward Primer | | |
| 32880-32900 | TTTGCCTGCACTGTGCTTTTG | (SEQ lED NO:396) |
| Reverse Primer | | |
| Complement of 33234-33212 | CCATACTCAGCATCCTGCACTCC | (SEQ ID NO:397) |
| Exon 5 | | |
| Forward Primer | | |
| 43330-43349 | AACGACAGCAACCAGGGTGG | (SEQ ID NO:398) |
| Reverse Primer | | |
| Complement of 43704-43682 | CAGCAGGTGTATCTAATGGCAGG | (SEQ ID NO:399) |
| Exon 6 | | |
| Forward Primer | | |
| 46520-46541 | AGAGTGGTGGGGAGATGAGGTG | (SEQ ID NO:400) |
| Reverse Primer | | |
| Complement of 46857-46837 | TGCAAAGGGGCAGACTAGAGG | (SEQ ID NO:40 1) |
| Exon 7 | | |
| Forward Primer | | |
| 49708-49729 | CGACCACTTTTATGGGAGGAGC | (SEQ ID NO:402) |
| Reverse Primer | | |
| Complement of 49927-49905 | CCAGGTGTTCTGAACCACACTTC | (SEQ ID NO:403) |
| Exon 8 | | |
| Forward Primer | | |
| 51528-51550 | TGTGTGATGTCGAGGCTTGTACC | (SEQ ID NO:404) |
| Reverse Primer | | |
| Complement of 51779-51758 | GAATGCAGGGAAGAGAAGGCAG | (SEQ ID NO:405) |
| Exon 9 Fragment 1 | | |
| Forward Primer | | |
| 53017-53038 | GCCATCAGGACATGGTGATTTC | (SEQ ID NO:406) |
| Reverse Primer | | |
| Complement of 53539-53518 | TGAGCACTCGTACTTCCCGAAG | (SEQ ID NO:407) |
| Exon 9 Fragment 2 | | |
| Forward Primer | | |
| 53378-53399 | TGAGAGCAGCAGGGATGACTTC | (SEQ ID NO:408) |
| Reverse Primer | | |
| Complement of 53948-53926 | AAACTCCTGATAGCCACTGGTGG | (SEQ ID NO:409) |
| Exon 9 Fragment 3 | | |
| Forward Primer | | |
| 53869-53868 | AGATCCTCCGCCGAAATGTC | (SEQ ID NO:410) |
| Reverse Primer | | |
| Complement of 54583-54560 | TTACTCTTCTCTGAGATGCCCGAG | (SEQ ID NO:411) |
| Exon 9 Fragment 4 | | |
| Forward Primer | | |
| 54334-54355 | TGGGCAGTGGCATTGTCTACTC | (SEQ ID NO:412) |
| Reverse Primer | | |
| Complement of 54769-54750 | TTCCAGGAGGTGGCATTTCC | (SEQ ID NO:413) |

Sequencing reactions were performed using the Big-Dye terminator kit from PE Biosystems (Foster City, Calif.) according to the manufacturer's instructions. The sequencing products were analyzed on an ABI 477 automated sequencer (PE Biosystems, Foster City, Cailf.).

Analysis of Sequences for Polymorphic Sites Sequences were analyzed for the presence of polymorphisms using the Polyphred program (Nickerson et al., 14 Nucleic Acids Res. 2745-2751, 1997). The presence of a polymorphism was confirmed on both the strands. The polymorphisms and their locations in the IL4Rα gene are listed in Table 3 below.

TABLE 3

Polymorphic Sites Identified in the IL4Rα Gene

| | | | | | |
|---|---|---|---|---|---|
| Polymorphic | Nucleotide Position in | Nucleotide Position in | Reference | Variant | |
| Site Number | GenBank Accession | | Allele | Allele | Example |

| | | | | | |
|---|---|---|---|---|---|
| PS1 | 97137(Acc#AC004525) | 32884 | A | G | 1B |
| PS2 | 97118(Acc#AC004525) | 32903 | C | T | 1A |
| PS3 | 97060(Acc#AC004525) | 32961 | G | T | 1A |
| PS4 | 96886(Acc#AC004525) | 33135 | G | C | 1A |
| PS5^{R} | 94272(Acc#AC004525) | 35749 | A | G | 1A, 1B |
| PS6 | 94258(Acc#AC004525) | 35763 | C | T | 1A, 1B |
| PS7 | 94251 (Acc#AC004525) | 35770 | G | A | 1A |
| P58 | 94204(Acc#AC004525) | 35817 | T | C | 1A |
| PS9 | 94116(Acc#AC004525) | 35905 | C | T | 1B |
| PS10 | 94077(Mc#AC004525) | 35944 | C | T | 1A |
| PS11 | 94063(Acc#AC004525) | 35958 | G | A | 1B |
| PS12 | 92691(Acc#AC004525) | 37330 | C | A | 1A |
| PS13 | 92548(Acc#AC004525) | 37473 | C | T | 1A |
| P514 | 92435(Acc#AC004525) | 37586 | C | T | 1A |
| PS15 | 92430(Acc#AC004525) | 37591 | C | A | 1A |
| PS16 | 92417(Acc#AC004525) | 37604 | A | T | 1A |
| PS17 | 92377(Acc#AC004525) | 37644 | C | A | 1A |
| PS18 | 92343(Acc#AC004525) | 37678 | C | T | 1A |
| PS19 | 86575(Acc#AC004525) | 43446 | G | A | 1A |
| PS20 | 86318(Acc#AC004525) | 43703 | T | C | 1A |
| PS21 | 77013(Acc#AC004525) | 53008 | A | C | 1A |
| PS22 | 76922(Acc#AC004525) | 53099 | C | T | 1B |
| PS23 | 76868(Acc#AC004525) 53153 | T | C | 1A | |
| PS24^{R} | 76608(Acc#AC004525) | 53413 | A | C | 1A |
| PS25 | 76565(Mc#AC004525) 53456 | C | T | 1A, 1B | |
| PS26^{R} | 76516(Mc#AC004525) | 53505 | T | C | 1A, 1B |
| PS27 | 76514(Acc#AC004525) | 53507 | C | T | 1A |
| P528 | 76508(Acc#AC004525) | 53513 | T | C | 1A |
| PS29^{R} | 76300(Mc#AC004525) 53721 | T | C | 1A, 1B | |
| PS30 | 76106(Acc#AC004525) 53915 | C | T | 1A | |
| PS31^{R} | 76080(Acc#AC004525) | 53941 | A | C | 1A, 1B |
| PS32 | 76072(Acc#AC004525) | 53949 | G | A | 1A |
| PS33 | 75784(Acc#AC004525) | 54237 | C | T | 1A |
| PS34 | 75553(Acc#AC004525) | 54468 | T | C | 1A, 1B |
| PS35 | 75410(Acc#AC004525) | 54611 | T | C | 1A |
| PS36 | 75323(Acc#AC004525) | 54698 | T | C | 1A, 1B |
| PS37 | 75321(Acc#AC004525) | 54700 | T | C | 1A |
| PS38 | 75280(Acc#AC004525) | 54741 | C | T | 1A |
| PS39 | 75241(Acc#AC004525) | 54780 | C | G | 1A |
| PS40 | 74938(Acc#AC004525) | 55083 | A | G | 1A |
| PS41 | 74879(Acc#AC004525) | 55142 | G | A | 1A |
| PS42^{R} | 74693(Acc#AC004525) | 55328 | G | A | 1A |
| PS43^{R} | 74591(Acc#AC004525) | 55430 | C | T | 1A |
| PS44 | 74482(Acc#AC004525) | 55539 | C | T | 1A |
| PS45 | 14263(Acc#AC004525) | 55758 | G | A | 1A |

^{R}Previously reported in the literature

### Example 2

This example illustrates analysis of the IL4Rα polymorphisms identified in the Index Repositories for human genotypes and haplotypes for all polymorphic sites except PS1, PS9, PS11, PS21, PS22, and PS23.

A sampling of different genotypes containing these polymorphisms that were observed in these reference populations are shown in Table 4 below, with the haplotype pair indicating the combination of haplotypes determined for the individual using the haplotype derivation protocol described below. In Table 4, homozygous positions are indicated by one nucleotide and heterozygous positions are indicated by two nucleotides. Missing nucleotides in any given genotype in Table 4 can typically be inferred based on linkage disequilibrium and/or Mendelian inheritance.

TABLE 4
Genotypes and HAP Pairs Observed for the IL4Rα Gene

| | PS | PS | PS | PS | PS | PS | PS | PS | PS | PS | PS | PS | PS | PS | PS | PS | PS | PS | PS | PS | PS | PS | PS | PS | PS | PS | PS | PS | PS | PS | |
|---|---|---|---|---|---|---|---|---|---|---|---|---|---|---|---|---|---|---|---|---|---|---|---|---|---|---|---|---|---|---|---|
| | 2 | 3 | 4 | 5 | 6 | 7 | 8 | 10 | 12 | 13 | 14 | 15 | 16 | 17 | 18 | 19 | 20 | 24 | 25 | 26 | 27 | 28 | 29 | 30 | 31 | 32 | 33 | 34 | 35 | 36 | |
| 1 | C | G | G | A | C | G | T | C | G | C | C | G | A | C | G | T | A | G | T | C | T | T | C | A | G | C | T | T | T | | |
| 2 | C | G | G | A | C | G | T | C | G | C | C | G | A | C | C | G | T | A | G | T | C | T | T | C | A | G | C | T | T | T | |
| 3 | C | G | G | G/A | C | G | T | C/T | G | C | C | G | A | C | C/T | G | C/T | A | G | T | C | T | T | C | A/G | G | C | T | T | T/C | |
| 4 | C/T | G | G | G/A | C | G | T | C | G/A | C/T | C | G | A | C | C | G | C/T | A | G | T | C | T | T | C | A | G | C | T | T | T | |
| 5 | C | G | G | G/A | C | G | T | C/T | G | C | C | G | A | C | C/T | G | C/T | A | G | T | C | T | T | C | A | G | C | T | T | T | |
| 6 | C | G | G | A | C | G | T | C | G | C | C | G | A | C | C/T | G | C/T | A | G | T | C | T | T | C | A/G | G | C | T | T | T/C | |
| 7 | C | G | G | A | C | G | T | C | G | C | C | G | A | C | C/T | G | C/T | A | G | T | C | T | T | C | A | G | C | T | T | T | |
| 8 | C/T | G | G | G/A | C | G | T | C/T | G | C | C | G | A | C | C/T | G | C/T | A | G | T | C | T | T | C | A | G | C | T | T | T | |
| 9 | C | G | G | A | C | G | T | C | G | C | C | G | A | C | C | G | T | A | G | T | C | T | T | C | A | G | C | T | T | T | |
| 10 | C | G | G | G/A | C | G | T | C | G | C/T | C | G | A | C | C | G | C/T | A | G | T | C | T | T | C | A | G | C | T | T | T | |
| 11 | C | G | G | G/A | C | G | T | C/T | G | C | C | G | A | C | C/T | G | C/T | A | G | T | C | T | T | C | A | G | C | T | T | T | |
| 12 | C/T | G | G | G | C | G | T | T | G | C | C | G | A | C | T | G | C | A/C | G/T | T/C | C | T/C | T/C | C | G | G | C | T | T | T/C | |
| 13 | C | G | G | G | C | G | T | T | G | C/T | C | G | A | C | C | G | C/T | A/C | G/T | T | C | T/C | T | C | A/G | G | C | T/G | T | T | |
| 14 | — | — | — | G/A | C | G | T | C/T | G | C | C | G | A | C | C/T | G | C/T | A/C | G/T | T/C | C | T/C | T/C | C | A/G | G | C | T | T | T | |
| 15 | C | G | G | G | C | G | T | T | G | C | C | G | A | C | T | G | C | C | T | C | C | C | C | C | G | G | C | T | T | T | |
| 16 | C | G | G | G/A | C | G | T | C | G | C | C | G | A | C | C | G | C | C | T | T/C | C | C | C | C | G | G | C | T | T | T | |
| 17 | C | G | G | G | C | G | T | C | G | C | C | G | A | C | C | G | C | A | G | T | C | T | T | C | G | G | C | T | T | T | |
| 18 | C | G | G/C | G | C | G | T | T | G | C | C | G | A | C | C/T | G | C | C | T | T | C | C | T/C | C | G | GT | C | T/G | T/C | T/C | T/C |
| 19 | C | G | G | G/A | C | G | T | C/T | G | C | C | G | A | C | C/T | G | C | A/C | G/T | T | C | T/C | T/C | C | G | G | C | T | T/C | | |
| 20 | C/T | G | G | G/A | C | G | T | C | G/A | C/T | C | G/A | A | C | C | G | C/T | A | G | T | C | T | T | C | A | G | C | T | T | T | T |
| 21 | C | G | G | A | C | G | T | C | G | C | C | G/A | A | C/A | C | G | C/T | A/C | G/T | T/C | C | T/C | T/C | C | A/G | G | C | T | T | T | |
| 22 | C | G | G | G | C | G | T | C | G | C/T | C | G | A | C | C | G | C | C | T | T | C | C | T/C | C | G | T | C | T/G | T | T | |
| 23 | C | G | G | G | C | G | T | T | G | C | C | G | A | C | T | G | C | A | G | T | C | T | T | C | A/G | G | C | T | T | T/C | T |
| 24 | C | G | G | G/A | C | G | T | C | G | C | C | G | A | C | C | G | C | A/C | G/T | T | C | T/C | C | C | G | G | C | T/G | T | T | |
| 25 | C | G | G | A | C | G | T | C | G | C | C | G | A | C | C/T | G | C | C | T | T | C | C | T/C | C | G | G/A | C | T/G | T | T | T/C |
| 26 | C | G | G | A | C | G | T | C | G | C | C | G | A | C | C/T | G | C | C | T | T/C | C | C | T/C | C | G | G/A | C | T/G | T | T | |
| 27 | C/T | G | G | G | C | G | T | C/T | G | C | C | G | A | C | C/T | G | C | A/C | G/T | T | C | T/C | T | C | G | G/A | C | G | T | T/C | |
| 28 | T | G | G | G | C | G | T | C | G | C | C | G | A | C | C | G | C | A | G | T | C | T | T | C | G | G | — | G | T | C | |
| 29 | C/T | G | G | G | C | G | T | C/T | G | C | C | G | A | C | C/T | G | C | A | G | T | C | T | T | C | A/G | G | C | T/G | T | T/C | |
| 30 | C | G | G | A | C | G/A | T | C | G | C | C | G | A | C/A | C | G | C/T | A/C | G/T | T/C | C | T/C | T/C | C | A/G | G | C | T | T | T | |
| 31 | C | G | G | G/A | C | G | T | C/T | G | C | C | G | A | C | C/T | G | C/T | A | G | T | C | T | T | C | A | G | C | T | T | T | |
| 32 | C/T | G | G | G | C | G | T | T | G | C | C | G | A | C | T | G | C | A/C | G/T | T/C | C | T/C | T/C | C | A/G | G | C | T | T | T | |
| 33 | C | G | G | G | C | G | T | T | G | C | C | G | A | C | T | G | C | A/C | G/T | T | C | T/C | T | C | A/G | G/A | C | T/G | T | T | |
| 34 | C | G | G | G/A | C | G | T | C/T | G | C | C | G | A | C | C/T | G | C/T | A | G | T | C | T | T | C | A/G | G | C | T | T | T | T |
| 35 | C | G | G | G/A | C | G | T | C/T | G | C | C | G | A | — | — | G | C/T | A | G | T | C | T | T | C/T | A | G | C | T | T | T | |
| 36 | C | G | G | G/A | C | G | T | C/T | G | C | C | G | A | — | — | G | C | A | G | T | C | T | T | C | A | G | C | T | T | T | T |
| 37 | C | G | G | G | C | G | T | T | G | C | C | G | A | C | T | G | C | A | G | T | C | T | T | C | A | G | C | T | T | T | |
| 38 | C | G | G | — | — | — | T | T | G | C | C | G | A | C | C/T | G | C/T | A | G | T | C | T | T | C | A | G | C | T | T | T | |
| 41 | C | G | G | G | C | G | T | T | G | C | C/T | G | A | C | C | G | C/T | A/C | G/T | T | C | T/C | T | C | A/G | G | C | T/G | T | T/C | |
| 42 | C/T | G | G | G/A | C/T | G | T/C | C | G | C | C | G | A | C | C | G | C | C | T | T | C/T | C | T/C | C | G | G | C | T/G | T | T/C | |

| | | | | | | | | | |
|---|---|---|---|---|---|---|---|---|---|
| PS | PS | PS | PS | PS | PS | PS | PS | PS | HAP |
| 37 | 38 | 39 | 40 | 41 | 42 | 43 | 44 | 45 | PAIRS |

| | | | | | | | | | | | |
|---|---|---|---|---|---|---|---|---|---|---|---|
| T | C | C | A | G | G | C | C | G | 2 | 2 | |
| T | C | C | G/A | G | A/G | C | C | A/G | 2 | 3 | |
| T | C | C | A | G | G | C | C | G | 2 | 4 | |
| T | C | C | A | G | G | C | C | G | 2 | 15 | |
| T | C | C | A | G | G | C | C | G | 2 | 16 | |
| T | C | C | A | G | G | C | C | G | 2 | 36 | |
| T | C | C | A | G | G | C | C | G | 2 | 39 | |
| T | C | C | G/A | G | A/G | C | C | A/G | 2 | 48 | |
| T | C | C | G/A | G | A/G | C | C | A/G | 3 | 2 | |
| T | C | C | G | G | A | C | C | A | 3 | 24 | |
| T | C | C | G | G | A | C | C | A | 3 | 44 | |
| T | C | C | G/A | G | A/G | C | C | G | 4 | 5 | |
| T | C | C | G | G | A | C | C | A | 4 | 46 | |
| T | C | C | A | G | G | C | C | G | 7 | 2 | |
| T | C | C | A | G | G | C | C | G | 7 | 7 | |
| T | C/T | C | G | G | A | C | C | A | 9 | 10 | |
| T | C | C | A | G | G | C | C | G | 12 | 12 | |
| C | C | G | G | A | T | C | A | 13 | 14 | | |
| T | T/C | C | C | G/A | G | A/G | C/T | C | A/G | 13 | 38 |
| C | C | A | G | G | C | C | G | 15 | 51 | | |
| T | C | C | A | G | G | C | C | G | 17 | 22 | |
| T | C/T | C | G | G | A | C | C | — | 21 | 10 | |
| C | C | G/A | G | G | C | C | G | 23 | 4 | | |
| T/C | C | C/G | G | G | A | C/T | C | A | 28 | 31 | |
| C | C | G | G | A | T | C | A | 29 | 37 | | |
| T | C | C | G | G | A/G | C/T | C | A/G | 32 | 33 | |
| T/C | C | C | G | G | A | C/T | C | A | 41 | 8 | |
| C | C | C | G | G | A | C | C | A | 41 | 41 | |
| T/C | C | C | G | G | A | C | C | A | 41 | 44 | |
| T | C | C | A | G | G | C | C | G | 43 | 22 | |
| T | C | C | G | G | A | C | C | A | 44 | 3 | |
| T | C | C | | G | A | C | C | A/G | 44 | 5 | |
| T | C | C | G | G | A | C/T | C | A | 44 | 8 | |
| C | C | G | G | A | C | C | A | 44 | 30 | | |
| T | C | C | G/A | G | A/G | C | C | A/G | 44 | 34 | |
| C | C | G | G | A | C | C | A | 44 | 40 | | |
| T | C | C | G | G | A | C | C | A | 44 | 44 | |
| T | C | C | G | G | A | C | C | A | 44 | 46 | |
| T | C | C | G | G | A | C/T | C | A | 46 | 50 | |
| T | C | C | G | G | A | T | C | A | 53 | 20 | |

The haplotype pairs shown in Table 4 were estimated from the unphased genotypes using an extension of Clark's algorithm (Clark, A. G. (1990) Mol Bio Evol 7, 111-122), as described in U.S. Provisional Patent Application filed Apr. 19, 2000 and entitled “A Method and System for Determining Haplotypes from a Collection of Polymorphisms”. In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals.

By following this protocol, it was determined that the Index Repositories examined herein and, by extension, the general population contains the 53 human IL4Rα haplotypes shown in Table 5 below.

TABLE 5
A. Haplotypes Observed for the IL4Rα Gene

| HAP | PS | PS | PS | PS | PS | PS | PS | PS | PS | PS | PS | PS | PS | PS | PS | PS | PS | PS | PS | PS | PS | PS | PS | PS | PS | PS | PS | PS | PS | PS | PS |
|---|---|---|---|---|---|---|---|---|---|---|---|---|---|---|---|---|---|---|---|---|---|---|---|---|---|---|---|---|---|---|---|
| ID | 2 | 3 | 4 | 5 | 6 | 7 | 8 | 10 | 12 | 13 | 14 | 15 | 16 | 17 | 18 | 19 | 20 | 24 | 25 | 26 | 27 | 28 | 29 | 30 | 31 | 32 | 33 | 34 | 35 | 36 | |
| 1 | C | G | G | A | C | G | T | C | G | C | C | G | A | C | T | G | C | A | G | T | C | T | T | C | A | G | C | T | T | T | |
| 2 | C | G | G | A | C | G | T | C | G | C | C | G | A | C | C | G | T | A | G | T | C | T | T | C | A | G | C | T | T | T | |
| 3 | C | G | G | A | C | G | T | C | G | C | C | G | A | C | C | G | T | A | G | T | C | T | T | C | A | G | C | T | T | T | |
| 4 | C | G | G | G | C | G | T | T | G | C | C | G | A | C | T | G | C | A | G | T | C | T | T | C | G | G | C | T | T | C | |
| 5 | T | G | G | G | C | G | T | T | G | C | C | G | A | C | T | G | C | C | T | C | C | C | C | C | G | G | C | T | T | T | |
| 6 | C | G | G | G | C | G | T | T | G | C | C | G | A | C | T | A | C | A | G | T | C | T | T | C | A | G | C | T | T | T | |
| 7 | C | G | G | G | C | G | T | T | G | C | C | G | A | C | T | G | C | C | T | C | C | C | C | C | G | G | C | T | T | T | |
| 8 | C | G | G | G | C | G | T | T | G | C | C | G | A | C | T | G | C | C | T | T | C | C | T | C | G | A | C | G | T | T | |
| 9 | C | G | G | A | C | G | T | C | G | C | C | G | A | C | C | G | C | C | T | C | C | C | C | C | G | G | C | T | T | T | |
| 10 | C | G | G | G | C | G | T | C | G | C | C | G | A | C | C | G | C | C | T | T | C | C | C | C | G | G | C | T | T | T | |
| 11 | C | G | G | A | C | G | T | C | G | C | C | G | A | C | C | G | C | A | G | T | C | T | T | C | G | G | C | G | T | C | |
| 12 | C | G | G | G | C | G | T | C | G | C | C | G | A | C | C | G | C | A | G | T | C | T | T | C | G | G | C | T | T | T | |
| 13 | C | G | G | G | C | G | T | T | G | C | C | G | A | C | T | G | C | C | T | T | C | C | C | C | G | G | C | T | C | T | |
| 14 | C | G | C | G | C | G | T | T | G | C | C | G | A | C | C | G | C | C | T | T | C | C | T | C | G | G | C | G | T | C | |
| 15 | T | G | G | G | C | G | T | C | A | T | C | G | A | C | C | G | C | A | G | T | C | T | T | C | A | G | C | T | T | T | |
| 16 | C | G | G | G | C | G | T | T | G | C | C | G | A | C | T | G | C | A | G | T | C | T | T | C | A | G | C | T | T | T | |
| 17 | C | G | G | A | C | G | T | C | G | C | C | A | A | C | C | G | C | C | T | C | C | C | C | C | G | G | C | T | T | T | |
| 18 | C | G | G | A | C | G | T | C | G | C | C | G | A | A | C | G | C | A | G | T | C | T | T | C | A | G | C | T | T | T | |
| 19 | C | G | G | G | C | G | T | C | G | T | C | G | A | C | C | G | C | C | T | T | T | C | C | C | G | G | C | G | T | C | |
| 20 | C | G | G | A | C | G | T | C | G | C | C | G | A | C | C | G | C | C | T | T | T | C | C | C | G | G | C | T | T | T | |
| 21 | C | G | G | G | C | G | T | C | G | T | C | G | A | C | C | G | C | C | T | T | C | C | T | C | G | G | C | G | T | T | |
| 22 | C | G | G | A | C | G | T | C | G | C | C | G | A | A | C | G | T | A | G | T | C | T | T | C | A | G | C | T | T | T | |
| 23 | C | G | G | G | C | G | T | T | G | C | C | G | A | C | T | G | C | A | G | T | C | T | T | C | A | G | C | T | T | T | |
| 24 | C | G | G | G | C | G | T | C | G | T | C | G | A | C | C | G | C | A | G | T | C | T | T | C | A | G | C | T | T | T | |
| 25 | T | G | G | G | C | G | T | C | A | T | C | G | A | C | C | G | C | C | T | T | C | C | C | C | G | G | C | T | C | T | |
| 26 | C | G | G | G | C | G | T | T | G | C | C | G | A | C | T | G | C | C | T | T | C | C | T | C | G | G | C | G | T | C | |
| 27 | C | G | G | G | C | G | T | T | G | C | C | G | A | C | T | G | C | A | G | T | C | T | T | C | A | G | C | T | T | T | |
| 28 | C | G | G | A | C | G | T | C | G | C | C | G | A | C | C | G | C | A | G | T | C | T | C | C | G | G | C | G | T | T | |
| 29 | C | G | G | A | C | G | T | C | G | C | C | G | A | C | C | G | C | C | T | T | C | C | C | C | G | G | C | T | T | T | |
| 30 | C | G | G | A | C | G | T | C | G | C | C | G | A | C | C | G | T | A | G | T | C | T | T | C | G | G | C | T | T | T | |
| 31 | C | G | G | G | C | G | T | C | G | C | C | G | A | C | C | G | C | C | T | T | C | C | C | C | G | G | C | T | T | T | |
| 32 | C | G | G | A | C | G | T | C | G | C | C | G | A | C | C | G | C | C | T | T | C | C | T | C | G | A | C | G | T | T | |
| 33 | C | G | G | A | C | G | T | C | G | C | C | G | A | C | T | G | C | C | T | C | C | C | C | C | G | G | C | T | T | T | |
| 34 | C | G | G | A | C | G | T | C | G | C | C | G | A | C | C | G | T | A | G | T | C | T | T | T | A | G | C | T | T | T | |

B. Haplotypes Observed for the IL4Rα Gene

| HAP | PS | PS | PS | PS | PS | PS | PS | PS | PS | PS | PS | PS | PS | PS | PS | PS | PS | PS | PS | PS | PS | PS | PS | PS | PS | PS | PS | PS | PS | PS | PS |
|---|---|---|---|---|---|---|---|---|---|---|---|---|---|---|---|---|---|---|---|---|---|---|---|---|---|---|---|---|---|---|---|
| ID | 2 | 3 | 4 | 5 | 6 | 7 | 8 | 10 | 12 | 13 | 14 | 15 | 16 | 17 | 18 | 19 | 20 | 24 | 25 | 26 | 27 | 28 | 29 | 30 | 31 | 32 | 33 | 34 | 35 | 36 | |
| 35 | C | G | G | A | C | G | T | C | G | C | C | G | A | C | C | G | T | A | G | T | C | T | T | C | A | G | T | T | T | T | |
| 36 | C | G | G | A | C | G | T | C | G | C | C | G | A | C | T | G | C | A | G | T | C | T | T | C | G | G | C | T | T | C | |
| 37 | C | G | G | A | C | G | T | C | G | C | C | G | A | C | T | G | C | C | T | T | C | C | T | C | G | A | C | G | T | T | |
| 38 | C | G | G | A | C | G | T | C | G | C | C | G | A | C | C | G | C | A | G | T | C | T | T | C | G | G | C | T | T | T | |
| 39 | C | G | G | A | C | G | T | C | G | C | C | G | A | C | T | G | C | A | G | T | C | T | T | C | A | G | C | T | T | T | |
| 40 | C | G | G | A | C | G | T | C | G | C | C | G | A | C | C | G | C | A | G | T | C | T | T | C | A | G | C | T | T | T | |
| 41 | T | G | G | G | C | G | T | C | G | C | C | G | A | C | C | G | C | A | G | T | C | T | T | C | G | G | C | G | T | C | |
| 42 | C | G | G | A | C | A | T | C | G | C | C | G | A | C | C | G | T | A | G | T | C | T | T | C | A | G | C | T | T | T | |
| 43 | C | G | G | A | C | A | T | C | G | C | C | G | A | C | C | G | C | C | T | C | C | C | C | C | G | G | C | T | T | T | |
| 44 | C | G | G | G | C | G | T | T | G | C | C | G | A | C | T | G | C | A | G | T | C | T | T | C | A | G | C | T | T | T | |
| 45 | C | G | G | G | C | G | T | T | G | C | C | G | A | C | C | G | T | A | G | T | C | T | T | C | A | G | C | T | T | T | |
| 46 | C | G | G | G | C | G | T | T | G | C | C | G | A | C | C | G | T | A | G | T | C | T | T | C | A | G | C | T | T | T | |
| 47 | C | G | G | G | C | G | T | T | G | C | C | G | A | C | T | G | C | C | T | C | C | C | C | C | G | G | C | T | T | T | |
| 48 | T | G | G | G | C | G | T | T | G | C | C | G | A | C | T | G | C | A | G | T | C | T | T | C | A | G | C | T | T | T | |
| 49 | C | G | G | G | C | G | T | T | G | T | C | G | A | C | C | G | C | C | T | T | C | C | T | C | G | G | C | G | T | T | |
| 50 | C | G | G | G | C | G | T | T | G | C | T | G | A | C | C | G | C | C | T | T | C | C | T | C | G | G | C | G | T | C | |
| 51 | C | G | G | A | C | G | T | C | G | C | C | A | A | C | C | G | T | A | G | T | C | T | T | C | A | G | C | T | T | T | |
| 52 | T | T | G | G | C | G | T | T | G | C | C | G | A | C | T | G | C | C | T | C | C | C | C | C | G | G | C | T | T | T | |
| 53 | T | G | G | G | T | G | C | C | G | C | C | G | A | C | C | G | C | C | T | T | C | C | T | C | G | G | C | G | T | C | |

A. Haplotypes Observed for the IL4Rα Gene

| PS | PS | PS | PS | PS | PS | PS | PS | PS |
|---|---|---|---|---|---|---|---|---|
| 37 | 38 | 39 | 40 | 41 | 42 | 43 | 44 | 45 |
| T | C | C | G | G | A | C | C | A |
| T | C | C | A | G | G | C | C | G |
| T | C | C | G | G | A | C | C | A |
| T | C | C | A | G | G | C | C | G |
| T | C | C | G | G | A | C | C | G |
| T | C | C | G | G | A | C | C | A |
| T | C | C | A | G | G | C | C | G |
| T | C | C | G | G | A | T | C | A |
| T | C | C | G | G | A | C | C | A |
| T | T | C | G | G | A | C | C | A |
| C | C | C | G | G | A | C | C | A |
| T | C | C | A | G | G | C | C | G |
| C | C | C | G | G | A | T | C | A |
| T | C | C | G | G | A | T | C | A |
| T | C | C | A | G | G | C | C | G |
| T | C | C | A | G | G | C | C | G |
| T | C | C | A | G | G | C | C | G |
| T | C | C | G | G | A | C | C | A |
| T | C | C | G | G | A | T | C | A |
| T | C | C | G | G | A | T | C | A |
| T | C | C | G | G | A | C | C | A |
| T | C | C | A | G | G | C | C | G |
| T | C | C | G | G | G | C | C | G |
| T | C | C | G | G | A | C | C | A |
| C | C | C | G | G | A | T | C | A |
| T | C | C | G | G | A | T | C | A |
| T | C | C | G | G | A | T | T | A |
| C | C | G | G | G | A | T | C | A |
| C | C | C | G | G | A | T | C | A |
| T | C | C | G | G | A | C | C | A |
| T | C | C | G | G | A | C | C | A |
| T | C | C | G | G | A | T | C | A |
| T | C | C | G | G | G | C | C | G |
| T | C | C | A | G | G | C | C | C |

B. Haplotypes Observed for the IL4Rα Gene

| PS | PS | PS | PS | PS | PS | PS | PS | PS |
|---|---|---|---|---|---|---|---|---|
| 37 | 38 | 39 | 40 | 41 | 42 | 43 | 44 | 45 |
| T | C | C | G | G | A | C | C | A |
| T | C | C | A | G | G | C | C | G |
| T | C | C | G | G | A | T | C | A |
| T | C | C | A | G | G | C | C | G |
| T | C | C | A | G | G | C | C | G |
| T | C | C | G | G | A | C | C | A |
| C | C | C | G | G | A | C | C | A |
| T | C | C | G | G | A | C | C | A |
| T | C | C | A | G | G | C | C | G |
| T | C | C | G | G | A | C | C | A |
| T | C | C | A | G | G | C | C | G |
| T | C | C | G | G | A | C | C | A |
| T | C | C | G | G | A | C | C | G |
| T | C | C | G | G | A | C | C | A |
| T | C | C | G | G | A | C | C | A |
| T | C | C | G | G | A | T | C | A |
| T | C | C | A | G | G | C | C | G |
| T | C | C | G | G | A | C | C | G |
| T | C | C | G | G | A | T | C | A |

In view of the above, it will be seen that the several advantages of the invention are achieved and other advantageous results attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinency of the cited references.

What is claimed is:

1. An isolated polynucleotide comprising a nucleotide sequence selected from the group consisting of:
(a) a first nucleotide sequence which is a polymorphic variant of a reference sequence for Interleukin 4 Receptor Alpha(IL4Rα) gene or a fragment thereof, wherein the reference sequence comprises SEQ ID NO:1, and the polymorphic variant comprises an IL4Rα isogene defined by a haplotype selected from the group consisting of haplotypes 1-53 in Table 5; and
(b) a second nucleotide sequence which is complementary to the first nucleotide sequence.

3. The isolated polynucleotide of claim 1 which is a DNA molecule and comprises both the first and second nucleotide sequences and further comprises expression regulatory elements operably linked to the first nucleotide sequence.

4. A recombinant organism transformed or transfected with the isolated polynucleotide of claim 1, wherein the organism expresses an IL4Rα protein encoded by the first nucleotide sequence.

5. The recombinant organism of claim 4 which is a nonhuman transgenic animal.

6. The isolated polynucleotide of claim 1, wherein the first nucleotide sequence is a polymorphic variant of a fragment of the IL4Rα isogene, the fragment comprising one or more polymorphisms selected from the group consisting of: guanine at PS1, thymine at PS2, thymine at PS3, cytosine at PS4, thymine at PS6, adenine at PS7, cytosine at PS8, thymine at PS9, thymine at PS10, adenine at PS11, adenine at PS12, thymine at PS13, thymine at PS14, adenine at PS15, thymine at PS16, adenine at PS17, thymine at PS18, adenine at PS19, cytosine at PS20, cytosine at PS21, thymine at PS22, cytosine at PS23, thymine at PS25, thymine at PS27, cytosine at PS28, thymine at PS30, adenine at PS32, thymine at PS33, guanine at PS34, cytosine at PS35, cytosine at PS36, cytosine at PS37, thymine at PS38, guanine at PS39, guanine at PS40, adenine at PS41, thymine at PS44, and adenine at PS45.

7. An isolated polynucleotide comprising a nucleotide sequence which is a polymorphic variant of a reference sequence for the IL4Rα cDNA or a fragment thereof, wherein the reference sequence comprises SEQ ID NO:2 and the polymorphic variant comprises adenine or guanine at a position corresponding to nucleotide 223, cytosine or thymine at a position corresponding to nucleotide 237, guanine or adenine at a position corresponding to nucleotide 244, thymine or cytosine at a position corresponding to nucleotide 291, cytosine or thymine at a position corresponding to nucleotide 501, guanine or adenine at a position corresponding to nucleotide 554, thymine or cytosine at a position corresponding to nucleotide 939, adenine or cytosine at a position corresponding to nucleotide 1198, guanine or thymine at a position corresponding to nucleotide 1242, thymine or cytosine at a position corresponding to nucleotide 1291, cytosine or thymine at a position corresponding to nucleotide 1293, thymine or cytosine at a position corresponding to nucleotide 1299, thymine or cytosine at a position corresponding to nucleotide 1507, cytosine or thymine at a position corresponding to nucleotide 1701, adenine or guanine at a position corresponding to nucleotide 1727, guanine or adenine at a position corresponding to nucleotide 1735, cytosine or thymine at a position corresponding to nucleotide 2023, thynine or guanine at a position corresponding to nucleotide 2254 and thymine or cytosine at a position corresponding to nucleotide 2397.

8. A recombinant organism transformed or transfected with the isolated polynucleotide of claim 7, wherein the organism expresses a Interleukin 4 Receptor Alpha(IL4Rα) protein encoded by the polymorphic variant sequence.

9. The recombinant organism of claim 8 which is a nonhuman transgenic animal.

10. An isolated polypeptide comprising an amino acid sequence which is a polymorphic variant of a reference sequence for the IL4Rα protein or a fragment thereof, wherein the reference sequence comprises SEQ ID NO: 3 and the polymorphic variant is encoded by an isogene defined by one of the haplotypes shown in Table 5.

11. An isolated antibody specific for and immunoreactive with the isolated polypeptide of claim 10.

12. A method for screening for drugs targeting the isolated polypeptide of claim 10 which comprises contacting the IL4Rα polymorphic variant with a candidate agent and assaying for binding activity.

13. A composition comprising at least one genotyping oligonucleotide for detecting a polymorphism in the Interleukin 4 Receptor Alpha(IL4Rα) gene at a polymorphic site selected from PS1, PS2, PS3, PS4, PS6, PS7, PS8, PS9, PS10, PS11, PS12, PS13, PS14, PS15, PS16, PS17, PS18,PS19, PS20, PS21, PS22, PS23, PS25, PS27, PS28, PS30, PS32, PS33, PS34, PS35, PS36, PS37, PS38, PS39, PS40, PS41, PS44, and PS45.

14. The composition of claim 13, wherein the genotyping oligonucleotide is an allele-specific oligonucleotide that specifically hybridizes to an allele of the IL4Rα gene at a region containing the polymorphic site.

15. The composition of claim 14, wherein the allele-specific oligonucleotide comprises a nucleotide sequence selected from the group consisting of of SEQ ID NOS:4-79, the complements of SEQ ID NOS: 4-79, and SEQ ID NOS:80-231.

16. The composition of claim 13, wherein the genotyping oligonucleotide is a primer-extension oligonucleotide.

17. A method for genotyping the Interleukin 4 Receptor Alpha(IL4Rα) gene of an individual, comprising determining for the two copies of the IL4Rα gene present in the individual the identity of the nucleotide pair at each of PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45.

18. The method of claim 17, wherein the determining step comprises:
(a) isolating from the individual a nucleic acid mixture comprising both copies of the IL4Rα gene, or a fragment thereof, that are present in the individual;
(b) amplifying from the nucleic acid mixture a target region containing at least one of the polymorphic sites;
(c) hybridizing a primer extension oligonucleotide to one allele of the amplified target region; (d) performing a nucleic acid template-dependent, primer extension reaction on the hybridized genotyping oligonucleotide in the presence of at least two different terminators of the reaction, wherein said terminators are complementary to the alternative nucleotides present at the polymorphic site; and
(e) detecting the presence and identity of the terminator in the extended genotyping oligonucleotide.

19. A method for haplotyping the Interleukin 4 Receptor Alpha(IL4Rα) gene of an individual which comprises determining, for one copy of the IL4Rα gene present in the individual, the identity of the nucleotide at each of PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45.

20. The method of claim 19, wherein the determining step comprises
(a) isolating from the individual a nucleic acid molecule containing only one of the two copies of the IL4Rα gene, or a fragment thereof, that is present in the individual;
(b) amplifying from the nucleic acid molecule a target region containing at least one of the polymorphic sites;
(c) hybridizing a primer extension oligonucleotide to one allele of the amplified target region;
(d) performing a nucleic acid template-dependent, primer extension reaction on the hybridized genotyping oligonucleotide in the presence of at least two different terminators of the reaction, wherein said terminators are complementary to the alternative nucleotides present at the polymorphic site; and
(e) detecting the presence and identity of the terminator in the extended genotyping oligonucleotide.

21. A method for predicting a haplotype pair for the Interleukin 4 Receptor Alpha(IL4Rα) gene of an individual comprising:
(a) identifying an IL4Rα genotype for the individual at each of PS1, PS2, PS3, PS4, PS6, PS7, PS8, PS9, PS10, PS11, PS12, PS13, PS14, PS15, PS16, PS17, PS18, PS19, PS20, PS21, PS22, PS23, PS25, PS27, PS28, PS30, PS32, PS33, PS34, PS35, PS36, PS37, PS38, PS39, PS40, PS41, PS44, and PS45;
(b) enumerating all possible haplotype pairs which are consistent with the genotype;
(c) accessing data containing the IL4Rα haplotype pairs determined in a reference population; and
(d) assigning a haplotype pair to the individual that is consistent with the data.

22. A method for identifying an association between a trait and at least one haplotype of the Interleukin 4 Receptor Alpha gene which comprises comparing the frequency of the haplotype in a population exhibiting the trait with the frequency of the haplotype in a reference population, wherein the haplotype is selected from haplotype numbers 1-53 shown in Table 5, wherein a higher frequency of the haplotype in the trait population than in the reference population indicates the trait is associated with the haplotype.

24. The method of claim 22, wherein the trait is a clinical response to a drug targeting IL4Rα.

25. A computer system for storing and analyzing polymorphism data for the Interleukin 4 Receptor Alphagene, comprising:
(a) a central processing unit (CPU);
(b) a communication interface;
(c) a display device;
(d) an input device; and
(e) a database containing the polymorphism data;
wherein the polymorphism data comprises the genotypes and haplotype pairs shown in Table 4 and the haplotypes shown in Table 5.

26. A genome anthology for the Interleukin 4 Receptor Alpha(IL4Rα) gene which comprises IL4Rα isogenes defined by haplotypes 1-53 shown in Table 5.

27. A method for haplotyping the Interleukin 4 Receptor Alpha(IL4Rα) gene of an individual which comprises determining whether the individual has one or more haplotypes in Table 5.
